SEQUENCE LISTING (142 sequences)

SEQ ID NO: 1 - DNA, 26 bp, Artificial Sequence
  Synthetic Construct
acacatgctgtgtgccaggcatgccc

SEQ ID NO: 2 - DNA, 28 bp, Artificial Sequence
  Synthetic Construct
agcatgttctagaaagaaagtcatgtac

SEQ ID NO: 3 - DNA, 26 bp, Artificial Sequence
  Synthetic Construct
aggcatgagccacagcacccatgccc

SEQ ID NO: 4 - DNA, 21 bp, Artificial Sequence
  Synthetic Construct
gggcatgtcaaaatcatgcac

SEQ ID NO: 5 - DNA, 23 bp, Artificial Sequence
  Synthetic Construct
ggacatgcgcctgtgccatgccc

SEQ ID NO: 6 - DNA, 23 bp, Artificial Sequence
  Synthetic Construct
cggcatgacgtgttttcatgact

SEQ ID NO: 7 - DNA, 23 bp, Artificial Sequence
  Synthetic Construct
cttcatgtctgacctgcatgtct

SEQ ID NO: 8 - DNA, 21 bp, Artificial Sequence
  Synthetic Construct
gaacatgatcgatccatgtca

SEQ ID NO: 9 - DNA, 24 bp, Artificial Sequence
  Synthetic Construct
gggcatggccttaagcacatgggc

SEQ ID NO: 10 - RNA, 19 nt, Artificial Sequence
  Synthetic Construct
guaacugaaaguuacuuaa

SEQ ID NO: 11 - RNA, 19 nt, Artificial Sequence
  Synthetic Construct
ucaauaaucuccauguuag

SEQ ID NO: 12 - RNA, 19 nt, Artificial Sequence
  Synthetic Construct
cucacaagaaggcuucaau

SEQ ID NO: 13 - RNA, 19 nt, Artificial Sequence
  Synthetic Construct
cacuguuacucagccugaa

SEQ ID NO: 14 - RNA, 19 nt, Artificial Sequence
  Synthetic Construct
gaaggaauuucaggguaaa

SEQ ID NO: 15 - RNA, 19 nt, Artificial Sequence
  Synthetic Construct
ccuggaaccuuccuaacca

SEQ ID NO: 16 - RNA, 19 nt, Artificial Sequence
  Synthetic Construct
gaaacaacuuccacuucca

SEQ ID NO: 17 - RNA, 19 nt, Artificial Sequence
  Synthetic Construct
ggaauugauagccuauaaa

SEQ ID NO: 18 - RNA, 19 nt, Artificial Sequence
  Synthetic Construct
gagcauuuccaauggauua

SEQ ID NO: 19 - RNA, 19 nt, Artificial Sequence
  Synthetic Construct
gaaguacccucaggugacu

SEQ ID NO: 20 - RNA, 19 nt, Artificial Sequence
  Synthetic Construct
caucuagaaagcaucauga

SEQ ID NO: 21 - RNA, 19 nt, Artificial Sequence
  Synthetic Construct
gacuugagggucaacuuua

SEQ ID NO: 22 - RNA, 19 nt, Artificial Sequence
  Synthetic Construct
cacacucugggucccaaca

SEQ ID NO: 23 - RNA, 19 nt, Artificial Sequence
  Synthetic Construct
ccucuacccuccugugaca

SEQ ID NO: 24 - RNA, 19 nt, Artificial Sequence
  Synthetic Construct
cugugucugcccucaaaca

SEQ ID NO: 25 - RNA, 19 nt, Artificial Sequence
  Synthetic Construct
cauaucaauuuaaacuuca

SEQ ID NO: 26 - RNA, 19 nt, Artificial Sequence
  Synthetic Construct
ggacuacaaaucuuaauua

SEQ ID NO: 27 - RNA, 19 nt, Artificial Sequence
  Synthetic Construct
guuguaaucuccauuucag

SEQ ID NO: 28 - RNA, 19 nt, Artificial Sequence
  Synthetic Construct
gaauuccggugaugcgagu

SEQ ID NO: 29 - RNA, 19 nt, Artificial Sequence
  Synthetic Construct
cuaacgauuugguggugaa

SEQ ID NO: 30 - RNA, 19 nt, Artificial Sequence
  Synthetic Construct
guaaagcccugaacuauca

SEQ ID NO: 31 - RNA, 19 nt, Artificial Sequence
  Synthetic Construct
cauccauaaagaaggcaua

SEQ ID NO: 32 - RNA, 19 nt, Artificial Sequence
  Synthetic Construct
ccacuuauuggcccucauu

SEQ ID NO: 33 - RNA, 19 nt, Artificial Sequence
  Synthetic Construct
gacuuguucugucgcuucu

SEQ ID NO: 34 - RNA, 19 nt, Artificial Sequence
  Synthetic Construct
aguuuagucuacgguccuc

SEQ ID NO: 35 - RNA, 19 nt, Artificial Sequence
  Synthetic Construct
uggaugauaaaauaacuag

SEQ ID NO: 36 - RNA, 19 nt, Artificial Sequence
  Synthetic Construct
cuuuuucugcucuccuccc

SEQ ID NO: 37 - DNA, 20 bp, Artificial Sequence
  Synthetic Construct
tgcactgccttgtccatgtt

SEQ ID NO: 38 - DNA, 20 bp, Artificial Sequence
  Synthetic Construct
gtagtgacacaggcgaggac

SEQ ID NO: 39 - DNA, 20 bp, Artificial Sequence
  Synthetic Construct
cacgtgccccataaaagctg

SEQ ID NO: 40 - DNA, 23 bp, Artificial Sequence
  Synthetic Construct
tgcaagggttcaaagaggaaact

SEQ ID NO: 41 - DNA, 22 bp, Artificial Sequence
  Synthetic Construct
atgagttgccaaatgcatagcc

SEQ ID NO: 42 - DNA, 23 bp, Artificial Sequence
  Synthetic Construct
tgaaaagcaaaccacatttcgag

SEQ ID NO: 43 - DNA, 22 bp, Artificial Sequence
  Synthetic Construct
acagatctaggaagcaaagtgc

SEQ ID NO: 44 - DNA, 20 bp, Artificial Sequence
  Synthetic Construct
tgtcgcccaggctggtctcc

SEQ ID NO: 45 - DNA, 20 bp, Artificial Sequence
  Synthetic Construct
tcttaagttggtgaaatttg

SEQ ID NO: 46 - DNA, 20 bp, Artificial Sequence
  Synthetic Construct
gggctggccatgtagtagtc

SEQ ID NO: 47 - DNA, 19 bp, Artificial Sequence
  Synthetic Construct
ctcctcgaggtgcctctgt

SEQ ID NO: 48 - DNA, 20 bp, Artificial Sequence
  Synthetic Construct
actcagtttggtatccaggg

SEQ ID NO: 49 - DNA, 20 bp, Artificial Sequence
  Synthetic Construct
tgtttgtctcttccaggtgg

SEQ ID NO: 50 - DNA, 20 bp, Artificial Sequence
  Synthetic Construct
tgctatggcccgaagttggg

SEQ ID NO: 51 - DNA, 18 bp, Artificial Sequence
  Synthetic Construct
caggaacagctcccgacc

SEQ ID NO: 52 - DNA, 19 bp, Artificial Sequence
  Synthetic Construct
ctgtgtgaatccagcaccc

SEQ ID NO: 53 - DNA, 18 bp, Artificial Sequence
  Synthetic Construct
agctgggacggtccattc

SEQ ID NO: 54 - DNA, 19 bp, Artificial Sequence
  Synthetic Construct
gagaattcagacagtgagg

SEQ ID NO: 55 - DNA, 19 bp, Artificial Sequence
  Synthetic Construct
tgcggaagcaagacagtgc

SEQ ID NO: 56 - DNA, 18 bp, Artificial Sequence
  Synthetic Construct
gctggcctccaggatcgc

SEQ ID NO: 57 - DNA, 20 bp, Artificial Sequence
  Synthetic Construct
cctgtaaacagatggcaagc

SEQ ID NO: 58 - DNA, 21 bp, Artificial Sequence
  Synthetic Construct
aggtcttatgattgtcttccc

SEQ ID NO: 59 - DNA, 21 bp, Artificial Sequence
  Synthetic Construct
cctttcagcctatgaacatgc

SEQ ID NO: 60 - DNA, 19 bp, Artificial Sequence
  Synthetic Construct
gcactctagcctgggtgac

SEQ ID NO: 61 - DNA, 21 bp, Artificial Sequence
  Synthetic Construct
tcttcatggcacctggattcc

SEQ ID NO: 62 - DNA, 20 bp, Artificial Sequence
  Synthetic Construct
acagcgtcactaatctctcc

SEQ ID NO: 63 - DNA, 21 bp, Artificial Sequence
  Synthetic Construct
cctaaaatggagagttccagg

SEQ ID NO: 64 - DNA, 20 bp, Artificial Sequence
  Synthetic Construct
ctgcctgatctccatgtagc

SEQ ID NO: 65 - DNA, 22 bp, Artificial Sequence
  Synthetic Construct
cactgtagttgagaaaatgagc

SEQ ID NO: 66 - DNA, 22 bp, Artificial Sequence
  Synthetic Construct
tgaagagaatgagaaaatgtgg

SEQ ID NO: 67 - DNA, 21 bp, Artificial Sequence
  Synthetic Construct
caaagtgctaggattacaggc

SEQ ID NO: 68 - DNA, 20 bp, Artificial Sequence
  Synthetic Construct
ctgaaagtggctcagggttg

SEQ ID NO: 69 - DNA, 19 bp, Artificial Sequence
  Synthetic Construct
gagcggatcctgaaacggt

SEQ ID NO: 70 - DNA, 21 bp, Artificial Sequence
  Synthetic Construct
tccattctatctgccccacct

SEQ ID NO: 71 - DNA, 21 bp, Artificial Sequence
  Synthetic Construct
ttgtttttgagggggaggagg

SEQ ID NO: 72 - DNA, 21 bp, Artificial Sequence
  Synthetic Construct
acagaagcttgcagggaacct

SEQ ID NO: 73 - DNA, 26 bp, Artificial Sequence
  Synthetic Construct
actattactgctaactaacttatgga

SEQ ID NO: 74 - protein, 11 aa, Artificial Sequence
  Synthetic Construct
GSSLKKKKRLS

SEQ ID NO: 75 - protein, 14 aa, Artificial Sequence
  Synthetic Construct
LEDGKVLEEALQVL

SEQ ID NO: 76 - DNA, 22 bp, Artificial Sequence
  Synthetic Construct
cccatttagagaattcgggaag

SEQ ID NO: 77 - DNA, 22 bp, Artificial Sequence
  Synthetic Construct
agttaaggcagagttcacaaag

SEQ ID NO: 78 - DNA, 22 bp, Artificial Sequence
  Synthetic Construct
tctgccttaactcaaagattcc

SEQ ID NO: 79 - DNA, 20 bp, Artificial Sequence
  Synthetic Construct
catcctcctccttcatttgc

SEQ ID NO: 80 - DNA, 20 bp, Artificial Sequence
  Synthetic Construct
gcaaatgaaggaggaggatg

SEQ ID NO: 81 - DNA, 22 bp, Artificial Sequence
  Synthetic Construct
tcagatttatgctgtagatgcc

SEQ ID NO: 82 - DNA, 22 bp, Artificial Sequence
  Synthetic Construct
acagcataaatctgaagaggac

SEQ ID NO: 83 - DNA, 21 bp, Artificial Sequence
  Synthetic Construct
cctggaggatatgacactgac

SEQ ID NO: 84 - DNA, 21 bp, Artificial Sequence
  Synthetic Construct
tgtcagtgtcatatcctccag

SEQ ID NO: 85 - DNA, 20 bp, Artificial Sequence
  Synthetic Construct
gtcagaaaccacaagaagcc

SEQ ID NO: 86 - DNA, 22 bp, Artificial Sequence
  Synthetic Construct
taaccagatccaggcagcctac

SEQ ID NO: 87 - DNA, 20 bp, Artificial Sequence
  Synthetic Construct
tgtccaatggcacagaggag

SEQ ID NO: 88 - DNA, 20 bp, Artificial Sequence
  Synthetic Construct
ccattggacattgccatcac

SEQ ID NO: 89 - DNA, 18 bp, Artificial Sequence
  Synthetic Construct
tgttgcaggaaatggtgc

SEQ ID NO: 90 - DNA, 19 bp, Artificial Sequence
  Synthetic Construct
caccatttcctgcaacacc

SEQ ID NO: 91 - DNA, 21 bp, Artificial Sequence
  Synthetic Construct
gctgaaacagtccatttaccc

SEQ ID NO: 92 - DNA, 22 bp, Artificial Sequence
  Synthetic Construct
ctgctgctgaaaaggctgtgac

SEQ ID NO: 93 - DNA, 22 bp, Artificial Sequence
  Synthetic Construct
actgctgaataggcacagaggg

SEQ ID NO: 94 - DNA, 22 bp, Artificial Sequence
  Synthetic Construct
gtgcctattcagcagtttctac

SEQ ID NO: 95 - DNA, 20 bp, Artificial Sequence
  Synthetic Construct
tgcaagaacagcttaagacc

SEQ ID NO: 96 - DNA, 22 bp, Artificial Sequence
  Synthetic Construct
tggtcttaagctgttcttgcac

SEQ ID NO: 97 - DNA, 25 bp, Artificial Sequence
  Synthetic Construct
actgatgtttattttctgtcaacct

SEQ ID NO: 98 - DNA, 17 bp, Artificial Sequence
  Synthetic Construct
gcttgcgggaggggggc

SEQ ID NO: 99 - DNA, 20 bp, Artificial Sequence
  Synthetic Construct
ctgctggatgtgaaatggcg

SEQ ID NO: 100 - DNA, 19 bp, Artificial Sequence
  Synthetic Construct
accgccatttcacatccag

SEQ ID NO: 101 - DNA, 21 bp, Artificial Sequence
  Synthetic Construct
tgggcaatatagtgagacctc

SEQ ID NO: 102 - DNA, 19 bp, Artificial Sequence
  Synthetic Construct
tattgcccaagctggtctc

SEQ ID NO: 103 - DNA, 19 bp, Artificial Sequence
  Synthetic Construct
ataatttcgcagggcatgg

SEQ ID NO: 104 - DNA, 21 bp, Artificial Sequence
  Synthetic Construct
agataacaggtatgagccacc

SEQ ID NO: 105 - DNA, 19 bp, Artificial Sequence
  Synthetic Construct
gcagtgagttgagattggg

SEQ ID NO: 106 - DNA, 19 bp, Artificial Sequence
  Synthetic Construct
cccaatctcaactcactgc

SEQ ID NO: 107 - DNA, 21 bp, Artificial Sequence
  Synthetic Construct
gggcaatatagtgaaaccctg

SEQ ID NO: 108 - DNA, 21 bp, Artificial Sequence
  Synthetic Construct
ggtttcactatattgcccagg

SEQ ID NO: 109 - DNA, 22 bp, Artificial Sequence
  Synthetic Construct
aaggaaagaaaagggatgacac

SEQ ID NO: 110 - DNA, 22 bp, Artificial Sequence
  Synthetic Construct
tcatcccttttctttccttcac

SEQ ID NO: 111 - DNA, 21 bp, Artificial Sequence
  Synthetic Construct
aatgcctccagatattttgcc

SEQ ID NO: 112 - DNA, 20 bp, Artificial Sequence
  Synthetic Construct
ttggcaaaatatctggaggc

SEQ ID NO: 113 - DNA, 21 bp, Artificial Sequence
  Synthetic Construct
ttcaacacctgcactattgac

SEQ ID NO: 114 - DNA, 22 bp, Artificial Sequence
  Synthetic Construct
caggtccaaagtgtcaatagtg

SEQ ID NO: 115 - DNA, 21 bp, Artificial Sequence
  Synthetic Construct
aaatgcctctgaaagggaatg

SEQ ID NO: 116 - DNA, 22 bp, Artificial Sequence
  Synthetic Construct
agtctcttgcctttatgaaacc

SEQ ID NO: 117 - DNA, 22 bp, Artificial Sequence
  Synthetic Construct
gcagaaacctgcatataaagcc

SEQ ID NO: 118 - DNA, 20 bp, Artificial Sequence
  Synthetic Construct
tggagtcaattgcatcaacc

SEQ ID NO: 119 - DNA, 20 bp, Artificial Sequence
  Synthetic Construct
tatgtgcctaccttgtaccc

SEQ ID NO: 120 - DNA, 20 bp, Artificial Sequence
  Synthetic Construct
tgggtacaaggtaggcacat

SEQ ID NO: 121 - DNA, 24 bp, Artificial Sequence
  Synthetic Construct
ggaaattcatctctcaccacatga

SEQ ID NO: 122 - DNA, 24 bp, Artificial Sequence
  Synthetic Construct
ggtgagagatgaatttcctgaaca

SEQ ID NO: 123 - DNA, 24 bp, Artificial Sequence
  Synthetic Construct
tgctaacttatggaagtggaagtt

SEQ ID NO: 124 - DNA, 24 bp, Artificial Sequence
  Synthetic Construct
acttccacttccataagttagcag

SEQ ID NO: 125 - DNA, 21 bp, Artificial Sequence
  Synthetic Construct
aatgagggtcctggaaagctc

SEQ ID NO: 126 - DNA, 22 bp, Artificial Sequence
  Synthetic Construct
tgagctttccaggaccctcatt

SEQ ID NO: 127 - DNA, 23 bp, Artificial Sequence
  Synthetic Construct
tctatgtgtgtttggtctcctcc

SEQ ID NO: 128 - DNA, 24 bp, Artificial Sequence
  Synthetic Construct
gaggagaccaaacacacatagagt

SEQ ID NO: 129 - DNA, 21 bp, Artificial Sequence
  Synthetic Construct
tttggggccaggggaaactac

SEQ ID NO: 130 - DNA, 21 bp, Artificial Sequence
  Synthetic Construct
cctggccccaaagtttattgc

SEQ ID NO: 131 - DNA, 25 bp, Artificial Sequence
  Synthetic Construct
agctttttagtgggagtttttctgg

SEQ ID NO: 132 - DNA, 21 bp, Artificial Sequence
  Synthetic Construct
acattccagaaaaactcccac

SEQ ID NO: 133 - DNA, 21 bp, Artificial Sequence
  Synthetic Construct
gggctcaagcaataatctcac

SEQ ID NO: 134 - DNA, 20 bp, Artificial Sequence
  Synthetic Construct
gagcccagaagtttgagacc

SEQ ID NO: 135 - DNA, 21 bp, Artificial Sequence
  Synthetic Construct
gggactacaggcatacatcac

SEQ ID NO: 136 - DNA, 21 bp, Artificial Sequence
  Synthetic Construct
tggtgatgtatgcctgtagtc

SEQ ID NO: 137 - DNA, 21 bp, Artificial Sequence
  Synthetic Construct
tgtgtgttttttgagacaggg

SEQ ID NO: 138 - DNA, 19 bp, Artificial Sequence
  Synthetic Construct
atgatcaccactgcactcc

SEQ ID NO: 139 - DNA, 20 bp, Artificial Sequence
  Synthetic Construct
cttttctggcctctggaatg

SEQ ID NO: 140 - DNA, 21 bp, Artificial Sequence
  Synthetic Construct
tcattccagaggccagaaaag

SEQ ID NO: 141 - DNA, 25 bp, Artificial Sequence
  Synthetic Construct
ttctttattgtcctccatgttttcc

SEQ ID NO: 142 - DNA, 20 bp, Artificial Sequence
  Synthetic Construct
cagagtgcactttcacatgc

(19) United States
(12) Patent Application Publication
(10) Pub. No.: US 2023/0057430 A1
(43) Pub. Date: Feb. 23, 2023

(54) INHIBITION OF TAP63 REGULATED ONCOGENIC LONG NON-CODING RNAS (TROLLS) IN THE TREATMENT OF CANCER

(71) Applicant: H. LEE MOFFITT CANCER CENTER AND RESEARCH INSTITUTE, INC., Tampa, FL (US)

(72) Inventors: Elsa R. FLORES, Tampa, FL (US); Marco NAPOLI, Tampa, FL (US)

(21) Appl. No.: 17/792,661

(22) PCT Filed: Jan. 13, 2020
(86) PCT No.: PCT/US2021/013321

§ 371 Date: Jul. 13, 2022

Related U.S. Application Data
(60) Provisional application No. 62/960,623, filed on Jan. 13, 2020.
(60) Provisional application No. 62/990,784, filed on Mar. 17, 2020.
(60) Provisional application No. 63/118,044, filed on Nov. 25, 2020.
(60) Provisional application No. 63/124,046, filed on Dec. 10, 2020.

(51) Int. Cl. C12N 15/113

(52) U.S. Cl. CPC C12N 15/1135; C12N 2310/14; C12N 2320/30

(57) ABSTRACT

Disclosed herein are 2 novel long non-coding RNAs (lncRNAs), TROLL-2 and TROLL-3. It is shown herein that lncRNAs TROLL-2 and TROLL-3, as well as their effector WDR26, are suitable targets for cancer therapies and can be used to make prognostic determinations about a cancer and determine if immune checkpoint inhibitors should be used to treat a cancer.

This application claims the benefit of U.S. Provisional Application No. 63/124,046, filed on Dec. 10, 2020; U.S. Provisional Application No. 63/118,044, filed on Nov. 25, 2020, U.S. Provisional Application No. 62/990,784, filed on Mar. 17, 2020, and U.S. Provisional Application No. 62/960,623, filed on Jan. 13, 2020. Applications which are incorporated herein by reference in their entirety.

### I. BACKGROUND

Cancer metastasis is the leading cause of death in cancer patients. Multiple pathways have been found to increase cancer progression and metastasis including the activation of the PI3K/AKT pathway and the gain-of-function mutation of the tumor suppressor TP53, which are the two most frequent driving mutations in a broad variety of human cancers. Therefore, investigating the mechanistic interplay between these pathways is of the utmost importance for the identification of novel therapeutic opportunities against the progression of metastatic cancers.

### II. SUMMARY

Disclosed are methods and compositions related to combination therapies that target the p53 family members and methods of treating cancer utilizing said combination therapies.

A method of treating, inhibiting, reducing, decreasing, ameliorating, and/or preventing a cancer and/or metastasis (such as, for example a Tap63 regulated cancer and/or a cancer comprising a p53 mutation including but not limited to breast cancer (but not limited to triple negative breast cancer), lung cancer (including, but not limited to adenocarcinomas and squamous cell carcinomas), ovarian (including, but not limited to serous and non-serous adenocarcinomas), liver cancer, colon cancer, or melanoma) in a subject comprising knocking down expression of one or any combination of two or more of TROLL-1, TROLL-2, TROLL-3, TROLL-4, TROLL-5, TROLL-6, TROLL-7, TROLL-8, and/or TROLL-9 in the subject. In some aspect, the method does NOT involve knocking down the expression of TROLL-2. In some aspects treatment is administered to an established or metastatic cancer.

The method of treating, inhibiting, reducing, decreasing, ameliorating, and/or preventing a cancer and/or metastasis in a subject of any preceding aspect, wherein expression of one or any combination of two or more of TROLL-1, TROLL-2, TROLL-3, TROLL-4, TROLL-5, TROLL-6, TROLL-7, TROLL-8, and/or TROLL-9 is knocked down through the administration of one or more RNA-targeted therapeutics.

The method of treating, inhibiting, reducing, decreasing, ameliorating, and/or preventing a cancer and/or metastasis in a subject of any preceding aspect, wherein the one or more RNA-targeted therapeutics comprises antisense oligonucleotides, siRNA, shRNA, ribozymes, transcription activator-like effector nucleases (TALEN), zinc finger nucleases (ZFNs) and/or clustered regularly interspaced short palindromic repeats/associated (CRISPR/Cas) nucleases.

In one aspect disclosed herein are methods of treating, inhibiting, reducing, decreasing, ameliorating, and/or preventing a cancer and/or metastasis in a subject of any preceding aspect, wherein the expression of TROLL-1 is knocked down by targeting the ENST00000441085.5 and/or ENST00000452465.1 isoform of TROLL-1 (for example by administering an siRNA as set forth in SEQ ID NO: 10, SEQ ID NO 11, and/or SEQ ID NO: 12); wherein the expression of TROLL-2 is knocked down by targeting the RPSAP52/NR_026825.2 isoform of TROLL-2 (for example by administering an siRNA as set forth in SEQ ID NO: 13, SEQ ID NO 14, and/or SEQ ID NO: 15); wherein the expression of TROLL-3 is knocked down by targeting the TRAF3IP2-AS1/NR_034108.1, TRAF3IP2-AS1/NR_034109.1, TRAF3IP2-AS1/NR_034110.1, and/or TRAF3IP2-AS1/NR_034111.1 isoform of TROLL-3 (for example by administering an siRNA as set forth in SEQ ID NO: 16, SEQ ID NO 17, and/or SEQ ID NO: 18); wherein TROLL-4 expression is knocked down by targeting the NR_015410 isoform of TROLL-4 (for example by administering an siRNA as set forth in SEQ ID NO: 19, SEQ ID NO 20, and/or SEQ ID NO: 21); wherein the expression of TROLL-5 is knocked down by targeting the LINC00514/NR_033861.1 isoform of TROLL-5 (for example by administering an siRNA as set forth in SEQ ID NO: 22, SEQ ID NO 23, and/or SEQ ID NO: 24); wherein TROLL-6 expression is knocked down by targeting the RP11-138I1.3 isoform of TROLL-6 (for example by administering an siRNA as set forth in SEQ ID NO: 25, SEQ ID NO 26, and/or SEQ ID NO: 27); wherein the expression of TROLL-7 is knocked down by targeting the MALAT1/NR_002819.4, MALAT1/NR_144567.1, and/or MALAT1/NR_144568.1 isoform of TROLL-7 (for example by administering an siRNA as set forth in SEQ ID NO: 28, SEQ ID NO 29, and/or SEQ ID NO: 30); wherein the expression of TROLL-8 is knocked down by targeting the ENST00000554568.1 isoform of TROLL-8 (for example by administering an siRNA as set forth in SEQ ID NO: 31, SEQ ID NO 32, and/or SEQ ID NO: 33); and/or wherein TROLL-9 expression is knocked down by targeting the RP11-126K1.6 isoform of TROLL-9 ((for example by administering an siRNA as set forth in SEQ ID NO: 34, SEQ ID NO 35, and/or SEQ ID NO: 36).

In one aspect, disclosed herein is an siRNA comprising SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ D NO: 33, SEQ ID NO: 34, SEQ ID NO: 35, or SEQ ID NO: 36.

Also disclosed herein are pharmaceutical compositions comprising a therapeutically effective amount of one or more of SEQ ID NO: 10, SEQ ID NO: 11, SEQ ID NO: 12, SEQ D NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, or SEQ ID NO: 36. For example, a composition comprising any combination at least 2, 3, 4, 5, 6, 7, 8, 9, 10, 11, 12, 13, 4, 15, 16, 17, 18, 19, 20, 21, 22, 23, 24, 25, 26, or all 27 siRNAs of any preceding aspect.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A-1H show the identification of TAp63-regulated lncRNAs in human breast cancer progression. FIG. 1A shows a Heatmap visualization of the 9 conserved lncRNAs differentially expressed in WT and TAp63−/− mammary epithelial cells (MECs). 1b Heatmap visualization of the 9 conserved lncRNAs differentially expressed in the MCF10A human breast cancer progression model. 1c qRT-PCR of the 9 conserved lncRNAs in WT and TAp63−/− MECs. Data are mean±SD, n=3, * vs. WT, P<0.005, two-tailed Student's t test. 1d qRT-PCR of the 9 conserved lncRNAs in CA1D cells infected with either non targeting (NT) or TAp63 targeting gRNA. 3 days and 6 days indicate the time of Cas9 induction via doxycycline. Data are mean±SD, n=3, * vs. NT gRNA, P<0.005, two-tailed Student's t test. 1e-1h Quantification for cell migration (1e), cell invasion (1f), EdU incorporation (1g), and annexin V positivity (1h) of CA1D cells transfected with siRNAs against the indicated lncRNAs. Data are mean±SD, n=3, * vs. siControl, P<0.005, two-tailed Student's t test.

FIG. 2A-2I show the identification of TAp63-regulated lncRNAs in human breast cancer progression. 2a qRT-PCR of the 9 conserved lncRNAs differentially expressed in the MCF10A human breast cancer progression model. Data are mean±SD, n=3, * vs. MCF10A cells, P<0.005, two-tailed Student's t test. 2b Frequency of insertions/deletions occurring in the TAp63 locus in CA1D cells infected with TAp63 targeting gRNA. The number of days indicate the time of Cas9 induction via doxycycline. 2c qRT-PCR for TAp63 in CA1D cells treated as in (2b). Data are mean±SD, n=3, * vs. non targeting no doxy, P<0.005, two-tailed Student's t test. 2d Representative western blot of Myc-tag 981 TAp63γ in WT Mammary Epithelial Cells (MECs) transfected with either pcDNA3 Myc-982 TAp63γ or pcDNA3, as a negative control. 2e qRT-PCR of the indicated lncRNAs in the 983 same cells treated as in (2d). 2f Representative western blot of Myc-tag TAp63γ in CA1D 984 cells transfected with either pcDNA3 Myc-TAp63γ or pcDNA3, as a negative control. 2g qRT-PCR of the indicated lncRNAs in the same cells treated as in (2f). 2h Table listing the TAp63 binding sites on the promoters of the indicated lncRNAs. Number of mismatches to the TAp63 consensus binding site are shown in red text. MM indicates the number of mismatches to the TAp63 consensus binding site for TROLL-1 (SEQ ID NO: 1), TROLL-2 (SEQ ID NO: 2), TROLL-3 (SEQ ID NO: 3), TROLL-4 (SEQ ID NO: 4), TROLL-5 (SEQ ID NO: 5), TROLL-6 (SEQ ID NO: 6), TROLL-7 (SEQ ID NO: 7), TROLL-8 (SEQ ID NO: 8), and TROLL-9 (SEQ ID NO: 9). Spacer indicates the number of nucleotides between two half sites. 2i qRT-PCR of TAp63 ChIP assays on the promoters of the indicated lncRNAs. CA1D cells infected with TAp63 targeting gRNA were either left untreated (No doxy) or treated with doxycycline (+ doxy). Data are mean±SD, n=3, * vs. No doxy non-specific binding site (NS BS), P<0.005, two-tailed Student's t test.

FIG. 3A-3N show that TROLL-2 and TROLL-3 promote the tumorigenic and metastatic potential of human breast cancers. 3a and 3b Quantification of the in situ hybridization (ISH) scores of TROLL-2 (3a) and TROLL-3 (3b) in a tissue microarray (TMA) of breast cancer progression (BR480a, Table 4).

TABLE 4

| | TMA ID and | |
|---|---|---|
| TMA Name | Source | Pathology Diagnosis |
| Breast cancer | BR480a | Normal breast tissue (n = 2) |
| progression TMA - | (US Biomax) | Lobular hyperplasia (n = 10) |
| Biomax | | Ductal carcinoma in situ |
| | | (n = 11) |
| | | Invasive breast cancer |
| | | (n = 22) |
| Invasive breast | BR20837a | Grade 1 (n = 4) |
| cancer TMA - | (US Biomax) | Grade 2 (n = 34) |
| Biomax | | Grade 3 (n = 39) |
| Invasive breast | Breast TMA (26), | Grade 1 (n = 18) |
| cancer TMA - | Tayside Tissue | Grade 2 (n = 58) |
| Dundee | Bank | Grade 3 (n = 79) |
| Invasive breast | TMA-5, Moffitt | Triple negative breast cancer |
| cancer TMA - | Cancer Center | (TNBC) (n = 43) |
| Moffitt | | Non-TNBC (n = 25) |
| Colon cancer | CO961 | Normal colon tissue (n = 1) |
| TMA | (US Biomax) | Polyp (n = 5) |
| | | Adenoma (n = 6) |
| | | Adenocarcinoma (n = 61) |
| Lung | BC04002a | Normal lung tissue (n = 2) |
| adenocarcinoma | (US Biomax) | Benign lesion (n = 9) |
| TMA | | Adenocarcinoma (LUAD) |
| | | (n = 16) |
| Lung squamous | BC04002a | Normal lung tissue (n = 2) |
| cell carcinoma | (US Biomax) | Benign lesion (n = 9) |
| TMA | | Squamous cell carcinoma |
| | | (LUSC) (n = 18) |
| | | Metastatic LUSC (n = 10) |
| Melanoma TMA - | ME1004 | Benign nevus (n = 16) |
| Biomax | (US Biomax) | Stage 1 (n = 3) |
| | | Stage 2 (n = 30) |
| | | Stage 3 (n = 12) |
| | | Stage 4 (n = 1) |
| | | Unknown stage (n = 31) |
| Melanoma TMA - | TMA-4, Moffitt | Control case (n = 6) |
| Moffitt | Cancer Center | Stage 3 (n = 39) |
| | | Stage 4 (n = 61) |
| Ovarian cancer - | OV1005b | Normal ovary (n = 6) |
| Biomax | (US Biomax) | Serous adenocarcinoma |
| | | (n = 32) |
| | | Non-Serous adenocarcinoma |
| | | (n = 13) |

Data were analyzed with two-way ANOVA. * vs. normal breast tissue, P<0.005. § vs. lobular hyperplasia, P<0.005. # vs. ductal carcinoma in situ, P<0.005. 3c and 3d Correlation of the ISH scores of TROLL-2 (3c) and TROLL-3 (3d) with TP53 status in the indicated TMA of invasive breast cancers. * vs. WT p53, P<0.005, Welchs Student's t test. 3e and 3f Kaplan-Meier curves of overall breast cancer survival data showing the prognostic value of TROLL-2 (3e) and TROLL-3 (3f) in tumors of the indicated TMA with higher or lower than median levels of the considered lncRNA. P=0.0480 (3e). P=0.0243 (3f). 3g Representative hematoxylin and eosin (H&E) stained cross sections of mammary adenocarcinomas derived from CA1D cells infected with the indicated doxycycline-inducible shRNAs, and injected in the 4th mammary fat pad pairs of 6-weeks old athymic nu/nu mice. The mice were fed doxycycline for the duration of the experiments to downregulate the lncRNAs of interest. 3h Tumor volume quantification of the tumors described in (3g). n=10 tumors, * vs. shNT, P<0.005, two-tailed Student's t test. 3i Representative H&E stained cross sections of mammary adenocarcinomas derived from MDA MB-231 cells infected with the indicated doxycycline-inducible shRNAs, and injected in the 4th mammary fat pad pairs of 6-weeks old athymic nu/nu mice. The mice were fed doxycycline for the duration of the experiments to downregulate the lncRNAs of interest. 3j Tumor volume quantification of the tumors described in (3i). n=10 tumors, * vs. shNT, P<0.005, two-tailed Student's t test. 3k Representative H&E stained cross sections of lung colonies derived from CA1D cells infected with the indicated doxycycline-inducible shRNAs, and injected in the tail vein of 6-week old athymic nu/nu mice (n=5 mice for all groups). The mice were fed doxycycline for the duration of the experiments to downregulate the lncRNA of interest. Black arrows indicate representative lung colonies. 3l Quantification of the lung colonies described in (3k). n=5 mice for all groups, * vs. NT, P<0.005, two-tailed Student's t test. 3m Representative H&E stained cross sections of lung colonies derived from MDA MB-231 cells infected with the indicated doxycycline-inducible shRNAs, and injected in the tail vein of 6-week old athymic nu/nu mice (n=5 mice for all groups). The mice were fed doxycycline for the duration of the experiments to downregulate the lncRNA of interest. Black arrows indicate representative lung colonies. 3n Quantification of the lung colonies described in (3m). n=5 mice for all groups, * vs. NT, P<0.005, two-tailed Student's t test.

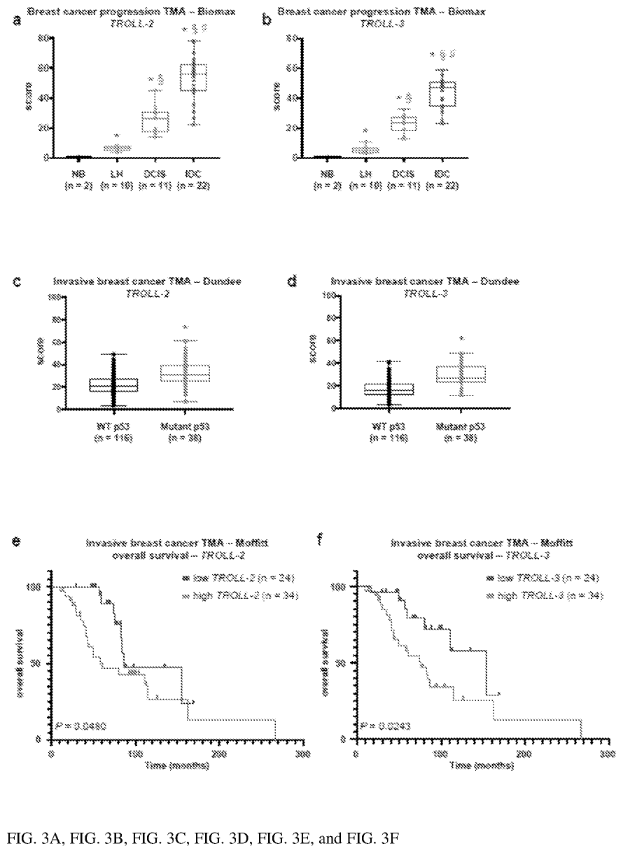

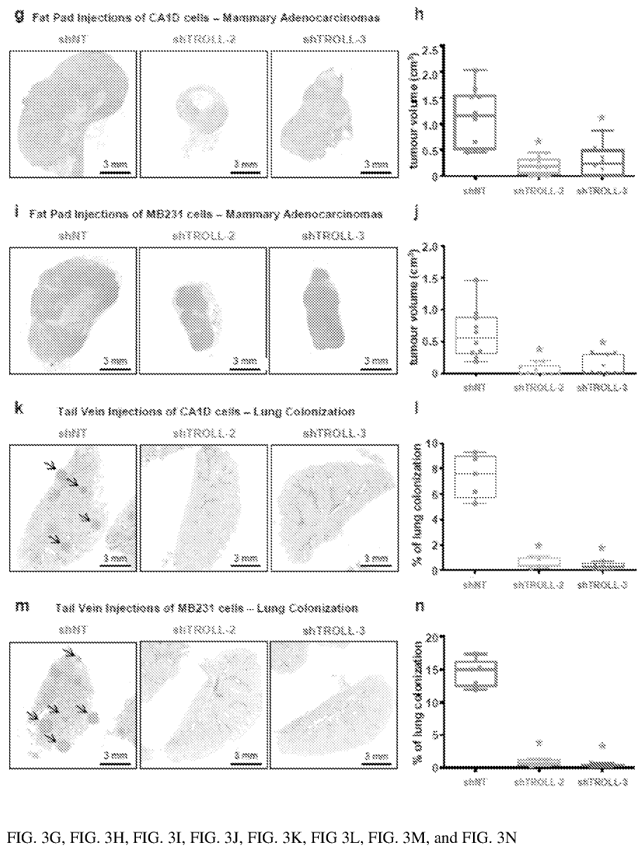

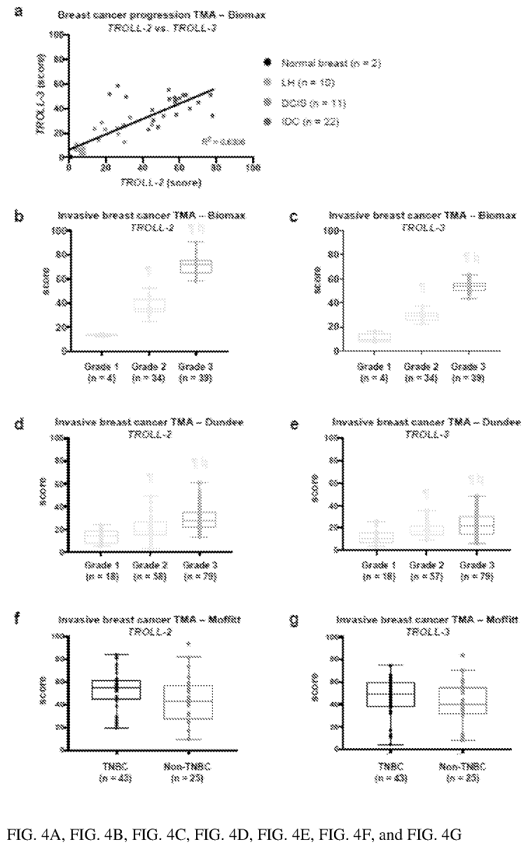

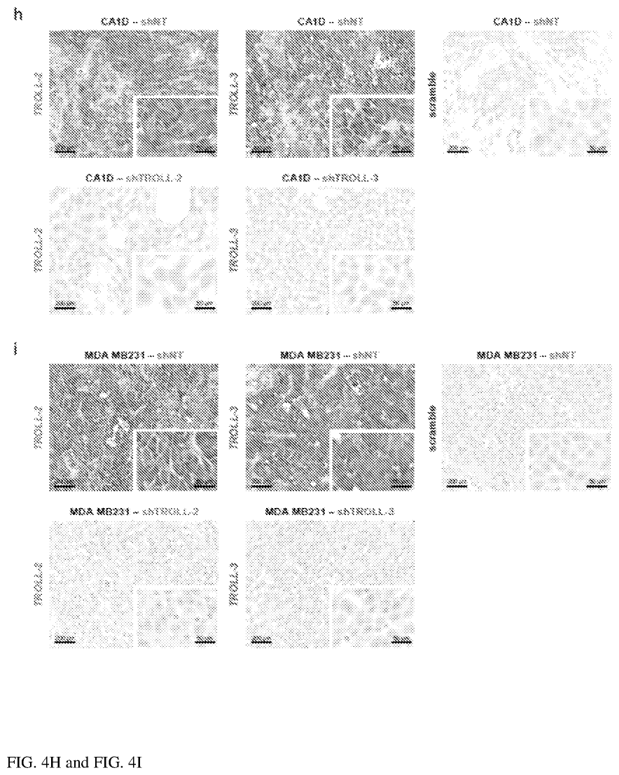

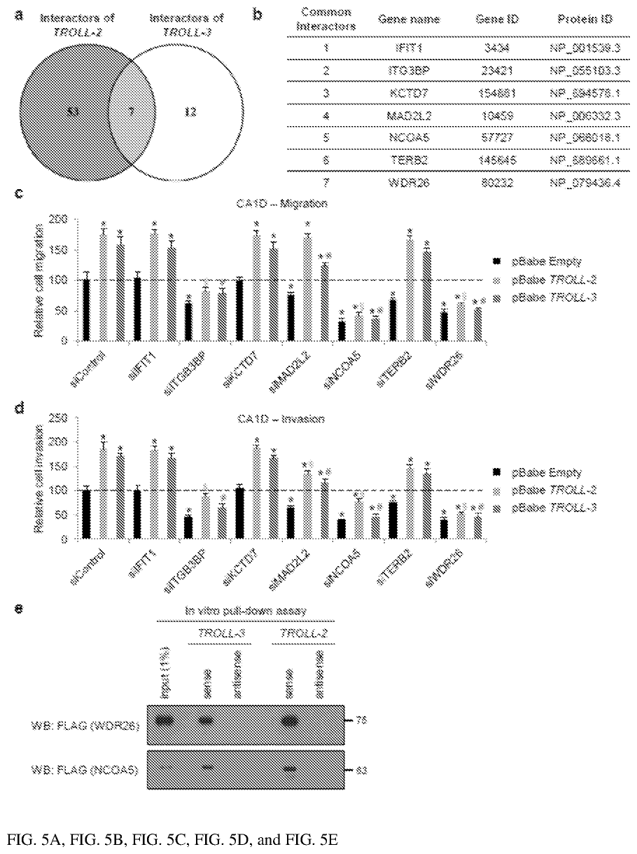

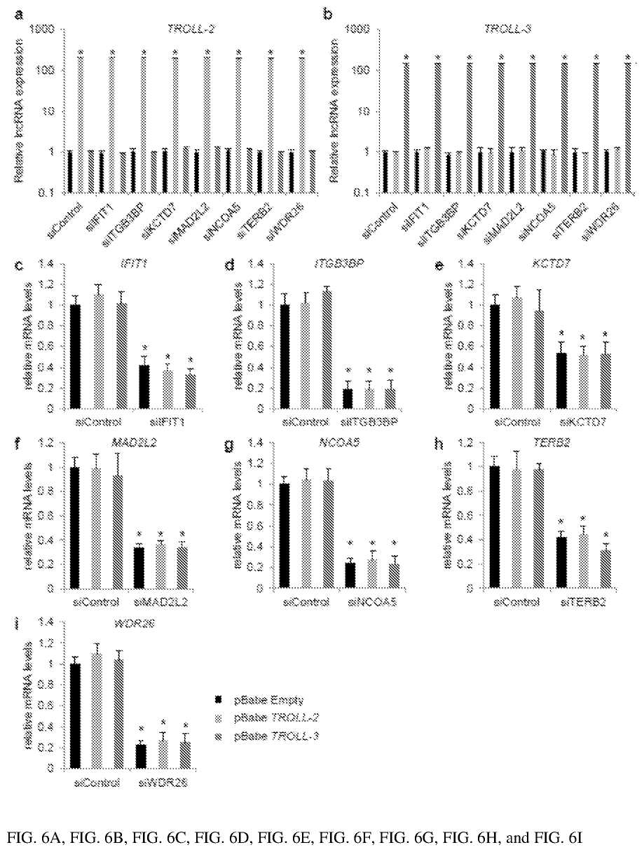

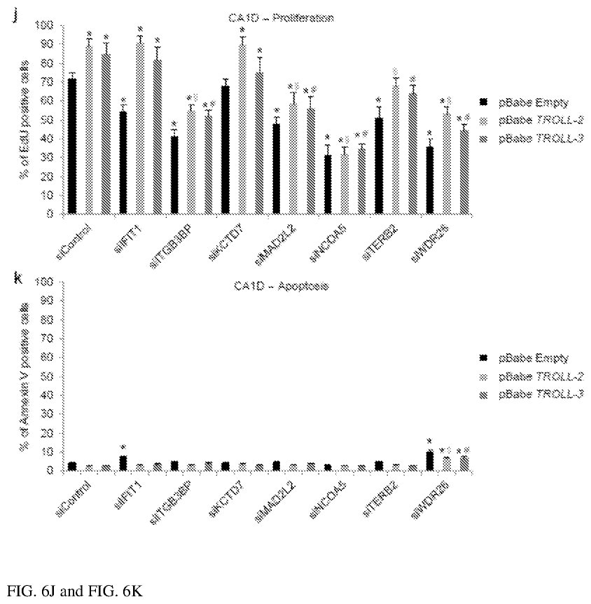

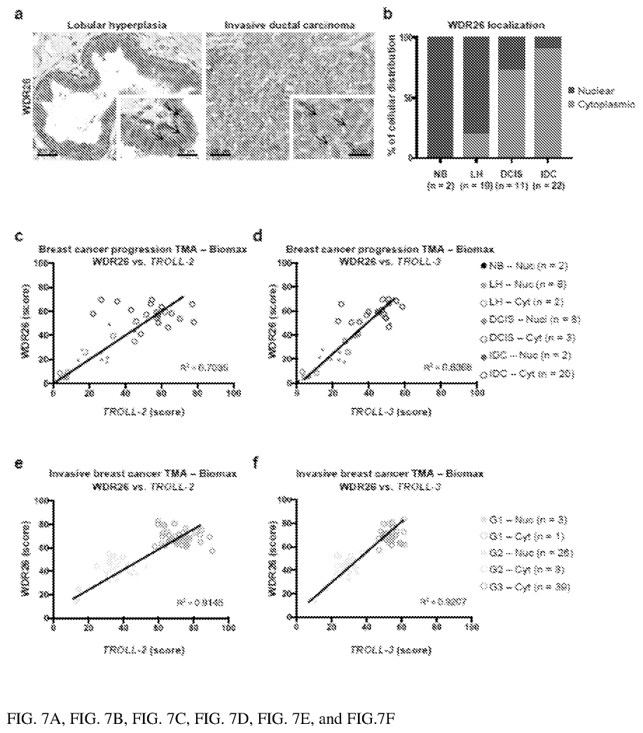

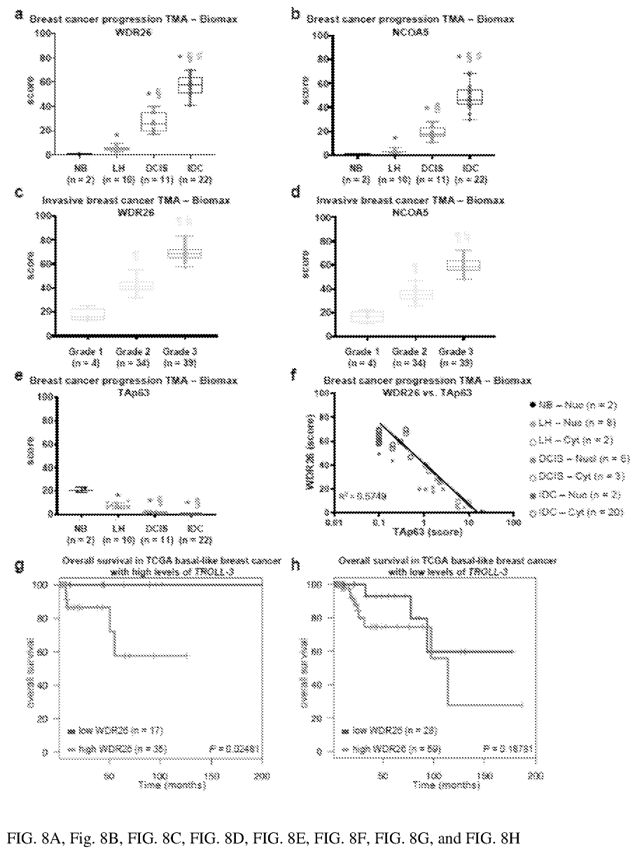

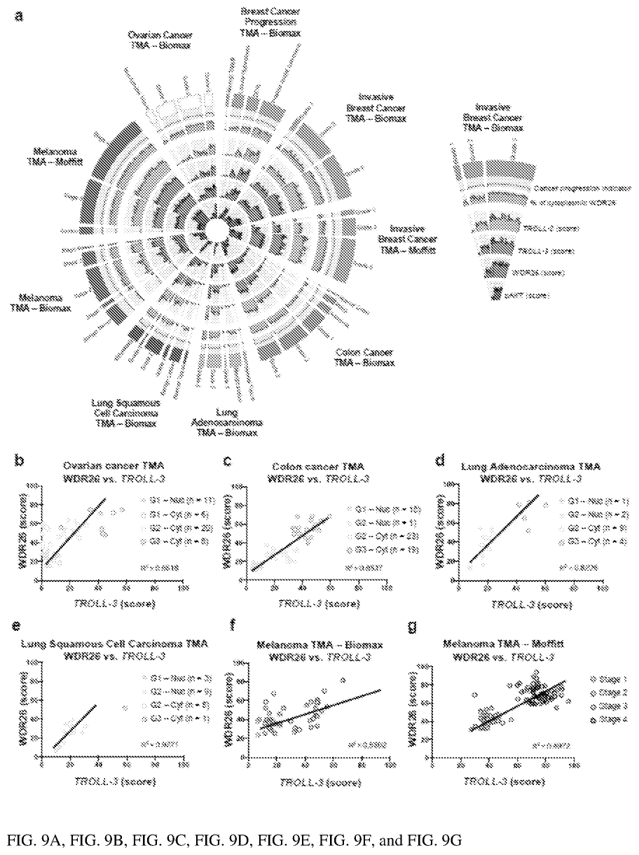

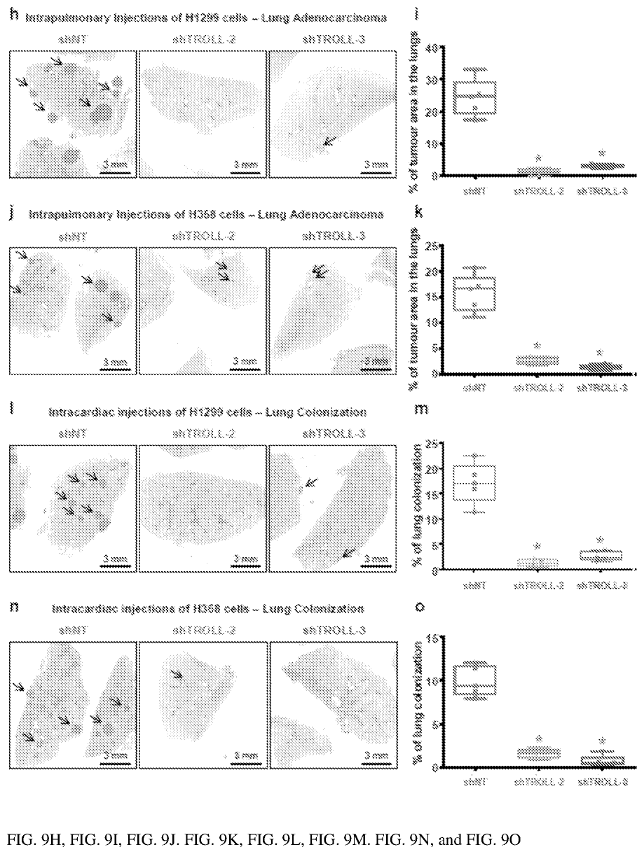

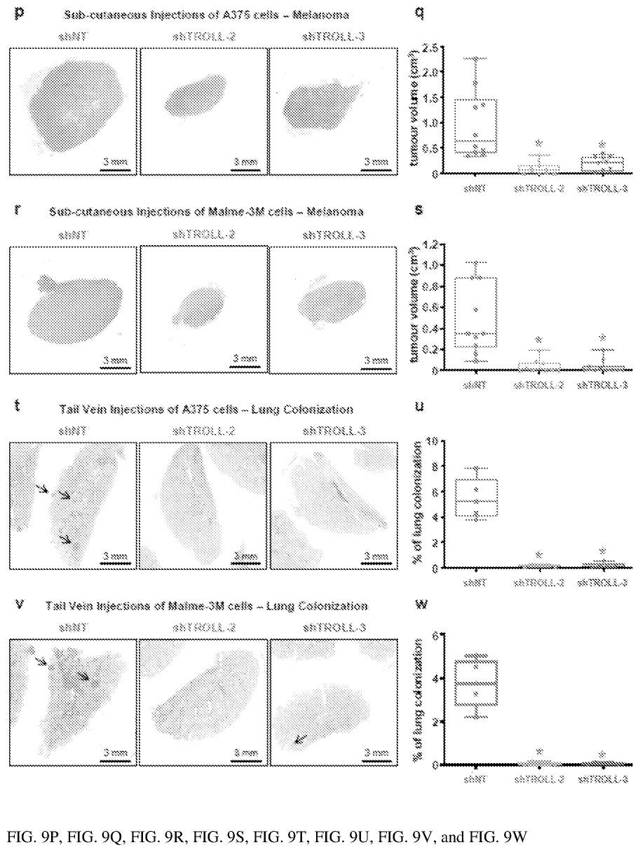

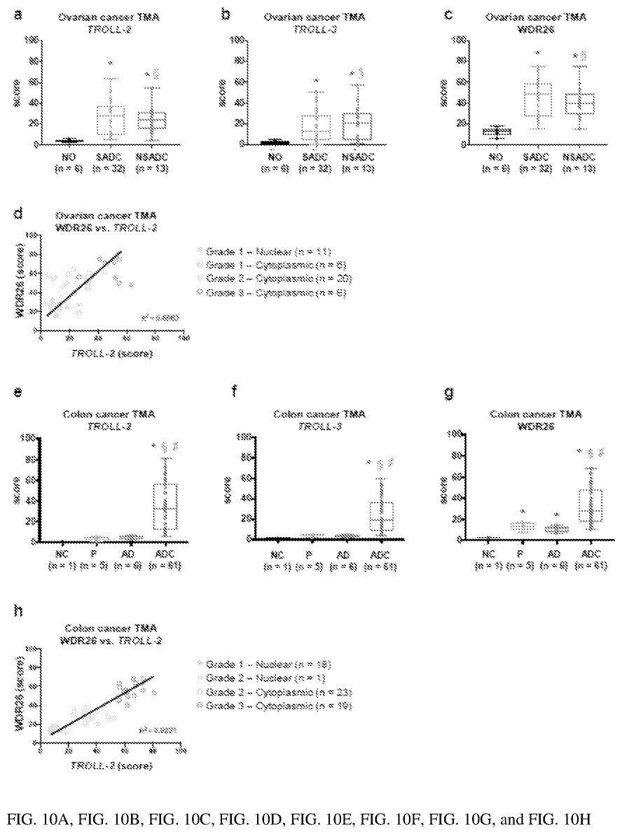

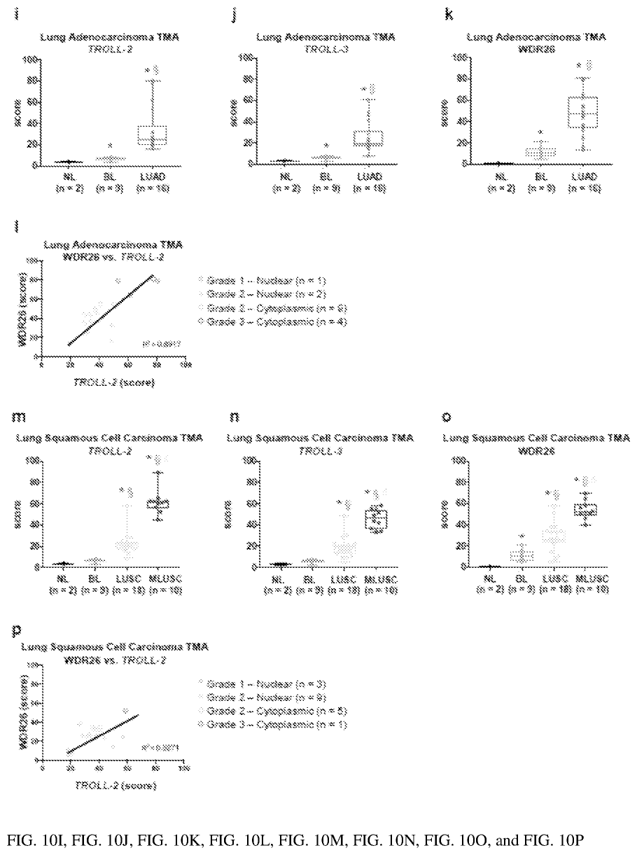

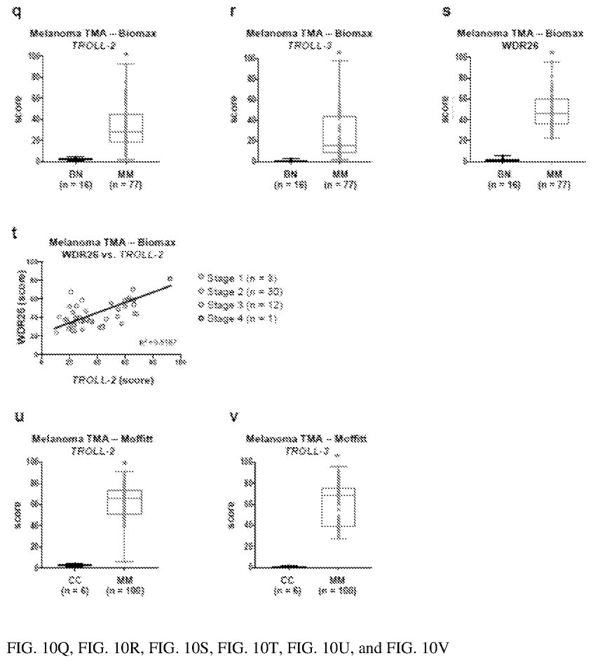

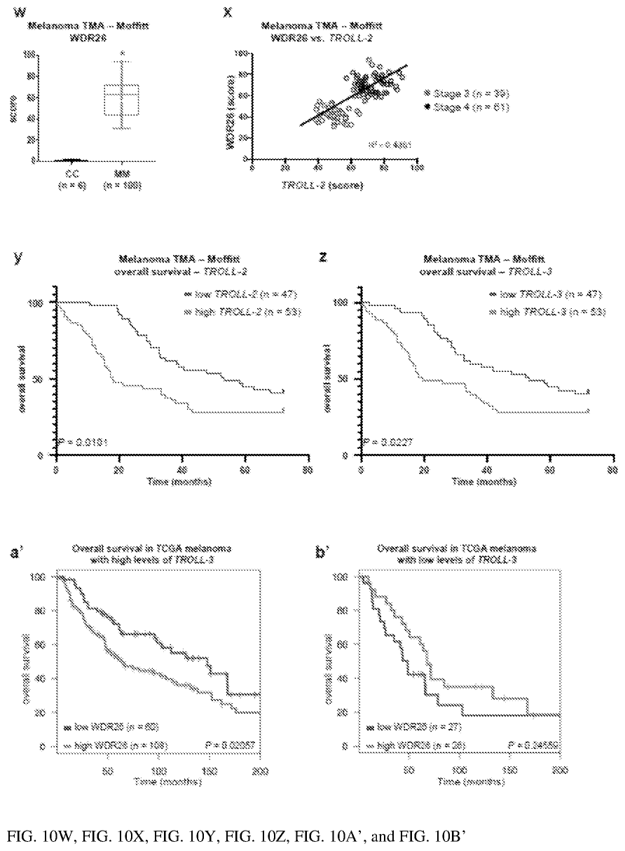

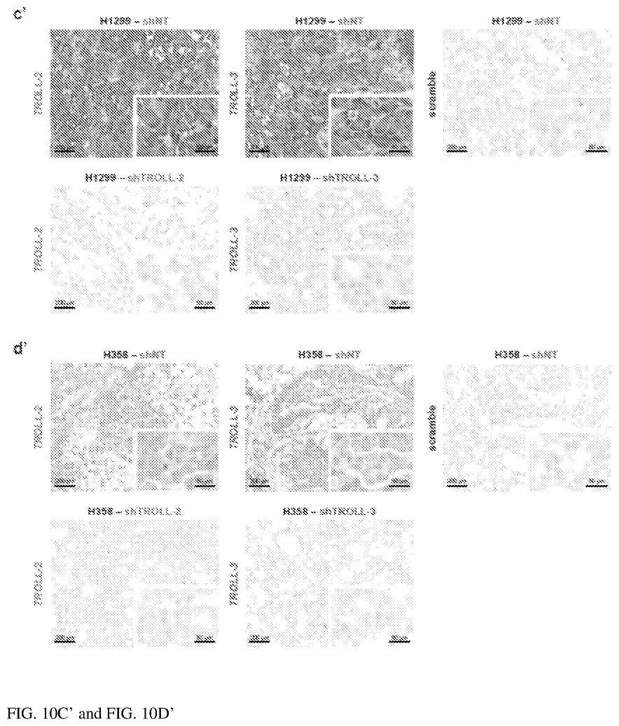

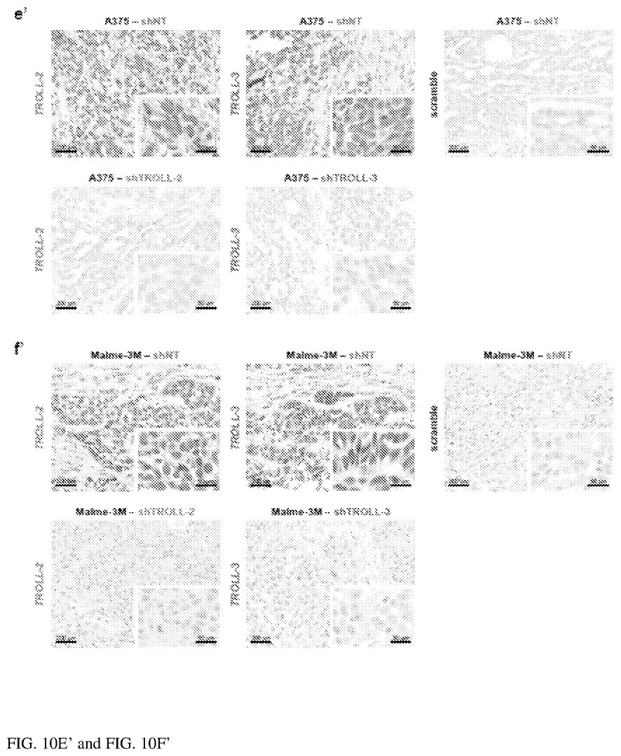

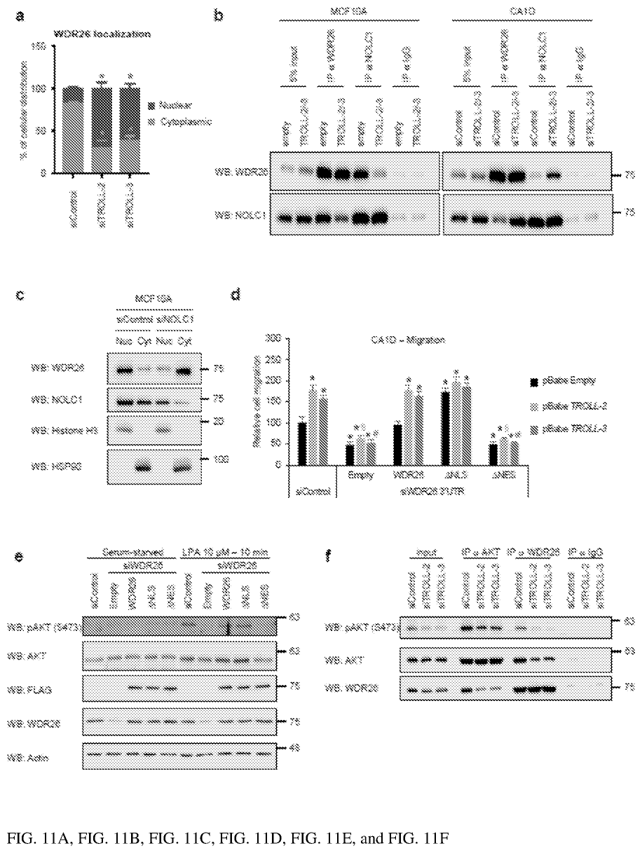

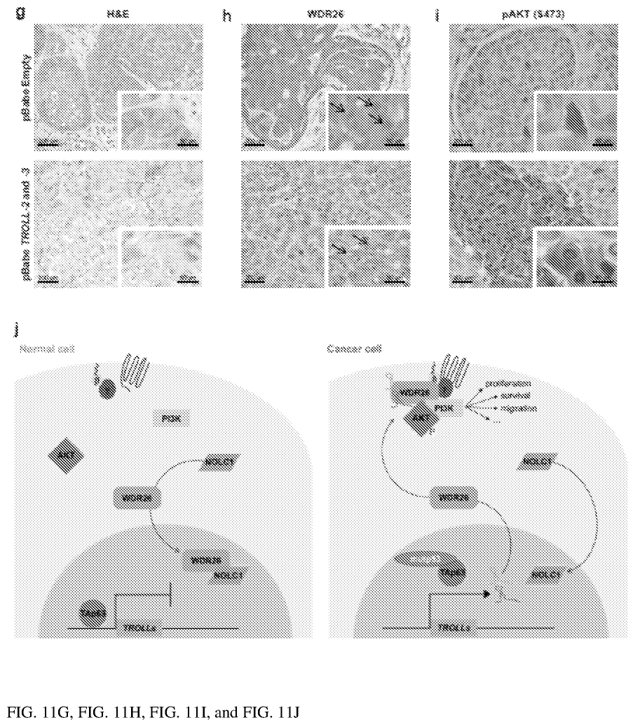

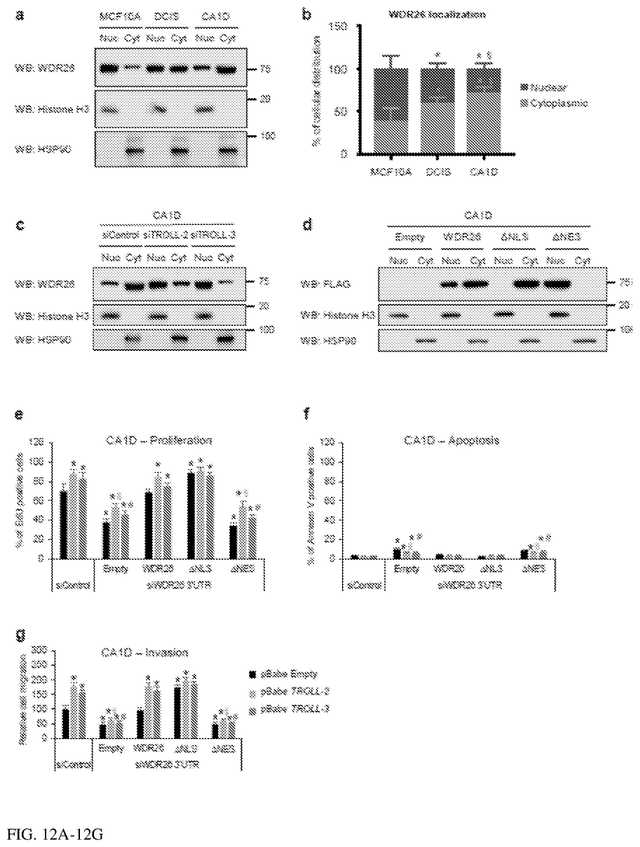

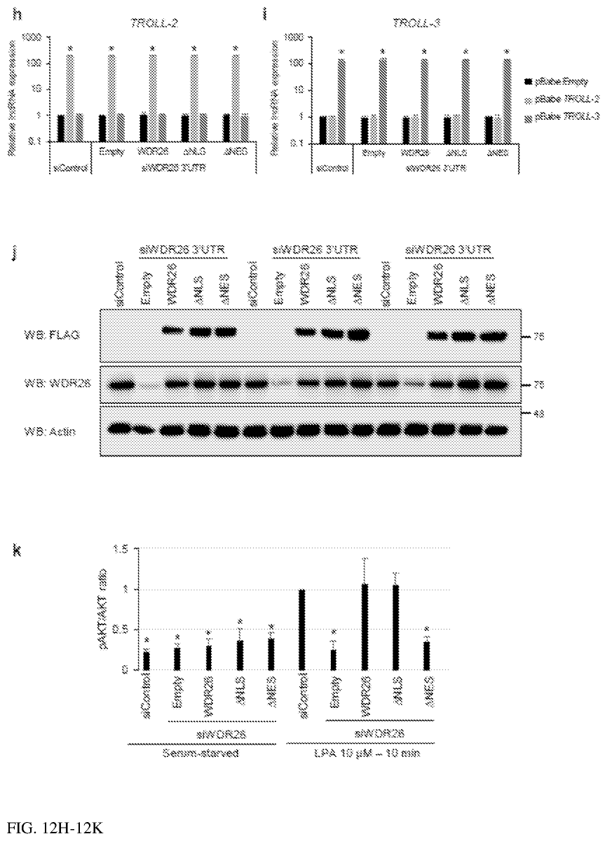

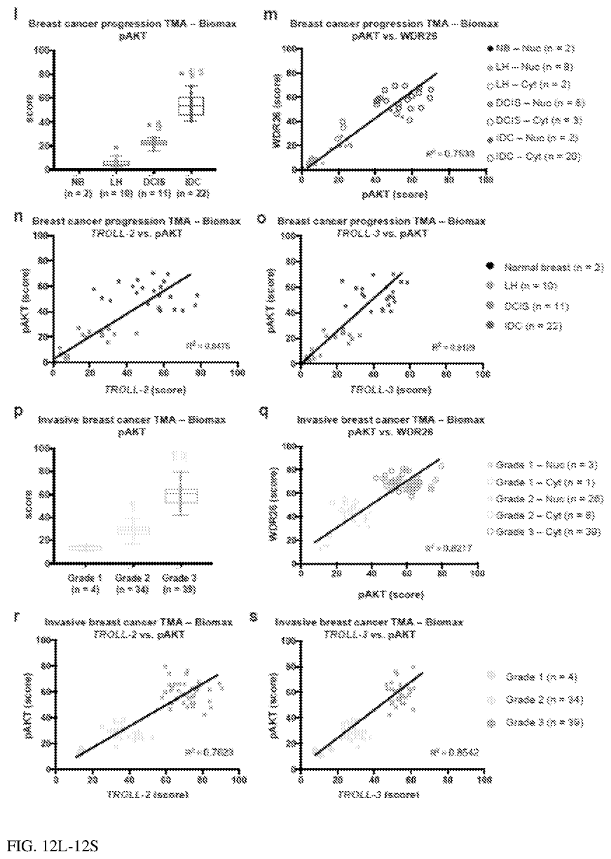

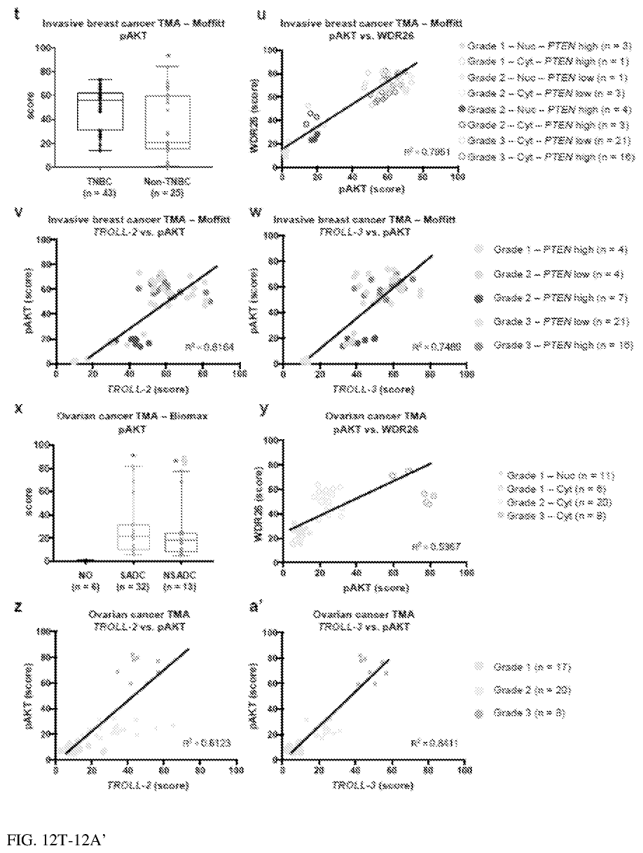

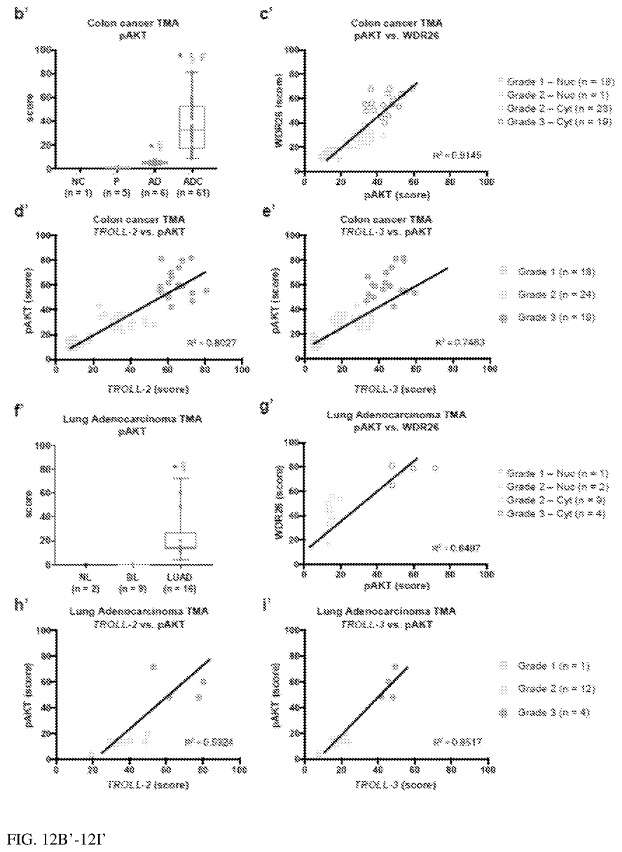

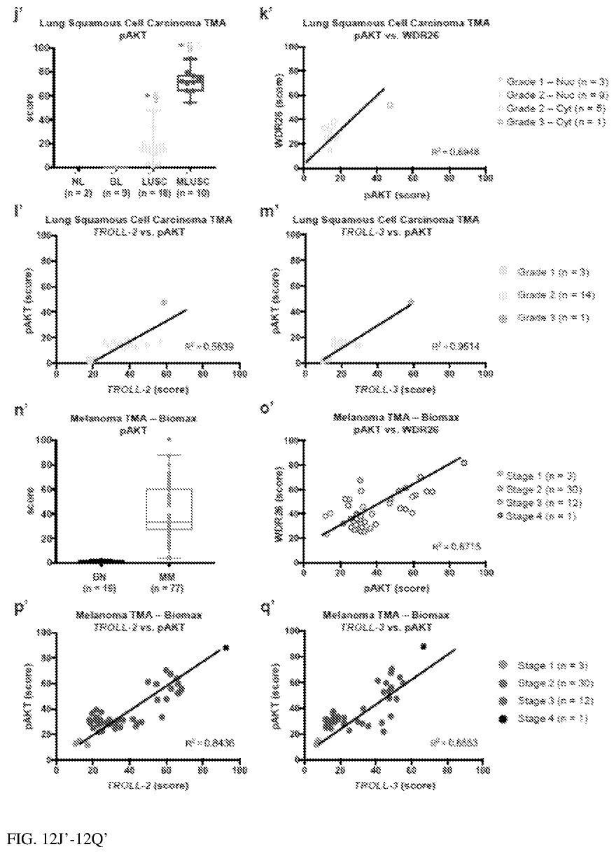

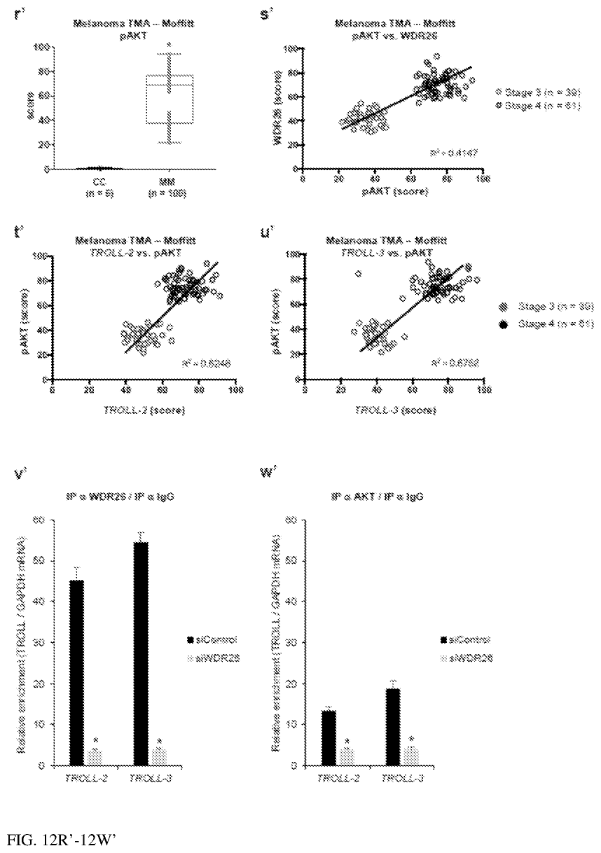

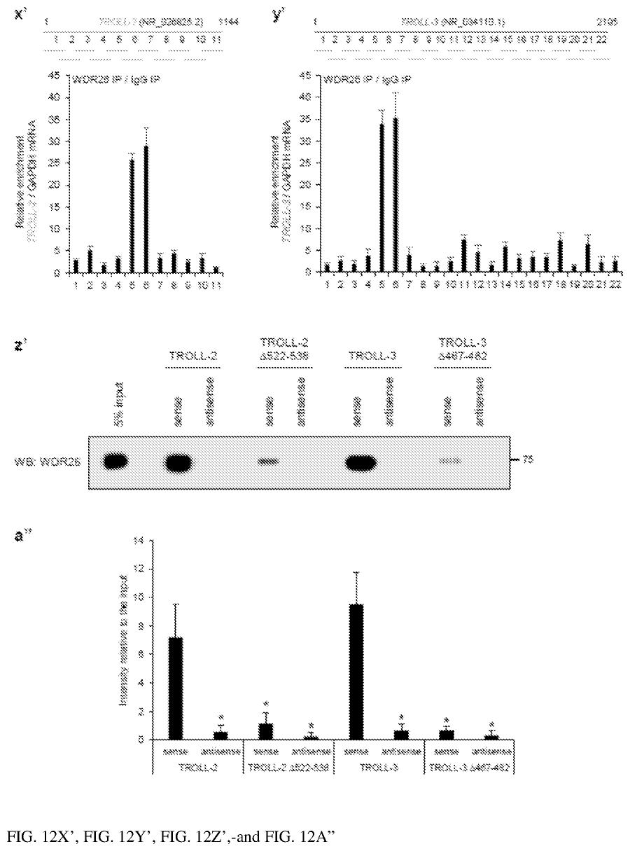

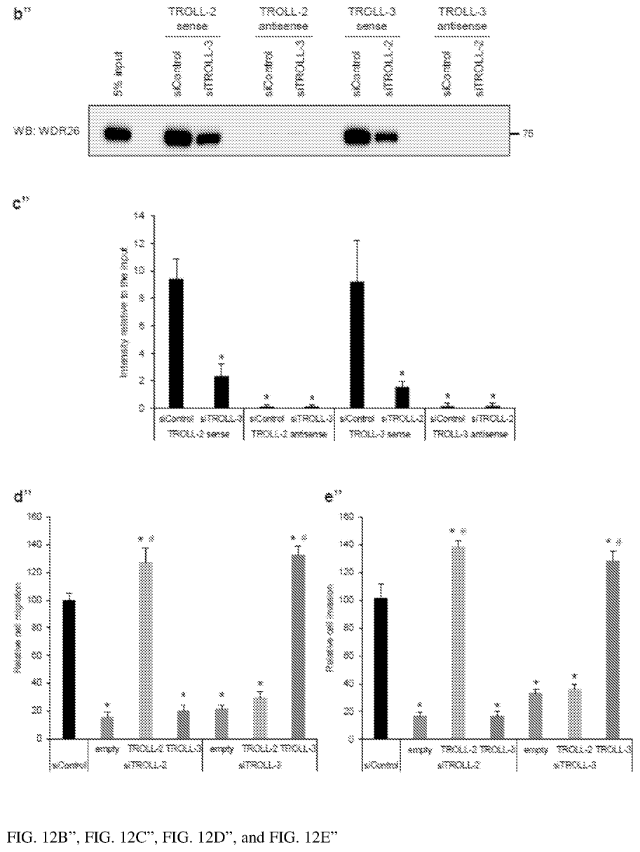

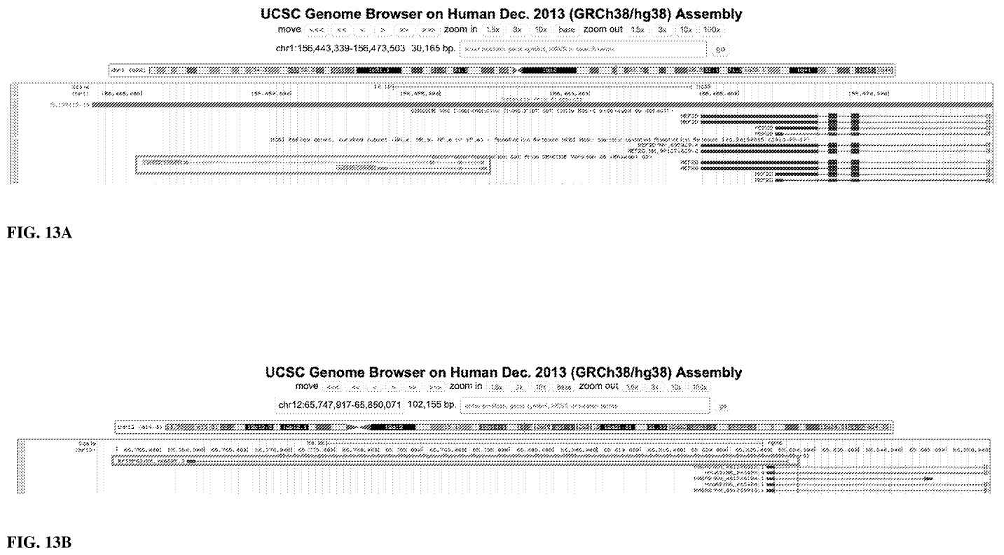

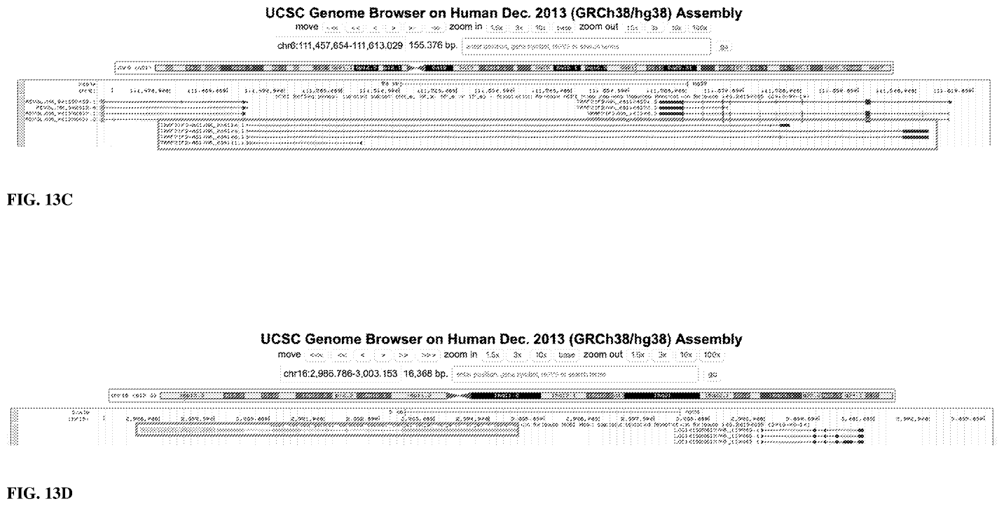

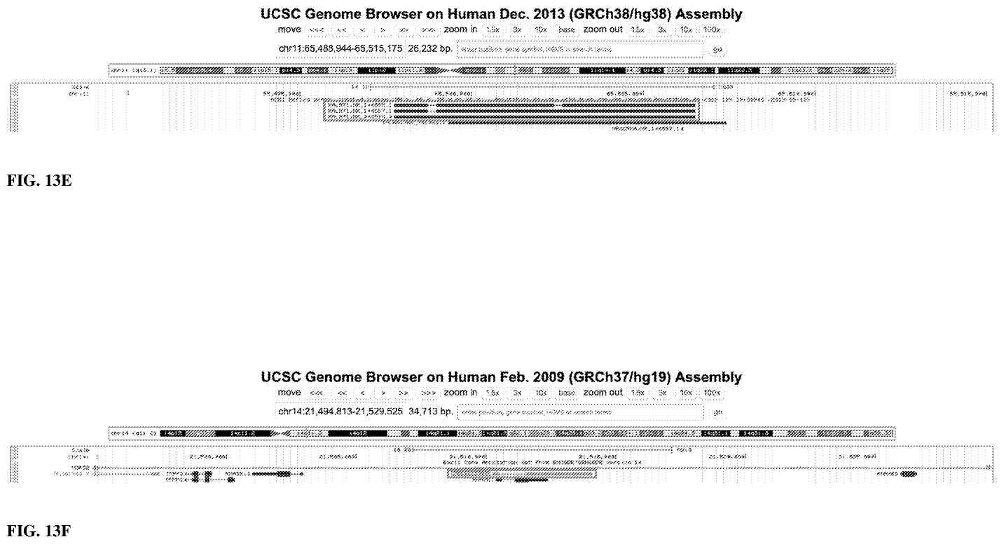

FIGS. 4A-4I show TROLL-2 and TROLL-3 promote the tumorigenic and metastatic potential of human breast cancers. 4a Correlation of the ISH scores of TROLL-2 and TROLL-3 shown in FIG. 3a,b. 4b and 4c Correlation of the ISH scores of TROLL-2 (4b) and TROLL-3 (4c) with tumor grade in a TMA of invasive breast cancers (BR20837a, Table 4). Data were analysed with two-way ANOVA. ¶ vs. grade 1, P<0.005. ♮ vs. grade 2, P<0.005. 4d and 4e Correlation of the ISH scores of TROLL-2 (4d) and TROLL-3 (4e) with tumor grade in the indicated TMA of invasive breast cancers. Data were analysed with two-way ANOVA. ¶ vs. grade 1, P<0.005. ♮ vs. grade 2, P<0.005. 4f and 4g Correlation of the ISH scores of TROLL-2 (4f) and TROLL-3 (4g) with TNBC vs. non-TNBC cases in the indicated TMA of invasive breast cancers. * vs. TNBC, P<0.005, Welchs t test. 4h Representative images of ISH for TROLL-2 (left panels) and TROLL-3 (middle panels) in tumors derived from CA1D cells shown in FIG. 3g,h. The LNA probe was detected and visualized via a chromogenic reaction (purple), while Nuclear Fast Red™ was used as a counterstain. A scramble LNA probe (right panel) was used as a negative control. 4i Representative images of ISH for TROLL-2 (left panels) and TROLL-3 (middle panels) in tumors derived from MDA MB-231 cells shown in FIG. 3k,l. The LNA probe was detected and visualized via a chromogenic reaction (purple), while Nuclear Fast Red™ was used as a counterstain. A scramble LNA probe (right panel) was used as a negative control.

FIGS. 5A-5E show TROLL-2 and TROLL-3 mediate their tumorigenic and metastatic activities through interaction with WDR26. FIG. 5a shows a Venn diagram of the proteins interacting with TROLL-2 and TROLL-3. FIG. 5b shows a table listing the 7 common interactors of TROLL-2 and TROLL-3. FIGS. 5c and 5d show quantification of cell migration (5c) and invasion (5d) of CA1D cells overexpressing either TROLL-2, TROLL-3, or the empty vector, and transfected with the indicated siRNAs. Data are mean±SD and analysed with two-tailed Student's t test, n=3, * vs. pBabe Empty siControl, P<0.005. § vs. pBabe TROLL-2 siControl, P<0.005. # vs. pBabe TROLL-3 siControl, P<0.005. 5e Representative western blot for the indicated proteins pulled down by streptavidin precipitation of the indicated in vitro synthesized and biotinylated lncRNAs.

FIGS. 6A-6K show TROLL-2 and TROLL-3 mediate their tumorigenic and metastatic activities through WDR26. 6a, 6b, 6b, 6c, 6d, 6e, 6f, 6g, 6h, and 6i qRT-PCR for TROLL-2 (6a), TROLL-3 (6b), IFIT1 (6c), ITGB3BP (6d), KCDT7 (6e), MAD2L2 (6f), NCOA5 (6g), TERB2 (6h), and WDR26 (6i) in CA1D cells overexpressing either TROLL-2, TROLL-3, or the empty vector as a negative control, and transfected with the indicated siRNAs. Data are mean±SD and analysed with two-tailed Student's t test, n=3, * vs. pBabe Empty siControl, P<0.005. 6j and 6k Quantification for EdU 6(j) and annexin V (6k) of CA1D cells overexpressing either TROLL-2, TROLL-3, or the empty vector, and transfected with the indicated siRNAs. Data are mean±SD and analysed with two-tailed Student's t test, n=3, * vs. pBabe Empty siControl, P<0.005. § vs. pBabe TROLL-2 siControl, P<0.005. # vs. pBabe TROLL-3 siControl, P<0.005.

FIGS. 7A-7F show WDR26 cytoplasmic localization correlates with breast cancer progression. 7a Representative images of immunohistochemistry (IHC) for WDR26 in lobular hyperplasia (left) and invasive ductal carcinoma (right). Positive signal is brown. Hematoxylin (purple) was used as a counterstain. Black arrows indicate nuclei positive for WDR26. 7b Quantification of the percentage of WDR26 cellular distribution in a tissue microarray of breast cancer progression (BR480a, Table 4). 7c and 7d Correlation of the IHC score and cellular distribution of WDR26 with the ISH score of TROLL-2 (7c) and TROLL-3 (d) in the same tissue microarray as in (7b). The colour legend in (7d) also applies to (7c). 7e and 7f Correlation of the IHC score and cellular distribution of WDR26 with the ISH score of TROLL-2 (7e) and TROLL-3 (7f) in TMA of invasive breast cancers (BR20837a, Table 4) with grade 1, grade 2, or grade 3 samples. The colour legend in (7f) also applies to (7e).

FIGS. 8A-8H show WDR26 cytoplasmic localization correlates with breast cancer progression. 8a and 8b Quantification of the IHC scores of WDR26 (8a) and NCOA5 (8b) in the indicated tissue microarray of breast cancer progression. Data were analysed with two-way ANOVA. * vs. normal breast tissue, P<0.005. § vs. lobular hyperplasia, P<0.005. # vs. ductal carcinoma in situ, P<0.005. 8c and 8d Quantification of the IHC scores of WDR26 (8c) and NCOA5 (d) in the indicated TMA of invasive breast cancers. Data were analysed with two-way ANOVA. ¶ vs. grade 1, P<0.005. ♮ vs. grade 2, P<0.005. 8e Quantification of the IHC scores of TAp63 in the indicated tissue microarray of breast cancer progression. Data were analysed with two-way ANOVA. * vs. normal breast tissue, P<0.005. § vs. lobular hyperplasia, P<0.005. 8f Correlation of the IHC score and cellular distribution of WDR26 with the IHC score of TAp63 in the same tissue microarray as in (8e). 8g and 8h Kaplan-Meier curves of overall TCGA breast cancer 11 survival data showing the prognostic value of WDR26 in tumors with higher (8g) or lower (8h) than median levels of TROLL-3. P=0.02481 (g). P=0.18781 (h).

FIGS. 9A-9W show A Pan-cancer analysis reveals that localization of WDR26 in the cytoplasm drives cancer progression and metastatic disease. 9a Circos plot summarizing the expression of TROLL-2, TROLL-3, WDR26 and pAKT in TMA representing 378 cancers with progressive disease (Table 4). 9b-9g Correlation of the IHC score and cellular distribution of WDR26 with the ISH score of TROLL-3 in the indicated TMAs of ovarian cancer (9b), colon cancer (9c), lung adenocarcinoma (9d), lung squamous cell carcinoma (9e), and melanoma (9f and 9g). 9h Representative H&E stained cross sections of lung adenocarcinomas derived from H1299 cells infected with the indicated doxycycline-inducible shRNAs, and intrapulmonary injected in 6-week old athymic nu/nu mice (n=5 mice for all groups). The mice were fed doxycycline for the duration of the experiments to downregulate the lncRNA of interest. Black arrows indicate representative lung tumors. 9i Quantification of the lung adenocarcinomas described in (9h). n=5 mice for all groups, * vs. NT, P<0.005, two-tailed Student's t test. 9j Representative H&E stained cross sections of lung adenocarcinomas derived from H358 cells infected with the indicated doxycycline-inducible shRNAs, and intrapulmonary injected in 6-week old athymic nu/nu mice (n=5 mice for all groups). The mice were fed doxycycline for the duration of the experiments to downregulate the lncRNA of interest. Black arrows indicate representative lung tumors. 9k Quantification of the lung adenocarcinomas described in (j). n=5 mice for all groups, * vs. NT, P<0.005, two-tailed Student's t test. 9l Representative H&E stained cross sections of lung colonies derived from H1299 cells infected with the indicated doxycycline-inducible shRNAs, and injected in the heart of 6-week old athymic nu/nu mice (n=5 mice for all groups). The mice were fed doxycycline for the duration of the experiments to downregulate the lncRNA of interest. Black arrows indicate representative lung colonies. 9m Quantification of the lung colonies described in (9l). n=5 mice for all groups, * vs. NT, P<0.005, two-tailed Student's t test. 9n Representative H&E stained cross sections of lung colonies derived from H358 cells infected with the indicated doxycycline-inducible shRNAs, and injected in the heart of 6- week old athymic nu/nu mice (n=5 mice for all groups). The mice were fed doxycycline for the duration of the experiments to downregulate the lncRNA of interest. Black arrows indicate representative lung colonies. 9o Quantification of the lung colonies described in (n). n=5 mice for all groups, * vs. NT, P<0.005, two-tailed Student's t test. 9p Representative H&E stained cross sections of melanomas derived from A375 cells infected with the indicated doxycycline-inducible shRNAs, and injected subcutaneously in 6-week old athymic nu/nu mice (n=5 mice for all groups). The mice were fed doxycycline for the duration of the experiments to downregulate the lncRNA of interest. 9q Quantification of the melanomas described in (9p). n=5 mice for all groups, * vs. NT, P<0.005, two-tailed Student's t test. 9r Representative H&E stained cross sections of melanomas derived from Malme-3M cells infected with the indicated doxycycline-inducible shRNAs, and injected subcutaneously in 6-week old athymic nu/nu mice (n=5 mice for all groups). The mice were fed doxycycline for the duration of the experiments to downregulate the lncRNA of interest. 9s Quantification of the melanomas described in (r). n=5 mice for all groups, * vs. NT, P<0.005, two-tailed Student's t test. 9t Representative H&E stained cross sections of lung colonies derived from A375 cells infected with the indicated doxycycline-inducible shRNAs, and injected in the tail vein of 6-week old athymic nu/nu mice (n=5 mice for all groups). The mice were fed doxycycline for the duration of the experiments to downregulate the lncRNA of interest. Black arrows indicate representative lung colonies. 9u Quantification of the lung colonies described in (t). n=5 mice for all groups, * vs. NT, P<0.005, two-tailed Student's t test. 9v Representative H&E stained cross sections of lung colonies derived from Malme-3M cells infected with the indicated doxycycline-inducible shRNAs, and injected in the tail vein of 6-week old athymic nu/nu mice (n=5 mice for all groups). The mice were fed doxycycline for the duration of the experiments to downregulate the lncRNA of interest. Black arrows indicate representative lung colonies. 9w Quantification of the lung colonies described in (9v). n=5 mice for all groups, * vs. NT, P<0.005, two-tailed Student's t test.

FIGS. 10A-10F′ show a pan-cancer analysis reveals that localization of WDR26 in the cytoplasm drives cancer progression and metastatic disease. 10a, 10b, and 10c show quantification of the ISH scores of TROLL-2 (10a) and TROLL-3 (10b), and of the IHC score of WDR26 (10c) in the indicated TMA of ovarian cancer progression. Data were analysed with two-way ANOVA. * vs. normal ovarian tissue, P<0.005. § vs. serous adenocarcinoma, P<0.005. 10d Correlation of the IHC score and cellular distribution of WDR26 with the ISH score of TROLL-2 in the same ovarian cancer TMA as in (10a, 10b, and 10c). 10e, 10f, and 10g Quantification of the ISH scores of TROLL-2 (10e) and TROLL-3 (10f), and of the IHC score of WDR26 (10g) in the indicated TMA of colon cancer progression. Data were analysed with two-way ANOVA. * vs. normal colon tissue, P<0.005. § vs. polyp, P<0.005. # vs. adenoma, P<0.005. 10h Correlation of the IHC score and cellular distribution of WDR26 with the ISH score of TROLL-2 in the same colon cancer TMA as in (10e-10g). 10i, 10j, and 10k Quantification of the ISH scores of TROLL-2 (10i) and TROLL-3 (10j), and of the IHC score of WDR26 (10k) in the indicated TMA of lung adenocarcinoma progression. Data were analysed with two-way ANOVA. * vs. normal lung tissue, P<0.005. § vs. benign lesion, P<0.005. 10l Correlation of the IHC score and cellular distribution of WDR26 with the ISH score of TROLL-2 in the same lung adenocarcinoma TMA as in (10i-10k). 10m, 10n, and 10o Quantification of the ISH scores of TROLL-2 (10m) and TROLL-3 (10n), and of the IHC score of WDR26 (10o) in the indicated TMA of lung squamous cell carcinoma progression. Data were analysed with two-way ANOVA. * vs. normal lung tissue, P<0.005. § vs. benign lesion, P<0.005. # vs. squamous cell carcinoma, P<0.005. 10p Correlation of the IHC score and cellular distribution of WDR26 with the ISH score of TROLL-2 in the same lung squamous cell carcinoma TMA as in (10m-10o). 10q, 10r, and 10s Quantification of the ISH scores of TROLL-2 (10q) and TROLL-3 (10r), and of the IHC score of WDR26 (10s) in the indicated TMA of melanoma progression. Data were analysed with two-way ANOVA. * vs. benign nevus, P<0.005. 10t Correlation of the IHC score and cellular distribution of WDR26 with the ISH score of TROLL-2 in the same ovarian cancer TMA as in (10q-10s). 10u, 10v, and 10w Quantification of the ISH scores of TROLL-2 (10u) and TROLL-3 (10v) and of the IHC scores of WDR26 (10w) in the indicated TMA of melanoma. Data were analysed with two-way ANOVA. * vs. control cases, P<0.005. 10x Correlation of the IHC score and cellular distribution of WDR26 with the ISH score of TROLL-2 in the same ovarian cancer TMA as in (10u, 10v, 10w). 10y and 10z Kaplan-Meier curves of overall melanoma survival data showing the prognostic value of TROLL-2 (10y) and TROLL-3 (10z) in tumors of the indicated TMA with higher or lower than median levels of the considered lncRNA. P=0.0101 (y). P=0.0227 (z). 10a′,10b′ Kaplan-Meier curves of overall TCGA melanoma39 survival data showing the prognostic value of WDR26 in tumors with higher (10a′) or lower (10b′) than average levels of TROLL-3. P=0.02057 (a′). P=0.24559 (10b′). 10c′ Representative images of ISH for TROLL-2 (left panels) and TROLL-3 (middle panels) in tumors derived from H1299 cells shown in FIG. 9h,i. The LNA probe was detected and visualized via a chromogenic reaction (purple), while Nuclear Fast Red™ was used as a counterstain. A scramble LNA probe (right panel) was used as a negative control. 10d′ Representative images of ISH for TROLL-2 (left panels) and TROLL-3 (middle panels) in tumors derived from H358 cells shown in FIG. 9j,k. The LNA probe was detected and visualized via a chromogenic reaction (purple), while Nuclear Fast Red™ was used as a counterstain. A scramble LNA probe (right panel) was used as a negative control. 10e′ Representative images of ISH for TROLL-2 (left panels) and TROLL-3 (middle panels) in tumors derived from A375 cells shown in FIG. 9p,q. The LNA probe was detected and visualized via a chromogenic reaction (purple), while Nuclear Fast Red™ was used as a counterstain. A scramble LNA probe (right panel) was used as a negative control. 10f′ Representative images of ISH for TROLL-2 (left panels) and TROLL-3 (middle panels) in tumors derived from Malme-3M cells shown in FIG. 9r,s. The LNA probe was detected and visualized via a chromogenic reaction (purple), while Nuclear Fast Red™ was used as a counterstain. A scramble LNA probe (right panel) was used as a negative control.

FIGS. 11A-11J show TROLL-2 and TROLL-3 induce AKT phosphorylation through cytoplasmic localization of WDR26. 11a Quantification of the percentage of WDR26 localization in the nuclear (Nuc) and cytoplasmic (Cyt) fractions of CA1D cells transfected with the indicated siRNAs. Data are mean±SD and analysed with two-way ANOVA. n=3, * vs. siControl, P<0.05. 11b Representative western blot analysis of the coimmuno-precipitation of endogenous NOLC1 and WDR26 in MCF10A (left) and CA1D (right) cells transfected with the indicated constructs and siRNAs. 11c Representative western blot of WDR26 and NOLC1 localization in the nuclear (Nuc) and cytoplasmic (Cyt) fractions of MCF10A cells transfected with the indicated siRNAs. Histone H3 and HSP90 were used as controls for the nuclear and cytoplasmic fractions, respectively. 11d Quantification of cell migration of CA1D cells overexpressing either TROLL-2, TROLL-3, or empty vector, in combination with the indicated WDR26 constructs and transfected with the indicated siRNAs. Data are mean±SD and analysed with two-tailed Student's t test, n=3, * vs. pBabe Empty siControl, P<0.005. § vs. pBabe TROLL-2 siControl, P<0.005. # vs. pBabe TROLL-3 siControl, P<0.005. 11e Representative western blot in LPA-treated CA1D cells transfected with the indicated siRNAs and WDR26 constructs. 11f Representative western blot analysis of the coimmuno-precipitation of endogenous AKT and WDR26 in CA1D cells transfected with the indicated siRNAs. 11g, 11h, and 11i Representative H&E images (11g) and IHC for WDR26 (11h) and for pAKT (11i) in tumors derived from DCIS cells infected with the indicated constructs (n=5 mice for both groups). Black arrows indicate nuclei positive for WDR26. 11j Scheme describing the proposed mechanism of action of TROLL-2 and TROLL-3. In normal cells (e.g. MCF10A cells) the tumor and metastasis suppressor TAp63 inhibits the expression of TROLL-2 and TROLL-3, while NOLC1 interacts with WDR26 and promotes the accumulation of WDR26 in the nucleus. In cancer cells (e.g. CA1D cells), instead, mutant p53 inhibits TAp63, thus allowing for the expression of TROLL-2 and TROLL-3. These lncRNAs counteract the interaction between NOLC1 and WDR26, while promoting the binding of WDR26 to AKT. As a consequence, the PI3K/AKT pathway is activated and can sustain tumor formation and progression.

FIGS. 12A-12E″ TROLL-2 and TROLL-3 induce AKT phosphorylation through the regulation of the cytoplasmic localization of WDR26. 12a and 12b Representative western blot (12a) and quantification of the percentage (12b) of WDR26 localization in the nuclei (Nuc) and cytosols (Cyt) of the indicated cell lines. Histone H3 and HSP90 were used as controls, respectively. Data are mean±SD and analysed with two-way ANOVA. n=3, * vs. MCF10A, P<0.05. § vs. DCIS, P<0.005. 12c Representative western blot of WDR26 localization in the nuclear (Nuc) and cytoplasmic (Cyt) fractions of CA1D cells transfected with the indicated siRNAs. 12d Representative western blot of WDR26 localization in fractionated CA1D cells transfected with the indicated constructs. 12e-12g Quantification for EdU incorporation (12e), annexin V positivity (12f), and cell invasion (12g) of CA1D cells overexpressing either TROLL-2, TROLL-3, or the empty vector as a negative control, in combination with the indicated WDR26 constructs and transfected with the indicated siRNAs. Data are mean±SD and analysed with two-tailed Student's t test, n=3, * vs. pBabe Empty siControl, P<0.005. § vs. pBabe TROLL-2 siControl, P<0.005. # vs. pBabe TROLL-3 siControl, P<0.005. 12h,12i qRT-PCR for TROLL-2 (12h) and TROLL-3 (12i) of the CA1D cells treated as in (12e-12g). Data are mean±SD and analysed with two-tailed Student's t test, n=3, * vs. pBabe Empty siControl, P<0.005. 12j Representative western blot of WDR26 and FLAG in the CA1D cells treated as in (12e-12g). 12k Quantification of the ratio between pAKT (S473) and total AKT in western blots of LPA-treated CA1D cells transfected with the indicated siRNAs and WDR26 constructs. Data are mean±SD and analysed with two-tailed Student's t test, n=3, * vs. siControl—LPA treatment, P<0.005. 12l Quantification of the IHC scores of pAKT in the indicated TMA of breast cancer progression. Data were analysed with two-way ANOVA. * vs. normal breast tissue, P<0.005. § vs. lobular hyperplasia, P<0.005. # vs. ductal carcinoma in situ, P<0.005. 12m-12o Correlation of the IHC score of pAKT with the IHC score and cellular distribution of WDR26 (12m), and with the ISH score of TROLL-2 (12n) and TROLL-3 (12o) in the indicated TMA of breast cancer progression. p Quantification of the IHC scores of pAKT in the indicated TMA of invasive breast cancers. Data were analysed with two-way ANOVA. vs. grade 1, P<0.005. ♮ vs. grade 2, P<0.005. 12q-12s Correlation of the IHC score of pAKT with the IHC score and cellular distribution of WDR26 (12q), and with the ISH score of TROLL-2 (12r) and TROLL-3 (12s) in the indicated TMA of invasive breast cancers. 12t Quantification of the IHC scores of pAKT in the indicated TMA of invasive breast cancers. * vs. TNBC, P<0.005, Welchs t test. 12u-12w Correlation of the IHC score of pAKT with the IHC score and cellular distribution of WDR26 (12u), and with the ISH score of TROLL-2 (12v) and TROLL-3 (12w) in the indicated TMA of invasive breast cancers, whose cores are classified based on tumor grade and levels of PTEN as higher or lower than the median expression levels. 12x Quantification of the IHC score of pAKT in the indicated TMA of ovarian cancer progression. Data were analysed with two-way ANOVA. * vs. normal ovarian tissue, P<0.005. § vs. serous adenocarcinoma, P<0.005. 12y-12a′ Correlation of the IHC score of pAKT with the IHC score and cellular distribution of WDR26 (12y), and with the ISH score of TROLL-2 (12z) and TROLL-3 (12a′) in the indicated TMA of ovarian cancer. 12b′ Quantification of the IHC score of pAKT in the indicated TMA of colon cancer progression. Data were analysed with two-way ANOVA. * vs. normal colon tissue, P<0.005. § vs. polyp, P<0.005. # vs. adenoma, P<0.005. 12c′-12e′ Correlation of the IHC score of pAKT with the IHC score and cellular distribution of WDR26 (12c′), and with the ISH score of TROLL-2 (12d′) and TROLL-3 (12e′) in the indicated TMA of colon cancer. 12f′ Quantification of the IHC score of pAKT in the indicated TMA of lung adenocarcinoma progression. Data were analysed with two-way ANOVA. * vs. normal lung tissue, P<0.005. § vs. benign lesion, P<0.005. 12g′-12i′ Correlation of the IHC score of pAKT with the IHC score and cellular distribution of WDR26 (g′), and with the ISH score of TROLL-2 (12h′) and TROLL-3 (12i′) in the indicated TMA of lung adenocarcinoma. (12j′) Quantification of the IHC score of pAKT in the indicated TMA of lung squamous cell carcinoma progression. Data were analysed with two-way ANOVA. * vs. normal lung tissue, P<0.005. § vs. benign lesion, P<0.005. # vs. squamous cell carcinoma, P<0.005. 12k′-12m′ Correlation of the IHC score of pAKT with the IHC score and cellular distribution of WDR26 (12k′), and with the ISH score of TROLL-2 (12l′) and TROLL-3 (12m′) in the indicated TMA of lung squamous cell carcinoma. 12n′ Quantification of the IHC score of pAKT in the indicated TMA of melanoma. Data were analysed with two-way ANOVA. * vs. benign nevus, P<0.005. 12o′-12q′ Correlation of the IHC score of pAKT with the IHC score and cellular distribution of WDR26 (12o′), and with the ISH score of TROLL-2 (12p′) and TROLL-3 (12q′) in the indicated melanoma TMA. 12r′ Quantification of the IHC score of pAKT in the indicated TMA of melanoma progression. Data were analysed with two-way ANOVA. * vs. control cases, P<0.005. 12s′-12u′ Correlation of the IHC score of pAKT with the IHC score and cellular distribution of WDR26 (12s′), and with the ISH score of TROLL-2 (12t′) and TROLL-3 (12u′) in the indicated melanoma TMA. 12v′, 12w′ qRT-PCR of the indicated lncRNAs in the RNase-free CLIP-ed RNA interacting with endogenous WDR6 (12v′) and AKT (12w′) immunoprecipitated from CA1D cells transfected with the indicated siRNAs. Data are mean±SD, n=3, * vs. siControl, P<0.005, two-tailed Student's t test. 12x′,12y′ qRT-PCR of the indicated regions of TROLL-2 (12x′) and TROLL-3 (12y′) in the RNase-treated CLIP-ed RNA interacting with endogenous WDR6 immunoprecipitated from CA1D cells. Data are mean±SD, n=3. 12z′,12a″ Representative western blot (12z′) and quantification (12a″) of endogenous WDR26 pulled down from CA1D cells by streptavidin precipitation of the indicated in vitro synthesized and biotinylated full-length and deletion mutant lncRNAs. Data are mean±SD, n=3, * vs. the sense strand of the respective full-length lncRNA, P<0.005, two-tailed Student's t test. 12b″,12c″ Representative western blot (12b″) and quantification (12c″) of endogenous WDR26 pulled down from CA1D cells by streptavidin precipitation of the indicated in vitro synthesized and biotinylated sense and antisense lncRNAs. Data are mean±SD, n=3, * vs. the sense strand of the respective full-length lncRNA, P<0.005, two-tailed Student's t test.

FIGS. 13A-13E show the genomic structures of human TROLLs. FIG. 13A shows the genomic structure of human TROLL-1 (RP11-98G7.1). The isoforms targeted by antisense oligonucleotides (ENST00000441085.5 and ENST00000452465.1) are shown in the red box. FIG. 13B shows the genomic structure of human TROLL-2 (RPSAP52). The isoform targeted by antisense oligonucleotides (RPSAP52/NR_026825.2) is shown in the red box. FIG. 13C shows the genomic structure of human TROLL-3. all 4 transcripts were targeted in vivo via the shRNA and detected by ISH. NR_034110.1 is the transcript identified via RNA-seq and used for the overexpression assays. The region of NR_034110.1 interacting with WDR26 (467-482) is also present in NR_034108.1. The isoforms targeted by antisense oligonucleotides (TRAF3IP2-AS1/NR_034108.1, TRAF3IP2-AS1/NR_034109.1, TRAF3IP2-AS1/NR_034110.1, and TRAF3IP2-AS1/NR_034111.1) are shown in the red box. FIG. 13D shows the genomic structure of human TROLL-5. The isoform targeted by antisense oligonucleotides (LINC00514/NR_033861.1) is shown in the red box. FIG. 13E shows the genomic structure of human TROLL-7. The isoforms targeted by antisense oligonucleotides (MALAT1/NR_002819.4, MALAT1/NR_144567.1, and MALAT1/NR_144568.1) are shown in the red box. FIG. 13F shows the genomic structure of human TROLL-8. The isoform targeted by antisense oligonucleotides (ENST00000554568.1) is shown in the red box.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

As used herein “knockdown” or “knocking down” refers to any decrease or reduction in gene expression by genetic modification and/or a reagent. Gene expression does not have to be complete reduction or ablation (0% expression), which is referred to as a “knockout” but can include any individual, median, or average reduction in expression or activity in a statistically significant amount. Thus, the knockdown can be a reduction of a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, pro agents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Method Treating Cancer

One of the mechanisms by which mutant TP53 exerts its gain of function is through the inhibition of the p53 family member and p63 isoform, TAp63. We previously reported that TAp63 is a crucial tumor and metastasis suppressor. Mice lacking TAp63 (TAp63^{-/-}) develop highly metastatic tumors, with a large proportion being mammary adenocarcinomas that metastasize to the lung, liver, and brain. Moreover, deletion of TAp63 in murine and human mammary epithelial cells (MECs) triggers their transformation into tumor initiating cells, which give rise to mammary adenocarcinomas metastasizing to distant sites. The essential role of the tumor suppressive activity of TAp63 in human breast cancers is evident due to the inverse correlation of its expression with tumor grade.

The tumor and metastatic suppressive activity of TAp63 relies on the transcriptional regulation of gene expression and, until now, TAp63 has been shown to control the expression of protein-coding genes, including Dicer, and miRNAs. Here, we provide the first demonstration that TAp63 also governs the expression of long non-coding RNAs (lncRNAs), and notably that the levels and functional activities of two of these TAp63-regulated oncogenic lncRNAs or “TROLLs” correlate with the progression and tumor grade of a wide variety of human cancers. Using breast cancer as a model system and then extending our findings using a pan-cancer approach including xenograft mouse models, TCGA datasets, and 723 clinical cases, we provide molecular and functional evidence that the tumorigenic and metastatic potential of these lncRNAs is mediated by one of their interacting proteins, WDR26. The cytoplasmic localization of WDR26, which we found to be typical of advanced cancers, is controlled by TROLL-2 and TROLL-3 via the shuttling protein NOLC1 and is required for the pro-oncogenic and metastatic activities of WDR26, including the interaction with AKT and the induction of its activating phosphorylation on Ser473. The physical and functional interaction between the two TROLLs and WDR26 is particularly significant for basal-like breast cancers and melanomas, where high levels of these lncRNAs as well as high levels of TROLL-3 and WDR26 correlate with poor prognosis. Taken together, our findings identify a novel mechanism for the activation of the AKT pathway through TAp63-regulated lncRNAs (TROLLs) and pave the way for more effective therapies against metastatic cancers with alterations in TP53 and hyperactivation of the PI3K/AKT pathway.

In one aspect, disclosed herein are methods of treating, inhibiting, reducing, ameliorating and/or preventing a cancer, cancer recurrence, and/or metastasis (such as, for example, a breast cancer (including, but not limited to triple negative breast cancer), lung cancer (including, but not limited to adenocarcinomas and squamous cell carcinomas), ovarian (including, but not limited to serous and non-serous adenocarcinomas), colon, or melanoma) in a subject comprising knocking down expression of one or any combination of two or more of TROLL-1, TROLL-2, TROLL-3, TROLL-4, TROLL-5, TROLL-6, TROLL-7, TROLL-8, and/or TROLL-9 in the subject. For example, the cancer can be treated by knocking down expression of TROLL-1; TROLL-2; TROLL-3; TROLL-4; TROLL-5; TROLL-6; TROLL-7; TROLL-8; TROLL-9; TROLL-1 and TROLL-2, TROLL-1 and TROLL-3; TROLL-1 and TROLL-4; TROLL-1 and TROLL-5; TROLL-1 and TROLL-6; TROLL-1 and TROLL-7; TROLL-1 and TROLL-8; and/or TROLL-1 and TROLL-9; TOLLS 2 and 3; TROLLs 2 and 4; TROLLs 2 and 5; TROLLs 2 and 6; TROLLs 2 and 7; TROLLs 2 and 8; TROLLs 2 and 9; TROLLs 3 and 4; TROLLs 3 and 5; TROLLs 2 and 6; TROLLs 3 and 7; TROLLs 3 and 8; TROLLs 3 and 9; TROLLs 4 and 5; TROLLs 4 and 6; TROLLs 4 and 7; TROLLs 4 and 8; TROLLs 4 and 9; TROLLs 5 and 6; TROLLs 5 and 7; TROLLs 5 and 8; TROLLs 5 and 9; TROLLs 6 and 7; TROLLs 6 and 8; TROLLs 6 and 9; TROLLs 7 and 8; TROLLs 7 and 9; TROLLs 8 and 9; TROLLs 1, 2, and 3; TROLLs 1, 2, and 4; TROLLs 1, 2, and 5; TROLLs 1, 2, and 6; TROLLs 1, 2, and 7; TROLLs 1, 2, and 8; TROLLs 1, 2, and 9; TROLLs 1, 3, and 4; TROLLs 1, 3, and 5; TROLLs 1, 3, and 6; TROLLs 1, 3, and 7; TROLLs 1, 3, and 8; TROLLs 1, 3, and 9; TROLLs 1, 4, and 5; TROLLs 1, 4, and 6; TROLLs 1, 4, and 7; TROLLs 1, 4, and 8; TROLLs 1, 4, and 9; TROLLs 1, 5, and 6; TROLLs 1, 5, and 7; TROLLs 1, 5, and 8; TROLLs 1, 5, and 9; TROLLs 1, 6, and 7; TROLLs 1, 6, and 8; TROLLs 1, 6, and 9; TROLLs 1, 7, and 8; TROLLs 1, 7, and 9; TROLLs 1, 8, and 9; TROLLs 2, 3, and 4; TROLLs 2, 3, and 5; TROLLs 2, 3, and 6; TROLLs 2, 3, and 7; TROLLs 2, 3, and 8; TROLLs 2, 3, and 9; TROLLs 2, 4, and 5; TROLLs 2, 4, and 6; TROLLs 2, 4, and 7; TROLLs 2, 4, and 8; TROLLs 2, 4, and 9; TROLLs 2, 4, and 6; TROLLs 2, 4, and 7; TROLLs 2, 4, and 8; TROLLs 2, 4, and 9; TROLLs 2, 5, and 6; TROLLs 2, 5, and 7; TROLLs 2, 5, and 8; TROLLs 2, 5, and 9; TROLLs 2, 6, and 7; TROLLs 2, 6, and 8; TROLLs 2, 6, and 9; TROLLs 2, 7, and 8; TROLLs 2, 7, and 9; TROLLs 2, 8, and 9; TROLLs 3, 4, and 5; TROLLs 3, 4, and 6; TROLLs 3, 4, and 7; TROLLs 3, 4, and 8; TROLLs 3, 4, and 9; TROLLs 3, 5, and 6; TROLLs 3, 5, and 7; TROLLs 3, 5, and 8; TROLLs 3, 5, and 9; TROLLs 3, 6, and 7; TROLLs 3, 6, and 8; TROLLs 3, 6, and 9; TROLLs 3, 7, and 8; TROLLs 3, 7, and 9; TROLLs 3, 8, and 9; TROLLs 4, 5, and 6; TROLLs 4, 5, and 7; TROLLs 4, 5, and 8; TROLLs 4, 5, and 9; TROLLs 4, 6, and 7; TROLLs 4, 6, and 8; TROLLs 4, 6, and 9; TROLLs 4, 7, and 8; TROLLs 4, 7, and 9; TROLLs 4, 8, and 9; TROLLs 5, 6, and 7; TROLLs 5, 6, and 8; TROLLs 5, 6, and 9; TROLLs 5, 7, and 8; TROLLs 5, 7, and 9; TROLLs 5, 8, and 9; TROLLs 6, 7, and 8; TROLLs 6, 7, and 9; TROLLs 6, 8, and 9; TROLLs 7, 8, and 9; TROLLs 1, 2, 3, and 4; TROLLs, 1, 2, 3, and 5; TROLLs 1, 2, 3, and 6; TROLLs 1, 2, 3, and 7; TROLLs 1, 2, 3, and 8; TROLLs 1, 2, 3, and 9; TROLLs, 1, 2, 4, and 5; TROLLs 1, 2, 4, and 6; TROLLs 1, 2, 4, and 7; TROLLs 1, 2, 4, and 8; TROLLs 1, 2, 4, and 9; TROLLs 1, 2, 5, and 6; TROLLs 1, 2, 5, and 7; TROLLs 1, 2, 5, and 8; TROLLs 1, 2, 5, and 9; TROLLs 1, 2, 6, and 7; TROLLs 1, 2, 6, and 8; TROLLs 1, 2, 6, and 9; TROLLs 1, 2, 7, and 8; TROLLs 1, 2, 7, and 9; TROLLs 1, 2, 8, and 9; TROLLs 1, 3, 4, and 5; TROLLs 1, 3, 4, and 6; TROLLs 1, 3, 4, and 7; TROLLs 1, 3, 4, and 8; TROLLs 1, 3, 4, and 9; TROLLs 1, 3, 5, and 6; TROLLs 1, 3, 5, and 7; TROLLs 1, 3, 5, and 8; TROLLs 1, 3, 5, and 9; TROLLs 1, 3, 6, and 7; TROLLs 1, 3, 6, and 8; TROLLs 1, 3, 6, and 9; TROLLs 1, 3, 7, and 8; TROLLs 1, 3, 7, and 9; TROLLs 1, 3, 7, and 9; TROLLs 1, 4, 5, and 6; TROLLs, 1, 4, 5, and 7; TROLLs 1, 4, 5, and 8; TROLLs 1, 4, 5, and 9; TROLLs 1, 4, 6, and 7; TROLLs 1, 4, 6, and 8; TROLLs 1, 4, 6, and 9; TROLLs 1, 4, 7, and 8; TROLLs 1, 4, 7, and 9; TROLLs 1, 4, 8, and 9; TROLLs 1, 5, 6, and 7; TROLLs 1, 5, 6, and 8; TROLLs 1, 5, 6, and 9; TROLLs 1, 5, 7, and 8; TROLLs 1, 5, 7, and 9; TROLLs 1, 5, 8, and 9; TROLLs 1, 6, 7, and 8; TROLLs 1, 6, 7, and 9; TROLLs 1, 6, 8, and 9; TROLLs 1, 7, 8, and 9; TROLLs 2, 3, 4, and 5; TROLLs 2, 3, 4, and 6; TROLLs 2, 3, 4, and 7; TROLLs 2, 3, 4, and 8; TROLLs 2, 3, 4, and 9; TROLLs 2, 3, 5, and 6; TROLLs 2, 3, 5, and 7; TROLLs 2, 3, 5, and 8; TROLLs 2, 3, 5, and 9; TROLLs 2, 3, 6, and 7; TROLLs 2, 3, 6, and 8; TROLLs 2, 3, 6, and 9; TROLLs 2, 3, 7, and 8; TROLLs 2, 3, 7, and 9; TROLLs 2, 3, 7, and 9; TROLLs 2, 4, 5, and 6; TROLLs, 2, 4, 5, and 7; TROLLs 2, 4, 5, and 8; TROLLs 2, 4, 5, and 9; TROLLs 2, 4, 6, and 7; TROLLs 2, 4, 6, and 8; TROLLs 2, 4, 6, and 9; TROLLs 2, 4, 7, and 8; TROLLs 2, 4, 7, and 9; TROLLs 2, 4, 8, and 9; TROLLs 2, 5, 6, and 7; TROLLs 2, 5, 6, and 8; TROLLs 2, 5, 6, and 9; TROLLs 2, 5, 7, and 8; TROLLs 2, 5, 7, and 9; TROLLs 2, 5, 8, and 9; TROLLs 2, 6, 7, and 8; TROLLs 2, 6, 7, and 9; TROLLs 2, 6, 8, and 9; TROLLs 2, 7, 8, and 9; TROLLs 3, 4, 5, and 6; TROLLs, 3, 4, 5, and 7; TROLLs 3, 4, 5, and 8; TROLLs 3, 4, 5, and 9; TROLLs 3, 4, 6, and 7; TROLLs 3, 4, 6, and 8; TROLLs 3, 4, 6, and 9; TROLLs 3, 4, 7, and 8; TROLLs 3, 4, 7, and 9; TROLLs 3, 4, 8, and 9; TROLLs 3, 5, 6, and 7; TROLLs 3, 5, 6, and 8; TROLLs 3, 5, 6, and 9; TROLLs 3, 5, 7, and 8; TROLLs 3, 5, 7, and 9; TROLLs 3, 5, 8, and 9; TROLLs 3, 6, 7, and 8; TROLLs 3, 6, 7, and 9; TROLLs 3, 6, 8, and 9; TROLLs 3, 7, 8, and 9; TROLLs 4, 5, 6, and 7; TROLLs 4, 5, 6, and 8; TROLLs 4, 5, 6, and 9; TROLLs 4, 5, 7, and 8; TROLLs 4, 5, 7, and 9; TROLLs 4, 5, 8, and 9; TROLLs 4, 6, 7, and 8; TROLLs 4, 6, 7, and 9; TROLLs 4, 6, 8, and 9; TROLLs 4, 7, 8, and 9; TROLLs 5, 6, 7, and 8; TROLLs 5, 6, 7, and 9; TROLLs 5, 6, 8, and 9; TROLLs 5, 7, 8, and 9; TROLLs 6, 7, 8, and 9; TROLLs 1, 2, 3, 4, and 5, TROLLs 1, 2, 3, 4, and 6; TROLLs 1, 2, 3, 4, and 7; TROLLs 1, 2, 3, 4, and 8; TROLLs 1, 2, 3, 4, and 9; TROLLs 1, 2, 3, 5, and 6; TROLLs 1, 2, 3, 5, and 7; TROLLs 1, 2, 3, 5, and 8; TROLLs 1, 2, 3, 5, and 9; TROLLs 1, 2, 3, 6, and 7; TROLLs 1, 2, 3, 6, and 8; TROLLs 1, 2, 3, 6, and 9; TROLLs 1, 2, 3, 7, and 8; TROLLs 1, 2, 3, 7, and 9; TROLLs 1, 2, 3, 8, and 9; TROLLs 1, 2, 4, 5, and 6; TROLLs 1, 2, 4, 5, and 7; TROLLs 1, 2, 4, 5, and 8; TROLLs 1, 2, 4, 5, and 9; TROLLs 1, 2, 4, 6, and 7; TROLLs 1, 2, 4, 6, and 8; TROLLs 1, 2, 4, 6, and 9; TROLLs 1, 2, 4, 7, and 8; TROLLs 1, 2, 4, 7, and 9; TROLLs 1, 2, 4, 8, and 9; TROLLs 1, 2, 5, 6, and 7; TROLLs 1, 2, 5, 6, and 8; TROLLs 1, 2, 5, 6, and 9; TROLLs 1, 2, 5, 7, and 8; TROLLs 1, 2, 5, 7, and 9; TROLLs 1, 2, 5, 8, and 9; TROLLs 1, 2, 6, 7, and 8; TROLLs 1, 2, 6, 7, and 9; TROLLs 1, 2, 6, 8, and 9; TROLLs 1, 2, 7, 8, and 9; TROLLs 1, 3, 4, 5, and 6; TROLLs 1, 3, 4, 5, and 7; TROLLs 1, 3, 4, 5, and 8; TROLLs 1, 3, 4, 5, and 9; TROLLs 1, 3, 4, 6, and 7; TROLLs 1, 3, 4, 6, and 8; TROLLs 1, 3, 4, 6, and 9; TROLLs 1, 3, 4, 7, and 8; TROLLs 1, 3, 4, 7, and 9; TROLLs 1, 3, 4, 8, and 9; TROLLs 1, 3, 5, 6, and 7; TROLLs 1, 3, 5, 6, and 8; TROLLs 1, 3, 5, 6, and 9; TROLLs 1, 3, 5, 7, and 8; TROLLs 1, 3, 5, 7, and 9; TROLLs 1, 3, 5, 8, and 9; TROLLs 1, 3, 6, 7, and 8; TROLLs 1, 3, 6, 7, and 9; TROLLs 1, 3, 6, 8, and 9; TROLLs 1, 3, 7, 8, and 9; TROLLs 1, 4, 5, 6, and 7; TROLLs 1, 4, 5, 6, and 8; TROLLs 1, 4, 5, 6, and 9; TROLLs 1, 4, 5, 7, and 8; TROLLs 1, 4, 5, 7, and 9; TROLLs 1, 4, 5, 8, and 9; TROLLs 1, 4, 6, 7, and 8; TROLLs 1, 4, 6, 7, and 9; TROLLs 1, 4, 6, 8, and 9; TROLLs 1, 4, 7, 8, and 9; TROLLs 1, 5, 6, 7, and 8; TROLLs 1, 5, 6, 7, and 9; TROLLs 1, 5, 6, 8, and 9; TROLLs 1, 5, 7, 8, and 9; TROLLs 1, 6, 7, 8, and 9; TROLLs 2, 3, 4, 5, and 6; TROLLs 2, 3, 4, 5, and 7; TROLLs 2, 3, 4, 5, and 8; TROLLs 2, 3, 4, 5, and 9; TROLLs 2, 3, 4, 6, and 7; TROLLs 2, 3, 4, 6, and 8; TROLLs 2, 3, 4, 6, and 9; TROLLs 2, 3, 4, 7, and 8; TROLLs 2, 3, 4, 7, and 9; TROLLs 2, 3, 4, 8, and 9; TROLLs 2, 3, 5, 6, and 7; TROLLs 2, 3, 5, 6, and 8; TROLLs 2, 3, 5, 6, and 9; TROLLs 2, 3, 5, 7, and 8; TROLLs 2, 3, 5, 7, and 9; TROLLs 2, 3, 5, 8, and 9; TROLLs 2, 3, 6, 7, and 8; TROLLs 2, 3, 6, 7, and 9; TROLLs 2, 3, 6, 8, and 9; TROLLs 2, 3, 7, 8, and 9; TROLLs 2, 4, 5, 6, and 7; TROLLs 2, 4, 5, 6, and 8; TROLLs 2, 4, 5, 6, and 9; TROLLs 2, 4, 5, 7, and 8; TROLLs 2, 4, 5, 7, and 9; TROLLs 2, 4, 5, 8, and 9; TROLLs 2, 4, 6, 7, and 8; TROLLs 2, 4, 6, 7, and 9; TROLLs 2, 4, 6, 8, and 9; TROLLs 2, 4, 7, 8, and 9; TROLLs 2, 5, 6, 7, and 8; TROLLs 2, 5, 6, 7, and 9; TROLLs 2, 5, 6, 8, and 9; TROLLs 2, 5, 7, 8, and 9; TROLLs 2, 6, 7, 8, and 9; TROLLs 3, 4, 5, 6, and 7; TROLLs 3, 4, 5, 6, and 8; TROLLs 3, 4, 5, 6, and 9; TROLLs 3, 4, 5, 7, and 8; TROLLs 3, 4, 5, 7, and 9; TROLLs 3, 4, 5, 8, and 9; TROLLs 3, 4, 6, 7, and 8; TROLLs 3, 4, 6, 7, and 9; TROLLs 3, 4, 6, 8, and 9; TROLLs 3, 4, 7, 8, and 9; TROLLs 3, 5, 6, 7, and 8; TROLLs 3, 5, 6, 7, and 9; TROLLs 3, 5, 6, 8, and 9; TROLLs 3, 5, 7, 8, and 9; TROLLs 3, 6, 7, 8, and 9; TROLLs 4, 5, 6, 7, and 8; TROLLs 4, 5, 6, 7, and 9; TROLLs 4, 5, 6, 8, and 9; TROLLs 4, 5, 7, 8, and 9; TROLLs 4, 6, 7, 8, and 9; TROLLs 5, 6, 7, 8, and 9; TROLLs 1, 2, 3, 4, 5, and 6; TROLLs 1, 2, 3, 4, 5, and 7; TROLLs 1, 2, 3, 4, 5, and 8; TROLLs 1, 2, 3, 4, 5, and 9; TROLLs 1, 2, 3, 4, 6, and 7; TROLLs 1, 2, 3, 4, 6, and 8; TROLLs 1, 2, 3, 4, 6, and 9; TROLLs 1, 2, 3, 4, 7, and 8; TROLLs 1, 2, 3, 4, 7, and 9; TROLLs 1, 2, 3, 4, 8, and 9; TROLLs 1, 2, 3, 5, 6, and 7; TROLLs 1, 2, 3, 5, 6, and 8; TROLLs 1, 2, 3, 5, 6, and 9; TROLLs 1, 2, 3, 5, 7, and 8; TROLLs 1, 2, 3, 5, 7, and 9; TROLLs 1, 2, 3, 5, 8, and 9; TROLLs 1, 2, 3, 6, 7, and 8; TROLLs 1, 2, 3, 6, 7, and 9; TROLLs 1, 2, 3, 6, 8, and 9; TROLLs, 1, 2, 3, 7, 8, and 9; TROLLs 1, 2, 4, 5, 6, and 7; TROLLs 1, 2, 4, 5, 6, and 8; TROLLs 1, 2, 4, 5, 6, and 9; TROLLs 1, 2, 4, 5, 7, and 8; TROLLs 1, 2, 4, 5, 7, and 9; TROLLs 1, 2, 4, 5, 8, and 9; TROLLs 1, 2, 4, 6, 7, and 8; TROLLs 1, 2, 4, 6, 7, and 9; TROLLs 1, 2, 4, 6, 8, and 9; TROLLs, 1, 2, 4, 7, 8, and 9; TROLLs 1, 2, 5, 6, 7, and 8; TROLLs 1, 2, 5, 6, 7, and 9; TROLLs 1, 2, 5, 6, 8, and 9; TROLLs, 1, 2, 5, 7, 8, and 9; TROLLs 1, 2, 5, 6, 8, and 9; TROLLs, 1, 2, 6, 7, 8, and 9; TROLLs 1, 3, 4, 5, 6, and 7; TROLLs 1, 3, 4, 5, 6, and 8; TROLLs 1, 3, 4, 5, 6, and 9; TROLLs 1, 3, 4, 5, 7, and 8; TROLLs 1, 3, 4, 5, 7, and 9; TROLLs 1, 3, 4, 5, 8, and 9; TROLLs 1, 3, 4, 6, 7, and 8; TROLLs 1, 3, 4, 6, 7, and 9; TROLLs 1, 3, 4, 6, 8, and 9; TROLLs, 1, 3, 4, 7, 8, and 9; TROLLs 1, 3, 5, 6, 7, and 8; TROLLs 1, 3, 5, 6, 7, and 9; TROLLs 1, 3, 5, 6, 8, and 9; TROLLs, 1, 3, 5, 7, 8, and 9; TROLLs 1, 3, 5, 6, 8, and 9; TROLLs, 1, 3, 6, 7, 8, and 9; TROLLs 1, 4, 5, 6, 7, and 8; TROLLs 1, 4, 5, 6, 7, and 9; TROLLs 1, 4, 5, 6, 8, and 9; TROLLs, 1, 4, 5, 7, 8, and 9; TROLLs 1, 4, 5, 6, 8, and 9; TROLLs, 1, 4, 6, 7, 8, and 9; TROLLs 1, 5, 6, 7, 8, and 9; TROLLs 2, 3, 4, 5, 6, and 7; TROLLs 2, 3, 4, 5, 6, and 8; TROLLs 2, 3, 4, 5, 6, and 9; TROLLs 2, 3, 4, 5, 7, and 8; TROLLs 2, 3, 4, 5, 7, and 9; TROLLs 2, 3, 4, 5, 8, and 9; TROLLs 2, 3, 4, 6, 7, and 8; TROLLs 2, 3, 4, 6, 7, and 9; TROLLs 2, 3, 4, 6, 8, and 9; TROLLs, 2, 3, 4, 7, 8, and 9; TROLLs 2, 3, 5, 6, 7, and 8; TROLLs 2, 3, 5, 6, 7, and 9; TROLLs 2, 3, 5, 6, 8, and 9; TROLLs, 2, 3, 5, 7, 8, and 9; TROLLs 2, 3, 5, 6, 8, and 9; TROLLs, 2, 3, 6, 7, 8, and 9; TROLLs 2, 4, 5, 6, 7, and 8; TROLLs 2, 4, 5, 6, 7, and 9; TROLLs 2, 4, 5, 6, 8, and 9; TROLLs, 2, 4, 5, 7, 8, and 9; TROLLs 2, 4, 5, 6, 8, and 9; TROLLs, 2, 4, 6, 7, 8, and 9; TROLLs 2, 5, 6, 7, 8, and 9; TROLLs 3, 4, 5, 6, 7, and 8; TROLLs 3, 4, 5, 6, 7, and 9; TROLLs 3, 4, 5, 6, 8, and 9; TROLLs, 3, 4, 5, 7, 8, and 9; TROLLs 3, 4, 5, 6, 8, and 9; TROLLs, 3, 4, 6, 7, 8, and 9; TROLLs 3, 5, 6, 7, 8, and 9; TROLLs 4, 5, 6, 7, 8, and 9; TROLLs 1, 2, 3, 4, 5, 6, and 7; TROLLs 1, 2, 3, 4, 5, 6, and 8; TROLLs 1, 2, 3, 4, 5, 6, and 9; TROLLs, 1, 2, 3, 4, 5, 7, and 8; TROLLs, 1, 2, 3, 4, 5, 7, and 9; TROLLs, 1, 2, 3, 4, 5, 8, and 9; TROLLs, 1, 2, 3, 4, 6, 7, and 8; TROLLs, 1, 2, 3, 4, 6, 7, and 9; TROLLs, 1, 2, 3, 4, 6, 8, and 9; TROLLs, 1, 2, 3, 4, 7, 8, and 9; TROLLs, 1, 2, 3, 5, 6, 7, and 8; TROLLs, 1, 2, 3, 5, 6, 7, and 9; TROLLs, 1, 2, 3, 5, 6, 8, and 9; TROLLs, 1, 2, 3, 5, 7, 8, and 9; TROLLs, 1, 2, 3, 6, 7, 8, and 9; TROLLs, 1, 2, 4, 5, 6, 7, and 8; TROLLs, 1, 2, 4, 5, 6, 7, and 9; TROLLs, 1, 2, 4, 5, 6, 8, and 9; TROLLs, 1, 2, 4, 5, 7, 8, and 9; TROLLs, 1, 2, 4, 6, 7, 8, and 9; TROLLs 1, 2, 5, 6, 7, 8, and 9; TROLLs, 1, 3, 4, 5, 6, 7, and 8; TROLLs, 1, 3, 4, 5, 6, 7, and 9; TROLLs, 1, 3, 4, 5, 6, 8, and 9; TROLLs, 1, 3, 4, 5, 7, 8, and 9; TROLLs, 1, 3, 4, 6, 7, 8, and 9; TROLLs 1, 3, 5, 6, 7, 8, and 9; TROLLs 1, 4, 5, 6, 7, 8, and 9; TROLLs, 2, 3, 4, 5, 6, 7, and 8; TROLLs, 2, 3, 4, 5, 6, 7, and 9; TROLLs, 2, 3, 4, 5, 6, 8, and 9; TROLLs, 2, 3, 4, 5, 7, 8, and 9; TROLLs, 2, 3, 4, 6, 7, 8, and 9; TROLLs 2, 3, 5, 6, 7, 8, and 9; TROLLs 2, 4, 5, 6, 7, 8, and 9; TROLLs 3, 4, 5, 6, 7, 8, and 9; TROLLs 1, 2, 3, 4, 5, 6, 7, and 8; TROLLs 1, 2, 3, 4, 5, 6, 7, and 9; TROLLs 1, 2, 3, 4, 5, 6, 8, and 9; TROLLs 1, 2, 3, 4, 5, 7, 8, and 9; TROLLs, 1, 2, 3, 4, 6, 7, 8, and 9; TROLLs, 1, 2, 3, 5, 6, 7, 8, and 9; TROLLs, 1, 2, 4, 5, 6, 7, 8, and 9; TROLLs, 1, 3, 4, 5, 6, 7, 8, and 9; TROLLs, 2, 3, 4, 5, 6, 7, 8, and 9; and/or TROLLs, 1, 2, 3, 4, 5, 6, 7, 8, and 9.

In some aspects, the expression of one or any combination of two or more of TROLL-1, TROLL-2, TROLL-3, TROLL-4, TROLL-5, TROLL-6, TROLL-7, TROLL-8, and/or TROLL-9 is knocked down (i.e., a reduction or decrease in expression) by a therapeutic that targets RNA. Examples of RNA-targeted therapeutics are well-known in the art, including, but not limited to antisense oligonucleotides, siRNA, shRNA, ribozymes, transcription activator-like effector nucleases (TALEN), zinc finger nucleases (ZFNs) and/or clustered regularly interspaced short palindromic repeats/associated (CRISPR/Cas) nucleases. As noted herein Table 2 provides siRNA that can be used in the disclosed methods. Accordingly, disclosed herein are methods of treating, inhibiting, reducing, decreasing, ameliorating, and/or preventing a cancer and/or metastasis in a subject, wherein the expression of TROLL-1 is knocked down by targeting the ENST00000441085.5 and/or ENST00000452465.1 isoform of TROLL-1 (for example by administering an siRNA as set forth in SEQ ID NO: 10, SEQ ID NO 11, and/or SEQ ID NO: 12); wherein the expression of TROLL-2 is knocked down by targeting the RPSAP52/NR_026825.2 isoform of TROLL-2 (for example by administering an siRNA as set forth in SEQ ID NO: 13, SEQ ID NO 14, and/or SEQ ID NO: 15); wherein the expression of TROLL-3 is knocked down by targeting the TRAF3IP2-AS1/NR_034108.1, TRAF3IP2-AS1/NR_034109.1, TRAF3IP2-AS1/NR_034110.1, and/or TRAF3IP2-AS1/NR_034111.1 isoform of TROLL-3 (for example by administering an siRNA as set forth in SEQ ID NO: 16, SEQ ID NO 17, and/or SEQ ID NO: 18); wherein TROLL-4 expression is knocked down by targeting the NR_015410 isoform of TROLL-4 (for example by administering an siRNA as set forth in SEQ ID NO: 19, SEQ ID NO 20, and/or SEQ ID NO: 21); wherein the expression of TROLL-5 is knocked down by targeting the LINC00514/NR_033861.1 isoform of TROLL-5 (for example by administering an siRNA as set forth in SEQ ID NO: 22, SEQ ID NO 23, and/or SEQ ID NO: 24); wherein TROLL-6 expression is knocked down by targeting the RP11-138I1.3 isoform of TROLL-6 (for example by administering an siRNA as set forth in SEQ ID NO: 25, SEQ ID NO 26, and/or SEQ ID NO: 27); wherein the expression of TROLL-7 is knocked down by targeting the MALAT1/NR_002819.4, MALAT1/NR_144567.1, and/or MALAT1/NR_144568.1 isoform of TROLL-7 (for example by administering an siRNA as set forth in SEQ ID NO: 28, SEQ ID NO 29, and/or SEQ ID NO: 30); wherein the expression of TROLL-8 is knocked down by targeting the ENST00000554568.1 isoform of TROLL-8 (for example by administering an siRNA as set forth in SEQ ID NO: 31, SEQ ID NO 32, and/or SEQ ID NO: 33); and/or wherein TROLL-9 expression is knocked down by targeting the RP11-126K1.6 isoform of TROLL-9 ((for example by administering an siRNA as set forth in SEQ ID NO: 34, SEQ ID NO 35, and/or SEQ ID NO: 36). As any combination of TROLLs may be inhibited it is understood that treatment of a cancer can comprise the administration of any one or combination of 2, 3, 4, 5, 6, 7, 8, 9, 10, 11, 12, 13, 14, 15, 16, 17, 18, 19, 20, 21, 22, 23, 24, 25, 26, or all 27 of SEQ ID NOs 10-36.

The disclosed compositions can be used to treat, inhibit, reduce, and/or prevent any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. For example, the cancer can be a Tap63 regulated cancer such as breast cancer (but not limited to triple negative breast cancer), lung cancer (including, but not limited to adenocarcinomas and squamous cell carcinomas), ovarian (including, but not limited to serous and non-serous adenocarcinomas), liver cancer, colon cancer, or melanoma. In some instances the cancer comprises a p53 mutation. It is understood and herein contemplated that the opportunity to treat a cancer using the methods disclosed herein may not present itself in a subject until after a caner has established or has become metastatic. In one aspect, disclosed herein are methods of treating, inhibiting, reducing, ameliorating and/or preventing a cancer wherein the cancer is already established in the subject prior to the initiation of treatment.

The disclosed treatment methods are not limited to the use of the disclosed siRNAs or antisense oligonucleotides, shRNA, ribozymes, transcription activator-like effector nucleases (TALEN), zinc finger nucleases (ZFNs) and/or clustered regularly interspaced short palindromic repeats/associated (CRISPR/Cas) nucleasesor to knock down expression of TROLL-1, TROLL-2, TROLL-3, TROLL-4, TROLL-5, TROLL-6, TROLL-7, TROLL-8, and/or TROLL-9 and can include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Where an EGFR splice variant isoform is not detected, the treatment methods can include or further include checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016). Where the presence of an EGFR splice variant isoform is detected the treatment regimen implemented does not include an immune checkpoint blockade inhibitor. It is understood and herein recognized that the presence of an EGFR splice variant isoform does not necessarily indicate that the cancer is resistant to all immune checkpoint blockade inhibitors. In one aspect, the detection of the EGFR splice variant isoform indicates resistance to PD-1, PD-L1, PD-12, CRLA-4, IDO, B7-H3, B7-H4, TIM3, or LAG-3. In one aspect, the detection of the EGFR splice variant isoform indicates resistance to PD-L1. Thus, when resistance is only to a particular form of immune checkpoint blockade inhibition (such as, for example PD-L1), other immune checkpoint blockade inhibitors can still be used. Additionally, the disclosed treatment regiments can employ the use of immunotherapies such as CAR T cells, CAR NK cells, TILs, and MILs.

1. Pharmaceutical Carriers/Delivery of Pharmaceutical Products

As noted herein, Table 2 provides 35 siRNA that can be used to knockdown expression of TROLL-1, TROLL-2, TROLL-3, TROLL-4, TROLL-5, TROLL-6, TROLL-7, TROLL-8, and/or TROLL-9. In one aspect, disclosed herein are siRNA comprising any of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36. Also disclosed herein are compositions comprising any one or combination of 2, 3, 4, 5, 6, 7, 8, 9, 10, 11, 12, 13, 14, 15, 16, 17, 18, 19, 20, 21, 22, 23, 24, 25, 26, or all 27 of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36.

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Pan-Cancer Analysis Reveals TAp63-Regulated Oncogenic lncRNAs (TROLLs) that Promote Cancer Progression Through AKT Activation

a) Results

(1) Identification of TAp63-Regulated Long Non-Coding RNAs (lncRNAs) (TROLLs) in Human Breast Cancer Progression

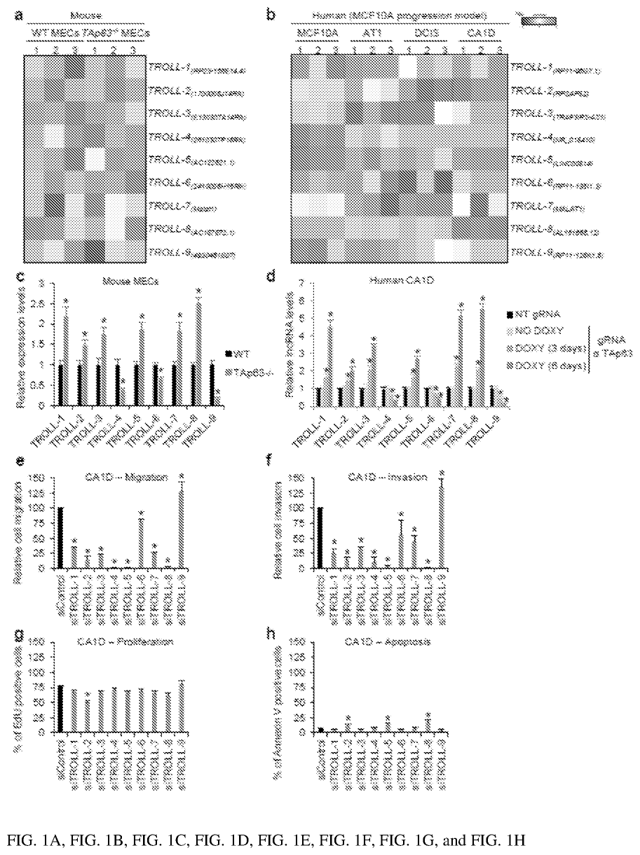

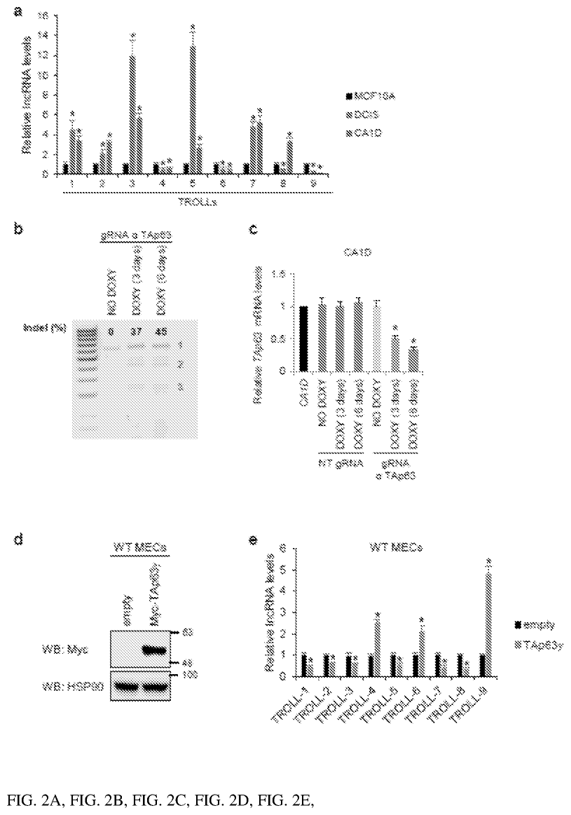

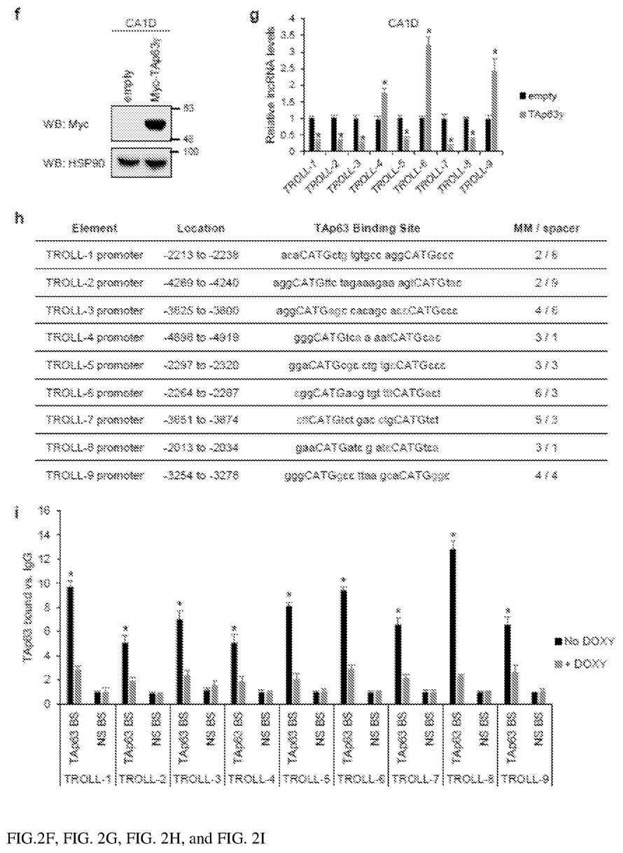

TP53 missense mutations are the most frequent genetic alterations in breast cancers11 and inactivate the tumor and metastasis suppressor TAp633. Importantly, we have previously shown that loss of TAp63 leads to the onset of highly metastatic mammary adenocarcinomas to distant sites, making this a faithful mouse model of human metastatic breast cancers5. By performing RNA-sequencing (RNA-seq) analysis of wild-type (WT) and TAp63−/− mammary epithelial cells (MECs)6, we found that TAp63−/− MECs contained 591 long non-coding RNAs (lncRNAs) that were differentially expressed compared to WT MECs. To determine whether these mouse lncRNAs had human orthologs involved in breast cancer formation and progression, we used a locus conservation approach12 to compare the differentially expressed mouse lncRNAs to human lncRNAs differentially expressed in the MCF10A breast cancer progression model13, 14, comprised of 4 cell lines: i) MCF10A (normal mammary epithelial cells); ii) AT1 (transformed); iii) DCIS (tumorigenic); and iv) CA1D (metastatic cells). This strategy allowed us to identify 9 TAp63-regulated oncogenic lncRNAs or TROLLs in mouse and human breast cancer progression (FIG. 1a,b). Importantly, one of the identified TROLLs was MALAT1, previously demonstrated to promote different metastatic tumor types in humans15, 16, 17, including breast cancer where high levels of this lncRNA correlate with higher risk of relapse and reduced overall survival18, 19, 20. We performed qRT-PCR and found that 6 TROLLs were upregulated and 3 were downregulated in TAp63−/− MECs compared with WT MECs (FIG. 1c), and similarly their human orthologs were differentially expressed in the MCF10A breast cancer progression model cell lines (FIG. 2a). To assess whether expression of these 9 human orthologs is TAp63-dependent, we targeted TAp63 in the metastatic mammary adenocarcinoma cell line, CA1D, via a doxycycline-inducible CRISPR/Cas9 system21. After 6 days of induction, we achieved a 45% cleavage efficiency in the TAp63 locus (FIG. 2b) with a concurrent downregulation of TAp63 mRNA levels (FIG. 2c). Reduced levels of TAp63 were associated with the upregulation of 6 lncRNAs and the downregulation of 3 lncRNAs (FIG. 1d), similar to what we observed for their murine orthologs in TAp63−/− vs. WT MECs. Conversely, the overexpression of TAp63 in both WT MECs and in CA1D cells was associated to the opposite change in the expression of these 9 TROLLs (FIG. 2d-g). To verify whether the 9 human lncRNAs are bona fide direct target genes of TAp63, we analysed their respective promoters for possible p63 response elements and found putative TAp63 binding sites in all of these promoters (FIG. 2h). Chromatin immunoprecipitation (ChIP) experiments revealed TAp63 recruitment to these sites (FIG. 2i). These results indicate that TAp63 directly binds these promoters to transcriptionally regulate the expression of these 9 conserved lncRNAs.

We previously demonstrated that TAp63 suppresses metastatic cancer by regulating a transcriptome that halts cancer migration and invasion5, 6. To determine whether these 9 conserved lncRNAs are involved in these biological properties associated with metastasis, CA1D cells were transfected with siRNAs targeting these lncRNAs, and cell migration and invasion were assessed. Down-regulation of these lncRNAs severely affected the migratory and invasive potential of CA1D cells (FIG. 1e,f) while having little effect on cell proliferation and survival (FIG. 1g,h). Indeed, only the silencing of one lncRNA (TROLL-2) decreased cell proliferation assessed by 5-ethynyl-2′-deoxyuridine (EdU) incorporation (FIG. 1g), and the silencing of 3 of them (TROLL-2, TROLL-5, and TROLL-8) caused a slight yet significant increase in the percentage of apoptotic cells, as quantified by annexin V staining (FIG. 1h). These findings indicate that TAp63 directly regulates the expression of conserved lncRNAs, which in turn are required for the efficient migration and invasion of mammary adenocarcinomas.

(2) TROLL-2 and TROLL-3 Promote the Tumorigenic and Metastatic Potential of Human Breast Cancers

To assess the relevance of the identified lncRNAs in human breast cancers, we decided to focus our attention on two lncRNAs, TROLL-2 and TROLL-3, since they are the only two among the 9 TROLLs to be divergent, i.e. lncRNAs transcribed on the opposite strand compared to a nearby protein-coding gene and generally sharing similar functions in line with the guilt-by-association principle22, 23. Intriguingly, their respective antisense protein coding genes (HMGA2 for TROLL-2 and TRAF3IP2, also known as ACT1, for TROLL-3) are both known oncogenes supporting tumor growth and dissemination24, 25. To assess whether these two lncRNAs were expressed in human invasive breast cancers, we performed in situ hybridization (ISH) for TROLL-2 and TROLL-3 in a breast cancer tissue microarray (TMA) with 45 samples including normal breast tissue, lobular hyperplasia, DCIS, and invasive breast cancer biopsies. Notably, we found that the levels of both lncRNAs were undetectable in normal breast tissue and increased with breast cancer progression with the highest levels observed in invasive breast cancer samples (FIG. 5a,b). Interestingly, the expression of TROLL-2 and TROLL-3 positively correlated in these samples (FIG. 4a), indicating that they both may be important markers of breast cancer progression. Since the metastatic potential of invasive breast cancers is affected by their tumor grade26, we analysed the levels of TROLL-2 and TROLL-3 based on this feature. We found that the levels of both lncRNAs were higher in grade 3 compared to grade 1 and 2 tumors in two distinct TMAs, which included 77 (Biomax TMA) and 154 (Dundee TMA) invasive breast cancers, respectively (FIG. 4b-e). Since in the latter TMA the mutational status of TP53 had been reported27 and given that mutated forms of p53 are known to inhibit TAp63 functions3, we analysed the correlation between the presence of mutant p53 and the levels of TROLL-2 and TROLL-3. Notably, we found that both lncRNAs were more abundant in tumors bearing mutant p53 (FIG. 3c,d), indicating that TROLL-2 and TROLL-3 may be critical breast cancer progression markers in p53 mutant cases. In line with the fact that mutations in TP53 are more frequent in basal-like tumors (mainly consisting of the so-called triple-negative cases28) compared to the other breast cancer subtypes11, we found that the levels of both lncRNAs were highest in triple-negative breast cancers (TNBCs) in a TMA including 68 breast cancers (FIG. 4f,g). Since in this TMA the overall survival data of the patients had been reported29, we stratified the patients based on the levels of TROLL-2 and TROLL-3 and found that high levels of either lncRNA correlated with reduced overall survival of these breast cancer patients (FIG. 3e,f). Together, these data indicate that TROLL-2 and TROLL-3 are prognostic factors and may be markers of breast cancer progression. Given that the expression of TROLL-2 and TROLL-3 are elevated in breast tumors and correlate with breast cancer progression, we asked whether TROLL-2 and TROLL-3 are required for tumor formation in vivo using two different orthotopic xenograft models of breast cancer, CA1D and MDA MB-231 cells, with the latter growing in vivo due to the inhibition of TAp63 by mutant p5330, 31. To efficiently reduce the levels of TROLL-2 and TROLL-3, CA1D and MDA MB-231 cells were infected with a doxycycline-inducible shRNA targeting either lncRNA. These cells were then orthotopically injected in the mammary fat pads of nude mice and the mice were fed with doxycycline containing food throughout the experiment. At the end point, we found that the volume of the tumors expressing the shRNAs targeting the lncRNAs were 3 to 5 times smaller than the tumors derived from the respective control cells (FIG. 3g-j). Downregulation of TROLL-2 or TROLL-3 in these tumors was confirmed by ISH staining, which showed a clear reduction in the levels of either lncRNA in the tumors expressing the respective shRNA compared to the strong cytoplasmic signals detected in the control tumors (FIG. 4h,i). Next, we assessed the possible role of TROLL-2 and TROLL-3 in lung colonization in vivo. To accomplish this, the same cells used for the orthotopic injection experiments were injected in the tail vein of nude mice, and lung colonization was assessed after 5 weeks (MDA MB-231 cells) or 10 weeks (CA1D cells) of doxycycline treatment to downregulate TROLL-2 and TROLL-3. Inspection of the lungs revealed the presence of tumor cells covering an average area of 7% (CA1D control cells) and 15% (MDA MB-231 control cells) of the lungs (FIG. 3k-n). In contrast, downregulation of either TROLL-2 or TROLL-3 severely impaired the lung colonization potential of these breast cancer cells and less than 2% of the lung area was colonised (FIG. 3k-n). Together, these data show that downregulation of TROLL-2 and TROLL-3 severely impairs the tumorigenic and metastatic potential of two different orthotopic xenograft models of breast cancer and suggest that the breast cancer phenotype observed in a context where TAp63 is inhibited is in part mediated by TROLL-2 and TROLL-3.

(3) TROLL-2 and TROLL-3 Mediate their Tumorigenic and Metastatic Activities Through WDR26

LncRNAs are known to affect different molecular processes, including chromatin remodelling, alternative splicing, and miRNA activity, and their effects are achieved by the interaction with specific proteins that ultimately act as their effectors32, 33. To identify such interacting proteins, we in vitro transcribed the only reported transcript of TROLL-2 (NR_026825.2) and the longest transcript of TROLL-3 (NR_034110.1), which is the isoform that we identified as differentially expressed in the MCF10A breast cancer progression model. These in vitro transcribed RNAs where then used to probe a protein microarray array34 containing ˜9,400 human recombinant full-length proteins. This led to the identification of 60 putative interactors of TROLL-2 and 19 for TROLL-3. Seven of these proteins were found as common for both lncRNAs (FIG. 5a,b). Since downregulation of TROLL-2 and TROLL-3 showed a similar phenotype in vivo (i.e. reduced formation of mammary tumors and lung colonization), we determined whether any of these 7 common putative interactors could act as effectors of TROLL-2 and TROLL-3. To do this, we overexpressed either TROLL-2 or TROLL-3 in CA1D cells and observed an increase in cell migration and invasion in vitro (FIG. 5c,d). Concomitantly, CA1D cells expressing either TROLL-2 or TROLL-3 were transfected with siRNAs targeting the 7 identified proteins individually (FIG. 6a-i) to establish if the absence of any of them could prevent the increased cell migration and invasion due to TROLL-2 and TROLL-3 overexpression. We found that downregulation of two proteins (WDR26 and NCOA5) hindered the migration and invasion potential of CA1D cells overexpressing TROLL-2 or TROLL-3 (FIG. 5c,d). A similar but less intense effect was observed in cell proliferation (FIG. 6j), while only downregulation of WDR26 showed a modest effect on apoptosis (FIG. 6k). Since WDR26 and NCOA5 were required for the oncogenic activities of TROLL-2 and TROLL-3, we further validated the interactions between these two proteins and the two lncRNAs. In vitro transcribed and biotinylated TROLL-2 and TROLL-3 efficiently pulled down both WDR26 and NCOA5 (FIG. 5e). These results indicate that both TROLL-2 and TROLL-3 interact with WDR26 and NCOA5, which mediate the common pro-oncogenic activities of these two lncRNAs.

(4) WDR26 Cytoplasmic Localization Correlates with Breast Cancer Progression

Because the expression of TROLL-2 and TROLL-3 positively correlate with breast cancer progression, we assessed the expression levels of their 2 interacting proteins, WDR26 and NCOA5 in a TMA of breast cancer progression with 45 samples, comprising normal breast tissue, lobular hyperplasia, DCIS, and invasive breast cancer biopsies. Interestingly, we found that the cellular localization of WDR26 was mainly nuclear in normal breast tissue and lobular hyperplasia, while in the advanced phases of the disease (i.e. DCIS and invasive ductal carcinoma samples) WDR26 localization was almost exclusively cytoplasmic (FIG. 7a,b). In addition, we found that the expression of both WDR26 and NCOA5 increased over the progression of breast cancer (FIG. 8a,b) and were enriched in highly aggressive and poorly differentiated grade 3 tumors compared with grade 1 and 2 cases (FIG. 8c,d). Importantly, when we examined the localization of WDR26 in the TMA of breast cancer progression based on the ISH score of TROLL-2 and TROLL-3, we found that high levels of these two lncRNAs correlated with high levels and cytoplasmic localization of WDR26 (FIG. 7c,d). This positive correlation was also observed in breast tumors stratified based on tumor grade (FIG. 7e,f). Notably, the higher levels and cytoplasmic localization of WDR26 inversely correlated with the IHC score of TAp63, whose levels are higher in normal breast tissue compared to the other groups, in lines with our previous findings5 (FIG. 8e,f). Together, these data indicate that during breast cancer progression, the levels of TAp63 are reduced while those of TROLL-2 and TROLL-3 increase, and this correlates with the cytoplasmic localization of WDR26.

Because the lncRNAs are prognostic factors in breast cancer progression (see FIG. 3e,f) and their levels correlate with those of WDR26, we assessed whether the combination of either lncRNA with WDR26 may be prognostic in breast cancer. To do this, overall TCGA breast cancer survival data11 were analysed based on the levels of the two lncRNAs and WDR26. While the combination of TROLL-2 and WDR26 did not reach statistical significance, we found that WDR26 is prognostic in basal-like tumors with high levels of TROLL-3, but not with low levels of TROLL-3 (FIG. 8e,f). These findings suggest that when TROLL-3 levels are high, which correlate with the cytoplasmic localization of WDR26, breast cancers are more aggressive and associated with reduced overall survival.

(5) A Pan-Cancer Analysis Reveals that Localization of WDR26 in the Cytoplasm Drives Cancer Progression and Metastatic Disease

We next asked whether expression of TROLL-2 and TROLL-3 correlated with cytoplasmic WDR26 more broadly across other aggressive human cancers by performing a pan-cancer analysis. We performed ISH for TROLL-2 and TROLL-3 and IHC for WDR26 in 378 tumor specimens, consisting of 51 ovarian (Biomax TMA, including serous and non-serous adenocarcinomas), 73 colon (Biomax TMA), 55 lung (Biomax TMA, including adenocarcinomas and squamous cell carcinomas), and 199 melanoma cases (Biomax TMA and Moffitt TMA). In line with our observations in breast cancer, all the malignant tumor types assessed had increased levels of TROLL-2, TROLL-3 and WDR26 compared to normal tissue and benign lesions (FIG. 9a). Importantly, increased expression of the two TROLLs and cytoplasmic WDR26 correlated with higher tumor grade (FIG. 9b-g and FIG. 10a-x), indicating that the regulation of WDR26 cytoplasmic localization by TROLL-2 and TROLL-3 may be a universal mechanism in the progression to aggressive disease.

Given that one of the melanoma TMAs (Moffitt TMA) contains the overall survival data of the patients, we stratified the patients based on the levels of TROLL-2 and TROLL-3 and found that high levels of either lncRNA correlated with reduced overall survival of these melanoma patients (Supplementary FIG. 5y,z). Additionally, given that WDR26 is prognostically important in basal-like tumors with high expression of TROLL-3 (see FIG. 8g,h), we verified whether these factors may also be prognostic in other tumor types. To assess this, overall TCGA ovarian cancer35, colon cancer36, lung adenocarcinoma37, lung squamous carcinoma38, and melanoma39 survival data were analyzed based on the expression levels of TROLL-3 and WDR26. Among the tested cancer types, we found that WDR26 is prognostic in melanomas with high levels of TROLL-3 (FIG. 10a′,b′). Altogether, our results indicate that the levels of TROLL-2 and TROLL-3 and the cytoplasmic location of WDR26 are markers of cancer progression in several human tumor types, and that these factors are prognostic in melanoma.

These results prompted us to verify whether TROLL-2 and TROLL-3 are required for the formation and progression of these tumor types in vivo. To accomplish this, we first utilised two orthotopic models of lung adenocarcinoma (H1299 and H358 cells), where TROLL-2 and TROLL-3 were downregulated via doxycycline-inducible shRNAs. These cells were injected either in the lungs or in the hearts of nude mice to assess their in vivo ability to form primary lung adenocarcinomas40 or secondary lung colonies41, respectively. While the control cells successfully developed lung adenocarcinomas (FIG. 9h-k) and lung colonies (FIG. 9l-o), downregulation of either TROLL-2 or TROLL-3 strongly impaired the formation of both (FIG. 9h-o). The downregulation of TROLL-2 and TROLL-3 in the lung adenocarcinomas was confirmed via ISH (FIG. 10c′-d′). Next, we utilized two xenograft models of melanoma (A37542 and Malme-3M43 cells), which were infected with doxycycline-inducible shRNAs to downregulate either TROLL-2 or TROLL-3. The cells were then injected subcutaneously or via tail vein in nude mice to test their ability to generate melanomas and lung colonies, respectively. As observed in both the breast cancer and the lung adenocarcinoma models, downregulation of either lncRNA significantly reduced the tumorigenic (FIG. 9p-s) and metastatic (FIG. 9t-w) potential of both melanoma cell lines. The effect of the shRNAs in downregulating the respective lncRNA was confirmed by ISH (FIG. 10e′-f′). Taken together, these results demonstrate that TROLL-2 and TROLL-3 are markers of cancer progression and are necessary for tumor and metastasis formation in multiple cancer types.

(6) TROLL-2 and TROLL-3 Induce AKT Phosphorylation Through the Regulation of the Cytoplasmic Localization of WDR26

Given the correlation between high expression of TROLL-2 and TROLL-3 and the cytoplasmic localization of WDR26 in invasive human cancers, we hypothesised that these two lncRNAs may promote the cytoplasmic localization of WDR26 to promote metastasis. To test this, we first determined whether WDR26 is localized in the cytoplasm in the MCF10A breast cancer progression model. Indeed, by examining the expression of WDR26 in the nuclear and cytoplasmic fractions of the MCF10A progression model, we found that WDR26 was primarily nuclear in MCF10A cells (representing normal epithelial cells) and cytoplasmic in the metastatic CA1D cells (FIG. 12a,b). Further, we transfected CA1D cells with siRNAs targeting TROLL-2 and TROLL-3 and assessed WDR26 cellular localization through fractionation. Importantly, we found that downregulation of either lncRNA decreased the pool of WDR26 present in the cytoplasmic fraction promoting its nuclear localization (FIG. 11a and FIG. 12c). To investigate how WDR26 localization is regulated, we generated WDR26 mutants devoid of either its nuclear localization signal (herein indicated as WDR26-ΔNLS) or its nuclear export signal (herein indicated as WDR26-ΔNES) (FIG. 12d) and looked for proteins that differentially interact with these two mutants in CA1D cells by performing a liquid chromatography-mass spectrometry (LC-MS/MS) analysis. Intriguingly, the top-ranking protein interacting exclusively with WDR26-ΔNES was NOLC1 (also known as Nopp140) (Supplementary table 6), a shuttling protein 44 known to affect the localization of several proteins45, 46, 47. Therefore, we tested whether the interaction between endogenous NOLC1 and WDR26 occurs in the MCF10A progression model and if it is affected by TROLL-2 and TROLL-3. Notably, we found that NOLC1 interacts with WDR26 more efficiently in MCF10A cells, where WDR26 is mainly nuclear and the levels of TROLL-2 and TROLL-3 are lower, than in CA1D cells, where WDR26 is mainly cytoplasmic and the levels of TROLL-2 and TROLL-3 are higher (FIG. 11b). Additionally, this interaction is regulated by the two lncRNAs. Indeed, the overexpression of both lncRNAs in MCF10A cells counteracts the binding between NOLC1 and WDR26, while the downregulation of both lncRNAs in CA1D cells promotes it (FIG. 11b). Since NOLC1 was shown to control the localization of multiple proteins45, 46, 47, we then assessed whether it also affects WDR26 localization. To this aim, we downregulated NOLC1 in MCF10A cells via siRNA and found that reduced levels of NOLC1 increased the pool of WDR26 present in the cytoplasm (FIG. 11c). Together, these data suggest that TROLL-2 and TROLL-3 promote WDR26 cytoplasmic localization by preventing its interaction with the shuttling protein NOLC1.

Since reduced expression of TROLL-2 and TROLL-3 (see FIG. 1e,f) and of WDR26 (FIG. 5c,d) impair cell migration and invasion of CA1D cells, we tested whether the different cellular distribution of WDR26 can affect the migratory and invasive potential of these cells. To do this, we transfected CA1D cells with an siRNA targeting the 3′UTR of WDR26, thus specifically affecting only endogenous WDR26, and overexpressed WDR26-ΔNLS or WDR26-ΔNES. Notably, the overexpression of WDR26-ΔNLS was able to rescue the cell migration and invasion of WDR26 deficient CA1D cells (FIG. 11d and FIG. 12e-j). On the contrary, WDR26-ΔNES showed no appreciable difference in migration or invasion compared to cells treated with empty vector (FIG. 11d and FIG. 12e-j), indicating that cytoplasmic WDR26, but not nuclear WDR26, functions downstream of TROLL-2 and TROLL-3 and plays a crucial role in cancer progression. WDR26 is a scaffold protein reported to promote phosphorylation of AKT, a crucial hub of cell survival pathways, and its downstream biological effects48. Therefore, we assessed whether cytoplasmic localization of WDR26 had an effect on AKT phosphorylation. To do so, the same CA1D cells expressing WDR26 mutants used for the cell migration and invasion experiments were serum starved for 24 h and AKT phosphorylation triggered via a lysophosphatidic acid (LPA) treatment, known to be WDR26-dependent48. Notably, only cytoplasmic WDR26 (i.e. WDR26-ΔNLS) was able to promote AKT phosphorylation to a similar extent as WT WDR26, while nuclear WDR26 (i.e. WDR26-ΔNES) failed to do so (FIG. 11e and FIG. 12k), indicating that the cytoplasmic location of WDR26 is required for phosphorylation and induction of AKT signalling.

AKT phosphorylation has been shown to rely on the efficient interaction between PI3K and AKT mediated by WDR2648. Since we found that TROLL-2 and TROLL-3 control the cellular localization of WDR26 which in turn promotes AKT phosphorylation, we tested whether the two lncRNAs are required for AKT and WDR26 to form complexes. To do this, AKT and WDR26 were individually immunoprecipitated in CA1D cells transfected with siRNAs targeting either lncRNA. Downregulation of either TROLL-2 or TROLL-3 strongly impaired the interaction between AKT and WDR26, ultimately leading to a decrease in AKT phosphorylation levels (FIG. 11f). To investigate the in vivo consequences of TROLL-2 and TROLL-3 on WDR26 cellular localization and AKT phosphorylation, we infected the tumorigenic DCIS cells either with an empty vector as a negative control or with both pBabe TROLL-2 and pBabe TROLL-3, and then injected these cells orthotopically in the mammary fat pads of nude mice. Notably, while the DCIS cells gave rise to well-differentiated comedo-like tumors as previously reported49, DCIS cells overexpressing both lncRNAs produced poorly differentiated tumors (FIG. 11g) reminiscent of invasive ductal carcinomas (IDC). Importantly, this

phenotypic change was accompanied both by cytoplasmic localization of WDR26 (FIG. 11h), and by an increased amount of phosphorylated AKT (pAKT) (FIG. 11i). Taken together, these results indicate that TROLL-2 and TROLL-3 promote the cytoplasmic localization of WDR26 and its interaction with AKT, which in turn trigger the activation of the AKT pathway to mediate the oncogenic and invasive activities of these two lncRNAs.

Given the correlation between high expression of TROLL-2 and TROLL-3, WDR26 localization, and AKT phosphorylation in DCIS derived tumors, we tested whether such correlation was also present in the breast cancer TMAs that we assessed for the ISH of TROLL-2 and TROLL-3 and the IHC of WDR26. Notably, we found that pAKT levels increased with breast cancer progression and tumor grade, and positively correlated with the levels of TROLL-2 and TROLL-3 and with the cytoplasmic localization of WDR26 (FIG. 9a and FIG. 12l-w). Next, we used a pan-cancer approach to determine whether TROLL-2 and TROLL-3 activated AKT through WDR26 in a broader cancer context. We assessed the correlation between pAKT levels and either the expression of the two lncRNAs or the localization of WDR26 in TMAs of ovarian cancer, colon cancer, lung adenocarcinoma, lung squamous cell carcinoma, and melanoma. Importantly, in all the tested TMAs we found that high expression of pAKT positively correlated with expression of TROLL-2 and TROLL-3 and cytoplasmic expression of WDR26 (FIG. 9a and FIG. 12x-u′), thus indicating that this novel interplay that we have identified is relevant in multiple tumor types.

To characterize this interplay occurring between TROLL-2, TROLL-3, WDR26, and AKT, we performed a cross-linking immunoprecipitation and qRT-PCR (CLIP-qPCR) assay50, 51 in CA1D cells. We found that not only endogenous WDR26 directly interacts with both lncRNAs (FIG. 12v′), but that it is also required for the interaction between these lncRNAs and AKT (FIG. 12w′). To map the region in the two lncRNAs necessary for their binding to WDR26, we repeated the CLIP-qPCR assay in the presence of RNAse treatment50, 51. This approach allowed us to narrow down the portions of the two lncRNAs involved in the interaction with WDR26 to nucleotides 403-627 in TROLL-2 and nucleotides 360-603 in TROLL-3 (FIG. 12x′,y′). Notably, these two regions contain a common sequence (corresponding to nucleotides 522-538 on TROLL-2 and 467-482 on TROLL-3). To assess if this common sequence is mediating the interaction between the lncRNAs and WDR26, we generated deletion mutants (TROLL-2 □522-538 and TROLL-3 □467-482) and tested their ability to interact with endogenous WDR26 in CA1D cells. Compared to the full-length lncRNAs, deletion of these regions significantly impaired the binding of these lncRNAs to WDR26 (FIG. 12z′,a″), thus indicating that endogenous WDR26 binds to TROLL-2 and TROLL-3 in CA1D cells and that this interaction is mediated by a common sequence present in both lncRNAs. To determine whether WDR26 and the two lncRNAs form a single complex, we performed a pull-down assay in CA1D cells transfected with siRNA against either lncRNA. Interestingly, we found that reduced levels of either lncRNA impaired the interaction between WDR26 and the remaining lncRNA (FIG. 12b″,c″), thus suggesting that both lncRNAs interact at the same time with WDR26 possibly forming a trimeric complex. In line with this hypothesis, we found that the overexpression of one lncRNA cannot rescue the decrease in cell migration and invasion due to the downregulation of the other lncRNA (FIG. 12d″,e″). Taken together, our results demonstrate that TROLL-2 and TROLL-3 are markers of cancer progression and regulate the cytoplasmic localization of WDR26 via NOLC1 by forming a trimeric complex with WDR26 that leads to the phosphorylation of AKT and the subsequent induction of oncogenic properties crucial for tumor progression and metastasis formation (FIG. 11j).

b) Discussion

Long non-coding RNAs (lncRNAs) constitute an ever-growing category of functional RNA species known to impinge on all hallmarks of cancer33, 52, 53. Here, we report the identification of two TAp63-regulated oncogenic lncRNAs or “TROLLs”, by using both genetically engineered and xenograft mouse models as well as a pan-cancer approach, including the analysis of 723 clinical cases and TCGA overall survival datasets. We found that the expression of two TAp63-regulated lncRNAs, TROLL-2 and TROLL-3, is prognostic in breast cancers and melanomas and positively correlates with the progression of many cancer types, including breast, ovarian, colon, lung adenocarcinoma and lung squamous cell carcinoma, and melanoma. Mechanistically, we demonstrated using xenograft mouse models that TROLL-2 and TROLL-3 promote the formation and the progression of different human cancers, including metastatic mammary adenocarcinomas, lung adenocarcinomas, and melanomas. These oncogenic effects are achieved via the scaffold protein WDR26, whose localization is controlled by the two lncRNAs through the shuttling protein NOLC1. In the cytoplasm, the trimeric complex formed by WDR26, TROLL-2 and TROLL-3, leads to the phosphorylation of AKT on Ser473 and the subsequent pro-oncogenic and metastatic effects of the AKT pathway (FIG. 6j).

Our mouse-human cross species analysis, comparing the TAp63 metastatic mammary adenocarcinoma mouse model5 and the well characterised MCF10A model of human breast cancer progression13, 14, allowed us to identify a group of 9 TAp63-regulated lncRNAs, which include TROLL-2 and TROLL-3. Both lncRNAs are relevant for metastatic breast cancer. In particular, our observation that the overexpression of TROLL-2 or TROLL-3 in DCIS cells is sufficient to promote tumor dedifferentiation and invasive breast cancer indicate that TROLL-2 and TROLL-3 are strong drivers of tumor progression. Notably, the levels of both lncRNAs are higher in invasive breast cancers expressing mutant p53, a potent inhibitor of TAp63 function3, thus making our findings relevant for a large percentage of breast cancer patients (37% of all cases, up to 80% in the basal-like subtype11) and possibly for other tumor types harbouring TP53 mutations.

We have demonstrated that the tumorigenic activities of TROLL-2 and TROLL-3 are mediated by one of their common interacting proteins, WDR26, which is a scaffold protein transducing the PI3K signalling pathway48. WDR26 contains a WD40 domain, which has been reported to act as a non-canonical RNA binding domain54, 55. Indeed, several proteins have been shown to interact with lncRNAs via their WD40 domains, as in the case of the association between LRRK2 and LINK-A56 and between LLGL2 and MAYA57. Thus, we speculate that the WD40 domain of WDR26 may mediate its binding to TROLL-2 and TROLL-3. We have shown that both lncRNAs bind to endogenous WDR26 forming a trimeric complex and that this interaction is mediated by a nucleotide sequence present in both lncRNAs. This complex is important for the localization of WDR26. Indeed, these lncRNAs prevent WDR26 from binding to the shuttling protein NOLC1 and being sequestered into the nucleus. Instead, the trimeric complex including WDR26 and both lncRNAs localizes in the cytoplasm, where it triggers AKT phosphorylation on Ser473 which is essential to activate the AKT pathway58. Importantly, our findings in cellular systems were corroborated not only in orthotopic mouse models but also in tissue microarrays of a broad range of human cancers, indicating that expression of TROLL-2 and TROLL-3 represents a novel mechanism of AKT activation in cancer progression. AKT is a pivotal hub funnelling cell growth stimuli and controlling multiple cellular functions, including cell survival, proliferation, and migration59. Given the high frequency of oncogenic activating mutations affecting the PI3K/AKT pathway in human tumors60, our data provide preclinical rationale suggesting that the inactivation of TROLL-2 and TROLL-3, the nuclear sequestration of WDR26, or interfering with the interaction between the TROLLs, WDR26 and AKT might be effective in halting cancer progression and resistance to AKT targeted therapy. In conclusion, by utilizing mouse models of a broad array of aggressive cancers and by performing a pan-cancer analysis, we have identified novel biomarkers associated with cancer progression: two TAp63-regulated oncogenic lncRNAs or “TROLLs” and one of their common interacting proteins, WDR26. The physical and functional interaction of TROLL-2 and TROLL-3 with WDR26, as well as its cytoplasmic localization during cancer progression, support the potential use of these factors as markers of cancer progression and as possible predictors of the efficacy for AKT inhibitor-based therapies, and their suitability as therapeutic targets for the development of novel therapies to treat a broad range of metastatic cancers.

c) Methods

(1) Cell Lines and Culture Conditions.

The MCF10A progression model cell lines (MCF10A, AT1, DCIS, and CA1D) were obtained from the Karmanos Cancer Institute (Detroit, Mich.) and cultured in Dulbecco's modified Eagle's medium (DMEM)/F12 (1:1) media containing 5% horse serum, 10 μg/mL insulin, 20 ng/mL epidermal growth factor, and 500 ng/mL hydrocortisone. Primary mouse mammary gland epithelial cells were isolated from 10-week-old WT and TAp63−/− female mice as previously described6, and cultured in DMEM/F12 (1:1) media containing the same components used for MCF10A cells. The human breast carcinoma cells (MDA MB-231) and lung cancer cells (H1299 and H358) were maintained in culture as previously reported10, 27, 61. The human melanoma cell lines, A375 and Malme-3m, were cultured in DMEM media containing 10% and 20% foetal bovine serum, respectively. All cultured cells were mycoplasma negative.

(2) Cross-Species Analysis of Coding and Non-Coding RNAs Using RNA Sequencing.

The mouse RNA-Seq data6 was mapped using TopHat62 against the mouse genome build UCSC mm10 and quantified using Cufflinks63 against the Gencode64 mouse gene reference. Data was quantile normalized. We analysed previously published RNA-Seq transcriptomic profiles of the human isogenic MCF10A breast cancer progression mode113, corresponding to normal breast tissue (MCF10A), atypia (AT1), ductal carcinoma in situ (DCIS) and invasive breast cancer (CA1D). The RNA-Seq data was mapped using TopHat62 against the human genome build UCSC hg19 and quantified using Cufflinks63 against a combined reference comprised of Gencode65 and two lncRNA catalogues66, 67. Using bedtools we separately identified mouse and human pairs of coding/non-coding RNAs within 100 kb from each other. Next, we identified conserved human/mouse genes with a neighbouring non-coding RNA. After this selection process, we obtained 7348 mouse coding genes, 2698 mouse non-coding RNAs, 7770 human coding genes, and 6195 human non-coding RNAs. We used the R package limma68 to identify differential RNAs for each of the comparisons AT1 over MCF10A, DCIS over AT1, and CA1D over DCIS. We analysed separately the selected 7770 human coding genes, and the 6195 non-coding RNAs, using the fold change cut-off of 1.5× and the FDR-adjusted p-value cut-off of 0.1. We applied the additional constraints that coding genes and non-coding RNAs should be within 100 kb of each other when comparing the same pairs of cells. Finally, we obtained 882 coding and 540 non-coding RNAs. We next considered the corresponding 890 mouse coding genes, and their neighbouring 591 non-coding RNAs. We used limma68 to analyse RNAs differentially expressed between WT and TAp63−/− MECs. Using the cut-off p-value<0.05 and fold change exceeding 1.5×, and the genomic distance of at most 100 kb between a coding gene and neighbouring non-coding RNA, we obtained 11 coding genes and 12 non-coding RNAs. Mouse non-coding RNAs were further validated via RT-qPCR, and those that passed, as well as their human counterparts, were depicted graphically as heatmaps using the Python SciPy scientific library.

(3) Quantitative Real Time PCR.

Total RNA was prepared using TRIzol reagent (Invitrogen)5. For gene expression analysis, complementary DNA was synthesized from 5 μg of total RNA using the SuperScript II First-Strand Synthesis Kit (Invitrogen) according to the manufacturer's protocol followed by qRT-PCR using the TaqMan® Universal PCR Master Mix (Applied Biosystems). qRT-PCR was performed using a QuantStudio 6 flex PCR machine (Applied Biosystems) and each qRT-PCR was performed in triplicate. The utilized primers are listed in Table 1.

TABLE 1

| Species | Gene Name | TaqMan Assay ID |
|---|---|---|
| Mouse | TROLL-1_{(RP23-168E14.4)} | Mm01219093_m1 |
| Mouse | TROLL-2_{(1700006J14Rik)} | Mm01164814_m1 |
| Mouse | TROLL-3_{(E130307A14Rik)} | Mm04237550_m1 |
| Mouse | TROLL-4_{(2610307P16Rik)} | Mm01129970_m1 |
| Mouse | TROLL-5_{(AC122821.1)} | Mm00467926_m1 |
| Mouse | TROLL-6_{(2410006H16Rik)} | Mm01331817_g1 |
| Mouse | TROLL-7_{(Malat1)} | Mm01227912_s1 |
| Mouse | TROLL-8_{(AC157572.1)} | Mm01731578_gH |
| Mouse | TROLL-9_{(4930481B07}) | Mm01257798_g1 |
| Mouse | GAPDH | Mm99999915_g1 |
| Human | TROLL-1_{(RP11-98G7.1)} | Hs00418864_m1 |
| Human | TROLL-2_{(RPSAP52)} | Hs03677485_m1 |
| Human | TROLL-3_{(TRAF3IP2-AS1)} | Hs04274045_m1 |
| Human | TROLL-4_{(NR}_{_{—}}₀₁₅₄₁₀₎ | Hs01371948_m1 |
| Human | TROLL-5_{(LINC00514)} | Hs04273767_g1 |
| Human | TROLL-6_{(RP11-138I1.3)} | Hs00415106_m1 |
| Human | TROLL-7_{(MALAT1)} | Hs00273907_s1 |
| Human | TROLL-8_{(AL161668.12)} | Hs00922963_s1 |
| Human | TROLL-9_{(RP11-126K1.6)} | Hs04937869_g1 |
| Human | TAp63 | Hs00186613_m1 |
| Human | IFIT1 | Hs03027069_s1 |
| Human | ITG3BP | Hs00183924_m1 |
| Human | KCTD7 | Hs00399233_m1 |
| Human | MAD2L2 | Hs01057448_m1 |
| Human | NCOA5 | Hs01001913_m1 |
| Human | TERB2 | Hs01014975_m1 |
| Human | WDR26 | Hs01553459_m1 |
| Human | POLR2A | Hs00172187_m1 |

(4) Plasmid and siRNA Transfection.

pBabe-RPSAP52 (TROLL-2) was generated by subcloning RPSAP52 from pBluescript II SK hRPSAP52 (BC107865, Dharmancon) into pBabe-hygro (#1765, Addgene). pBluescript II SK TROLL-2 Δ522-538 was generated via deletion of the indicated nucleotides from pBluescript II SK hRPSAP52. pBabe-TRAF3IP2-AS1 (TROLL-3) was generated by subcloning TRAF3IP2-AS1 from pCMV-SPORT6 hTRAF3IP2-AS1 (BC043575, Dharmacon) into pBabe-hygro (#1765, Addgene). pCMV-SPORT6 TROLL-3 Δ467-482 was generated via deletion of the indicated nucleotides from pCMV-SPORT6 hTRAF3IP2-AS1. For DNA transfection Lipofectamine 2000 (Invitrogen) was used and for siRNA transfection double-stranded RNA oligos (40 nM) were transfected using Lipofectamine RNAiMax (Invitrogen) according to the manufacturer's instructions. The universal negative siRNA control #1 (siControl) was purchased from Sigma (SIC001-10NMOL). Additional siRNAs utilized were: siIFIT1 (SASI_Hs01_00017406, Sigma), siITG3BP (SASI_Hs01_00238825, Sigma), siKCTD7 (SASI_Hs01_00228145, Sigma), siMAD2L2 (SASI_Hs02_00329127, Sigma), siNCOAS (SASI_Hs01_00172441, Sigma), siTERB2 (SASI_Hs01_00102225, Sigma), siNOLC1 (SASI_Hs01_00116300, Sigma), siWDR26 (SASI_Hs01_00029068, Sigma), and siWDR26 3′UTR (5′-UGAUAGAAAGAGUGCAUUA-3′)(SEQ ID NO: 142). The sequences of the siRNA pools used to target the lncRNAs are listed in Table 2.

TABLE 2

| Species | Gene Name | Sequence (Sense, 5′-3′) |
|---|---|---|
| Human | TROLL-1_{(RP11-98G7.1)} | GUAACUGAAAGUUACUUAA (SEQ ID NO: 10) |
| | | UCAAUAAUCUCCAUGUUAG (SEQ ID NO: 11) |
| | | CUCACAAGAAGGCUUCAAU (SEQ ID NO: 12) |
| Human | TROLL-2_{(RPSAP52)} | CACUGUUACUCAGCCUGAA (SEQ ID NO: 13) |
| | | GAAGGAAUUUCAGGGUAAA (SEQ ID NO: 14) |
| | | CCUGGAACCUUCCUAACCA (SEQ ID NO: 15) |
| Human | TROLL-3_{(TRAF3IP2-AS1)} | GAAACAACUUCCACUUCCA (SEQ ID NO: 16) |
| | | GGAAUUGAUAGCCUAUAAA (SEQ ID NO: 17) |
| | | GAGCAUUUCCAAUGGAUUA (SEQ ID NO: 18) |
| Human | TROLL-4_{(NR}_015410) | GAAGUACCCUCAGGUGACU (SEQ ID NO: 19) |
| | | CAUCUAGAAAGCAUCAUGA (SEQ ID NO: 20) |
| | | GACUUGAGGGUCAACUUUA (SEQ ID NO: 21) |
| Human | TROLL-5_{(LINC00514)} | CACACUCUGGGUCCCAACA (SEQ ID NO: 22) |
| | | CCUCUACCCUCCUGUGACA (SEQ ID NO: 23) |
| | | CUGUGUCUGCCCUCAAACA (SEQ ID NO: 24) |
| Human | TROLL-6_{(RP11-138I1.3)} | CAUAUCAAUUUAAACUUCA (SEQ ID NO: 25) |
| | | GGACUACAAAUCUUAAUUA (SEQ ID NO: 26) |
| | | GUUGUAAUCUCCAUUUCAG (SEQ ID NO: 27) |
| Human | TROLL-7_{(MALAT1)} | GAAUUCCGGUGAUGCGAGU (SEQ ID NO: 28) |
| | | CUAACGAUUUGGUGGUGAA (SEQ ID NO: 29) |
| | | GUAAAGCCCUGAACUAUCA (SEQ ID NO: 30) |
| Human | TROLL-8_{(AL161668.12)} | CAUCCAUAAAGAAGGCAUA (SEQ ID NO: 31) |
| | | CCACUUAUUGGCCCUCAUU (SEQ ID NO: 32) |
| | | GACUUGUUCUGUCGCUUCU (SEQ ID NO: 33) |
| Human | TROLL-9_{(RP11-126K1.6)} | AGUUUAGUCUACGGUCCUC (SEQ ID NO: 34) |
| | | UGGAUGAUAAAAUAACUAG (SEQ ID NO: 35) |
| | | CUUUUUCUGCUCUCCUCCC (SEQ ID NO: 36) |

(5) Chromatin Immunoprecipitation.

CA1D cells were either treated for 6 days with doxycycline (1 μg/mL) or left untreated. Cellular proteins were crosslinked to DNA using 1% formaldehyde and chromatin was prepared as described earlier10. Each ChIP was performed in triplicate using either a TAp63 specific antibody (sc-8608, Santa Cruz) or IgG purified from mouse serum (sc-2025, Santa Cruz) and rabbit serum (sc-2027, Santa Cruz) as negative control for the immunoprecipitation. The recruitment of TAp63 was analysed by qRT-PCR as previously reported with the primers listed in Table 3.

TABLE 3

| Promoter | Binding Site | Primers (5′-3′) |
|---|---|---|
| TROLL-1_{(RP11-98G7.1)} | TAp63 BS (−2238 to | Forward: CAGAGTGCACTTTCACATGC |
| | −2213) | (SEQ ID NO: 36) |
| | | Reverse: TGCACTGCCTTGTCCATGTT |
| | | SEQ ID NO: 37) |
| | non-specific (−914 to | Forward: GTAGTGACACAGGCGAGGAC |
| | −936) | (SEQ ID NO: 38) |
| | | Reverse: CACGTGCCCCATAAAAGCTG |
| | | (SEQ ID NO: 39) |
| TROLL-2_{(RPSAP52)} | TAp63 BS (−4269 to | Forward: |
| | −4240) | TGCAAGGGTTCAAAGAGGAAACT |
| | | (SEQ ID NO: 40) |
| | | Reverse: |
| | | ATGAGTTGCCAAATGCATAGCC (SEQ |
| | | ID NO: 41) |
| | non-specific (−297 to | Forward: |
| | −273) | TGAAAAGCAAACCACATTTCGAG(SEQ |
| | | ID NO: 42) |
| | | Reverse: |
| | | ACAGATCTAGGAAGCAAAGTGC (SEQ |
| | | ID NO: 43) |
| TROLL-3_{(TRAF3IP2-AS1)} | TAp63 BS (−3825 to | Forward: TGTCGCCCAGGCTGGTCTCC |
| | −3800) | (SEQ ID NO: 44) |
| | | Reverse: TCTTAAGTTGGTGAAATTTG |
| | | (SEQ ID NO: 45) |
| | non-specific (−741 to | Forward: GGGCTGGCCATGTAGTAGTC |
| | −716) | (SEQ ID NO: 46) |
| | | Reverse: CTCCTCGAGGTGCCTCTGT |
| | | (SEQ ID NO: 47) |
| TROLL-4_{(NR}_015410) | TAp63 BS (−4919 to | Forward: ACTCAGTTTGGTATCCAGGG |
| | −4898) | (SEQ ID NO: 48) |
| | | Reverse: TGTTTGTCTCTTCCAGGTGG |
| | | (SEQ ID NO: 49) |
| | non-specific (−1101 to | Forward: TGCTATGGCCCGAAGTTGGG |
| | −1079) | (SEQ ID NO: 50) |
| | | Reverse: CAGGAACAGCTCCCGACC |
| | | (SEQ ID NO: 51) |
| TROLL-5_{(LINC00514)} | TAp63 BS (−2320 to | Forward: CTGTGTGAATCCAGCACCC |
| | −2297) | (SEQ ID NO: 52) |
| | | Reverse: AGCTGGGACGGTCCATTC |
| | | (SEQ ID NO: 53) |
| | non-specific (−428 to | Forward: GAGAATTCAGACAGTGAGG |
| | −406) | (SEQ ID NO: 54) |
| | | Reverse: TGCGGAAGCAAGACAGTGC |
| | | (SEQ ID NO: 55) |
| TROLL-6_{(RP11-138I1.3)} | TAp63 BS (−2287 to | Forward: GCTGGCCTCCAGGATCGC |
| | −2264) | (SEQ ID NO: 56) |
| | | Reverse: CCTGTAAACAGATGGCAAGC |
| | | (SEQ ID NO: 57) |
| | non-specific (−683 to | Forward: AGGTCTTATGATTGTCTTCCC |
| | −658) | (SEQ ID NO: 58) |
| | | Reverse: CCTTTCAGCCTATGAACATGC |
| | | (SEQ ID NO: 59) |
| TROLL-7_{(MALAT1)} | TAp63 BS (−3874 to | Forward: GCACTCTAGCCTGGGTGAC |
| | −3851) | (SEQ ID NO: 60) |
| | | Reverse: TCTTCATGGCACCTGGATTCC |
| | | (SEQ ID NO: 61) |
| | non-specific (-695 to | Forward: ACAGCGTCACTAATCTCTCC |
| | −674) | (SEQ ID NO: 62) |
| | | Reverse: |
| | | CCTAAAATGGAGAGTTCCAGG (SEQ |
| | | ID NO: 63) |
| TROLL-8_{(AL161668.12)} | TAp63 BS (−2034 to | Forward: CTGCCTGATCTCCATGTAGC |
| | −2013) | (SEQ ID NO: 64) |
| | | Reverse: |
| | | CACTGTAGTTGAGAAAATGAGC (SEQ |
| | | ID NO: 65) |
| | non-specific (−3379 to | Forward: |
| | −3359) | TGAAGAGAATGAGAAAATGTGG (SEQ |
| | | ID NO: 66) |
| | | Reverse: |
| | | CAAAGTGCTAGGATTACAGGC (SEQ |
| | | ID NO: 67) |
| TROLL-9_{(RP11-126K1.6)} | TAp63 BS (−3278 to | Forward: CTGAAAGTGGCTCAGGGTTG |
| | −3254) | (SEQ ID NO: 68) |
| | | Reverse: GAGCGGATCCTGAAACGGT |
| | | (SEQ ID NO: 69) |
| | non-specific (−330 to | Forward: TCCATTCTATCTGCCCCACCT |
| | −309) | (SEQ ID NO: 70) |
| | | Reverse: |
| | | TTGTTTTTGAGGGGGAGGAGG (SEQ |
| | | ID NO: 71) |

(6) Cell Proliferation and Apoptosis Assays.

Cells were plated at a density of 1×10⁴ cells in 6 replicates in a 96-well plate. Three biological replicates were performed per each assay. To evaluate cell proliferation, the cells were labelled for 3 h with 10 mM EdU (5′-ethynyl-2′-deoxyuridine) and stained using the Click-iT EdU microplate assay (Invitrogen). Apoptosis was monitored by incubating the cells with Annexin V-Alexa Fluor 488 (Essen BioScience) according to the manufacturer's instructions Images were captured and percent of either cell proliferation or apoptosis was quantified using a high-throughput immunofluorescence plate reader and accompanying software (IncuCyte, Essen Bioscience).

(7) Cell Migration and Invasion Assays.

1×10⁴ cells in DMEM/F12 (1:1) media containing 0.5% horse serum were plated in 6 replicates in an IncuCyte ClearView 96 well cell migration plate (Essen BioScience), whose wells were either left uncoated (cell migration) or coated with 20 μL of 200 μg/mL growth factor reduced matrigel (Corning) (cell invasion). As chemo-attractant, DMEM/F12 (1:1) media containing 5% horse serum, 10 μg/mL insulin, 20 ng/mL epidermal growth factor, and 500 ng/mL hydrocortisone was used in the bottom chambers. Images were captured and percent of either cell migration or invasion was quantified using a high-throughput plate reader and accompanying software (IncuCyte, Essen Bioscience).

(8) Orthotopic Xenograft Mouse Model.

Female athymic nu/nu mice (6 weeks old) were used for all the experiments and randomized into three groups of 5 mice each: i) cells infected with shRNA control (shNT); ii) cells infected with shRNA for TROLL-2 (shTROLL-2); and cells infected with shRNA for TROLL-3 (shTROLL-3). For the experiments involving the breast cancer cell lines, 2×10⁶ cells (CA1D) or 2.5×10⁶ cells (MDA MB-231) in 100 μL of growth factor reduced matrigel (Corning) were implanted orthotopically into the 4th pair of mammary fat pads. For the experiments involving the lung cancer cell lines, 1×10⁶ cells (H1299) or 2×10⁶ cells (H358) in 100 μL of PBS were delivered via intrapulmonary injection as previously reported40. For the experiments involving the melanoma cell lines (A375 and Malme-3M), 1×10⁷ cells in 100 μL of PBS were subcutaneously injected in both flanks. Mice were fed with doxycycline containing food (200 mg/kg) to induce the expression of the shRNA and target the lncRNA of interest for the entire duration of the experiment, which was either 5 weeks (MDA MB-231), 6 weeks (H1299, A375, and Malme-3M), 8 weeks (H358), or 10 weeks (CA1D). At the indicated end point, the tumor xenografts were collected, measured with a calliper, and analysed using ISH. For the experiments involving DCIS cells, female athymic nu/nu mice (6 weeks old) were randomized into two groups of 5 mice each: DCIS infected with pBabe Empty and DCIS infected with both pBabe TROLL-2 and pBabe TROLL-3. The obtained tumor xenografts were collected 5 weeks after the injection, measured with a calliper, and analysed using IHC. All procedures were approved by the IACUC at the H. Lee Moffitt Cancer Center and Research Institute.

(9) Tail Vein Injections.

Female athymic nu/nu mice (6 weeks old) were randomized into three groups of 5 mice each as described above for the orthotopic injections. The following amounts of cells in 100 μL of PBS were injected in the tail vein of the mice: 5×10⁵ cells (CA1D), 1×10⁶ cells (MDA MB-231), 5×10⁶ cells (A375 and Malme-3M). Mice were fed with doxycycline containing food (200 mg/kg) to induce the expression of the shRNA and target the lncRNA of interest throughout the duration of the experiment, which was either 4 weeks (MDA MB-231), 8 weeks (A375 and Malme-3M), or 10 weeks (CA1D). At the indicated end point, the lungs were collected and fixed in buffered formalin. Hematoxylin and eosin (H&E) stained cross sections were then used to quantify the area of the lungs colonized by the cancer cells via the Oncotopix® software (Visiopharm). All procedures were approved by the IACUC at the H. Lee Moffitt Cancer Center and Research Institute.

(10) Intracardiac Injections.

Female athymic nu/nu mice (6 weeks old) were randomized 646 into three groups of 5 mice each as described above for the orthotopic injections. 8×10⁵ 647 cells (H1299) and 2×10⁶ cells (H1299) in 100 μL of PBS were delivered via intracardiac 648 injection as previously described41. Mice were fed with doxycycline containing food (200 mg/kg) to induce the expression of the shRNA and target the lncRNA of interest. 4 weeks after the injection, the lungs were collected and fixed in buffered formalin. Hematoxylin and eosin (H&E) stained cross sections were then used to quantify the area of the lungs colonized by the cancer cells via the ONCOTOPIX® software (Visiopharm). All procedures were approved by the IACUC at the H. Lee Moffitt Cancer Center and Research Institute.

(11) Moffitt Tissue Microarrays.

The Moffitt tissue microarrays (TMAs) used in this study consisted of: i) 68 breast cancer samples comprising 43 triple negative and 25 non-triple negative breast cancers (TMA-5)29; and ii) 100 melanoma samples and 6 control cases (TMA-4). The formalin-fixed and paraffin-embedded biopsies were used to produce 0.6 mm cores, which were assembled into the two TMAs by the Tissue Core Facility at the H. Lee Moffitt Cancer Center & Research Institute under delegated ethical authority of the Moffitt Research Ethics Committee with written informed consent from contributing patients.

(12) In Situ Hybridization of Xenograft Tumors and Tissue Microarrays.

The xenograft tumors, tissue microarrays (TMAs) of breast cancer progression (BR480a, US Biomax), colon cancer progression (C0961, US Biomax), lung cancer progression (BC04002a, US Biomax), ovarian cancer progression (OV1005b, US Biomax), two TMAs of melanoma progression (ME1004f, US Biomax; and the Moffitt TMA-4, Moffitt Cancer Center), and three TMAs of invasive breast cancers (BR20837a, US Biomax; the Dundee TMA27, Tayside Tissue Bank; and the Moffitt TMA-5, Moffitt Cancer Center) were used for the in situ hybridization (ISH) assay. The double digoxigenin labelled LNA probes (Exiqon) utilized for ISH were: _TROLL-2 (5′-ACAGAAGCTTGCAGGGAACCT-3′) (SEQ ID NO: 72); _TROLL-3 (5′-ACTATTACTGCTAACTAACTTATGGA-3′) (SEQ ID NO: 73).

As a negative control, the double digoxigenin labelled scramble LNA probe (339508, Exiqon) was used. The ISH was performed using the Exiqon protocol for FFPE tissue, and the hybridization step was done using a 150 nM final concentration of the LNA probes at 55° C. for 1 hour in the Dako hybridizer (Agilent). The LNA probes were then detected with Alkaline Phosphatase (AP) conjugated antibody (11093274910, Sigma, 1:400), and visualized via a chromogenic reaction converting the AP substrate NBT-BCIP (11697471001, Roche) into an alcohol insoluble purple precipitate. Nuclear Fast Red™ (H-3403, Vector laboratories) was used as a counterstain. The signal intensity (continuous variable, 0 to 1) and the proportion of positive tissue (continuous variable, 0% to 100%) were measured using the Oncotopix® software (Visiopharm). The ISH score was then quantified by multiplying the signal intensity by the proportion of positive tissue, giving a value comprised between 0 and 100, and visualized using the Circos software.

(13) Immunohistochemistry of Xenograft Tumors and Tissue Microarrays.

CA1D-derived xenograft tumors, tissue microarrays (TMAs) of breast cancer progression (BR480a, US Biomax), colon cancer progression (C0961, US Biomax), lung cancer progression (BC04002a, US Biomax), ovarian cancer progression (OV1005b, US Biomax), two TMAs of melanoma progression (ME1004f, US Biomax; and the Moffitt TMA-4, Moffitt Cancer Center), and three TMAs of invasive breast cancers (BR20837a, US Biomax; the Dundee TMA, Tayside Tissue Bank; and the Moffitt TMA-5, Moffitt Cancer Center) were used for immunohistochemistry (IHC). IHC was performed as previously described, and the following primary antibodies were used: NCOA5 (ab70831, Abcam, 1:200), WDR26 (ab203345, Abcam, 1:200), TAp63 (618902, Biolegend, 1:200) and pAKT (S473) (4060S, Cell Signaling, 1:100). In the case of the TMAs, the signal intensity (continuous variable, 0 to 1) and the proportion of positive tissue (continuous variable, 0% to 100%) were measured using the ONCOTOPIX® software (Visiopharm). The IHC score was then quantified by multiplying the signal intensity by the proportion of positive tissue, giving a value comprised between 0 and 100, and visualized using the Circos software.

(14) Cy5 Labelling of lncRNAs and Protein Microarray Analysis.

In vitro transcription of the sense and antisense strands of RPSAP52 (TROLL-2), TRAF3IP2-AS1 (TROLL-2), and their respective deletion mutants (TROLL-2 Δ522-538 and TROLL-3 D467-482) was performed from pBluescript II SK and pCMV-SPORT6, respectively. Following transcription, the strands were labelled with Cy5 using the Label IT μArray Cy5 labelling kit (Mirus) with a labelling efficiency of 3 pmol Cy5 dye per μg of RNA. Protoarray Human Protein Microarrays v5.0 (ThermoFisher Scientific) were used for the hybridization with 10 pmol of either Cy5 labelled sense or antisense as a negative control. 3 independent replicates were carried out as previously described34, with the hybridization step performed at 4° C. for 1 hr in the dark. The slides were then scanned with the GenePix 4000B Microarray (Molecular Devices) at 635 nm for the selection of proteins interacting exclusively with the sense strands.

(15) In Vitro RNA Pull-Down Coupled with Protein Detection.

For the in vitro RNA pull-down, the magnetic RNA-protein pull-down kit (Pierce) was used according to the manufacturer's instructions. Briefly, in vitro transcribed lncRNAs were end-labelled with desthiobiotin. 50 pmol of labelled lncRNA was incubated with 50 μl streptavidin magnetic beads for 30 min at 25° C. with agitation. Streptavidin magnetic bead-bound lncRNA was then incubated with cell lysate (33-330 μg) of either CA1D cells (for endogenous WDR26), CA1D cells overexpressing FLAG-tagged WDR26 (OHu01176D, GenScript), or HEK293T cells overexpressing FLAG-tagged NCOA5 (OHu03595D, GenScript). After an overnight incubation at 4° C. with gentle end-to-end rotation, the beads were washed three times with 1× Wash Buffer provided in the kit. After the final wash, streptavidin magnetic beads were resuspended in 50 μl of Elution Buffer provided in the kit, and the eluted RNA-bound proteins were analysed by SDS-PAGE as previously reported6l. The detection of FLAG-tagged WDR26 and FLAG-tagged NCOA5 was performed with the anti-Flag antibody (A8592, Sigma, 1:1000). The detection of endogenous WDR26 was performed with the anti-WDR26 antibody (ab85961, Abcam, 1:2000).

(16) Nuclear and Cytoplasmic Protein Fractionation.

3×10⁶ cells were used for the extraction of nuclear and cytoplasmic proteins with the subcellular protein fractionation kit for cultured cells (78840, ThermoFisher Scientific) in accordance to the manufacturer's instructions. 10 μg of the nuclear and cytoplasmic fractions were then analysed by SDS-PAGE as previously reported, and the following primary antibodies were used: WDR26 (ab85961, Abcam, 1:2000), NOLC1 (sc-374033, Santa Cruz, 1:1000), FLAG (A8592, Sigma, 1:1000), H3 (ab1791, Abcam, 1:2000), and HSP90 (ab13495, Abcam, 1:5000).

(17) Kaplan-Meier Curves of TCGA Data.

To assess the clinical significance of composite protein coding genes (PCG) and lncRNAs we first downloaded the TCGA breast cancer and melanoma data transcriptome profiles using the Firebrowse portal. For a combination of PCG and lncRNA, the samples were first separated into two equal bins with respect to each RNA expression. Resulting groupings from PCG and lncRNA were combined so each sample will belong to one of the four groups. The 4 groups were: 1) PCG expression>median, lncRNA expression>median; 2) PCG expression>median, lncRNA expression<median; 3) PCG expression<median, lncRNA expression>median; and 4) PCG expression<median, lncRNA expression<median. Groups association with survival was assessed using the survival package71 in the R statistical system.

(18) WDR26 Deletion Mutants.

The amino acid sequence of WDR26 was analysed for the presence of a nuclear localization signal (NLS) with cNLS Mapper72, and of a nuclear export signal (NES) with NetNES73. The identified NLS was between aa 111 and aa 121 (GSSLKKKKRLS) (SEQ ID NO: 74), while the NES was localized between aa 224 and aa 236 (LEDGKVLEEALQVL) (SEQ ID NO: 75). These sequences were deleted from pcDNA3.1-WDR26 FLAG (OHu01176D, GenScript) to produce WDR26-ANLS and WDR26-ANES, respectively.

(19) Liquid Chromatography-Mass Spectrometry (LC-MS/MS) Analysis.

1×10⁶ CA1D were transfected with either WDR26 FLAG, WDR26-ANLS FLAG, WDR26-ANES, or pcDNA3.1-FLAG as a negative control. 24 h after the transfection, the cells were lysed and the IP assay was performed as previously reported27 using 25 μl of the anti-FLAG M2 magnetic beads (M8823, Sigma). The samples were processed as described previously and the identified peptides are listed in Supplementary table 6.

(20) Co-immunoprecipitation (CoIP) assay.

1×10⁷ CA1D were transfected with siRNAs for TROLL-2, TROLL-3, or with the non-targeting siRNA as a negative control. To test the interaction between WDR26 and AKT, 24 h after the transfection the cells were serum-starved for 24 h and subsequently treated for 10 min with 10 μM lysophosphatidic acid (LPA). To test the interaction between WDR26 and NOLC1, the cells were kept in their growth media for 48 h. The cells were then lysed and the CoIP assay was performed as previously reported27, and 1 μg of each of following primary antibodies was utilized per sample: AKT (9272S, Cell Signaling), NOLC1 (ab184550, Abcam), WDR26 (ab203345, Abcam), and normal rabbit IgG (sc-2027, Santa Cruz) as negative control. The interaction was then detected via western blot using the following primary antibodies: pAKT (S473) (4060S, Cell Signaling, 1:100), AKT (ab8805, Abcam, 1:1000), and WDR26 (ab85961, Abcam, 1:2000).

(21) LPA treatment.

1×10⁶ CA1D cells were transfected either with the siControl or with the siWDR26 3′UTR in combination with either pcDNA3.1-FLAG, pcDNA3.1-WDR26 FLAG, pcDNA3.1-WDR26-ΔNLS FLAG, or pcDNA3.1-WDR26-ΔNES FLAG. 24 h after the transfection, the cells were serum-starved for 24 h and subsequently treated for 10 min with 10 lysophosphatidic acid (LPA). The cells were then lysed and western blot was performed as previously reported6l, and the following primary antibodies were used: pAKT (S473) (4060S, Cell Signaling, 1:100), AKT (ab8805, Abcam, 1:1000), WDR26 (ab85961, Abcam, 1:2000), FLAG (A8592, Sigma, 1:1000), and Actin (A5441, Sigma, 1:5000).

(22) Cross-Linking Immunoprecipitation and qRT-PCR (CLIP-qPCR) Assay.

To test the interaction between the lncRNAs and either AKT or WDR26, 1×10⁷ CA1D were transfected with siRNAs for WDR26 or with the non-targeting siRNA as a negative control. 48 h after the transfection, the CLIP assay was performed without an RNA treatment step as previously reported50, 51 and using 1 μg of each of the following primary antibodies: AKT (9272S, Cell Signaling), WDR26 (ab203345, Abcam), and normal rabbit IgG (sc-2027, Santa Cruz) as negative control. The presence of the lncRNAs in the CLIP-ed samples was assessed via qRT-PCR using the Taqman probes listed in Table 1. To map the regions of TROLL-2 and TROLL-3 interacting with WDR26, 1×10⁷ CA1D were utilized. The CLIP assay was performed including an RNA treatment step as previously reported50, 51 and using 1 μg of each of the following primary antibodies: WDR26 (ab203345, Abcam) and normal rabbit IgG (sc-2027, Santa Cruz) as negative control. The presence of the lncRNA fragments in the CLIP-ed samples was assessed via qRT-PCR using the primers listed in Table 5.

TABLE 5

| Name | Primers (5′-3′) |
|---|---|
| TROLL-2 segment 1 (5 to | Forward: CCCATTTAGAGAATTCGGGAAG (SEQ ID |
| 127) | NO: 76) |
| | Reverse: AGTTAAGGCAGAGTTCACAAAG (SEQ ID |
| | NO: 77) |
| TROLL-2 segment 2 (116 to | Forward: TCTGCCTTAACTCAAAGATTCC (SEQ ID |
| 208) | NO: 78) |
| | Reverse: CATCCTCCTCCTTCATTTGC (SEQ ID NO: |
| | 79) |
| TROLL-2 segment 3 (189 to | Forward: GCAAATGAAGGAGGAGGATG (SEQ ID NO: |
| 317) | 80) |
| | Reverse: TCAGATTTATGCTGTAGATGCC (SEQ ID |
| | NO: 81) |
| TROLL-2 segment 4 (303 to | Forward: ACAGCATAAATCTGAAGAGGAC (SEQ ID |
| 403) | NO: 82) |
| | Reverse: CCTGGAGGATATGACACTGAC (SEQ ID |
| | NO: 83) |
| TROLL-2 segment 5 (382 to | Forward: TGTCAGTGTCATATCCTCCAG (SEQ ID NO: |
| 540) | 84) |
| | Reverse: GTCAGAAACCACAAGAAGCC (SEQ ID NO: |
| | 85) |
| TROLL-2 segment 6 (489 to | Forward: TAACCAGATCCAGGCAGCCTAC (SEQ ID |
| 636) | NO: 86) |
| | Reverse: TGTCCAATGGCACAGAGGAG (SEQ ID NO: |
| | 87) |
| TROLL-2 segment 7 (627 to | Forward: CCATTGGACATTGCCATCAC (SEQ ID NO: |
| 738) | 88) |
| | Reverse: TGTTGCAGGAAATGGTGC (SEQ ID NO: 89) |
| TROLL-2 segment 8 (722 to | Forward: CACCATTTCCTGCAACACC (SEQ ID NO: |
| 864) | 90) |
| | Reverse: GCTGAAACAGTCCATTTACCC (SEQ ID NO: |
| | 91) |
| TROLL-2 segment 9 (807 to | Forward: CTGCTGCTGAAAAGGCTGTGAC (SEQ ID |
| 944) | NO: 92) |
| | Reverse: ACTGCTGAATAGGCACAGAGGG (SEQ ID |
| | NO: 93) |
| TROLL-2 segment 10 (929 to | Forward: GTGCCTATTCAGCAGTTTCTAC (SEQ ID |
| 1058) | NO: 94) |
| | Reverse: TGCAAGAACAGCTTAAGACC (SEQ ID NO: |
| | 95) |
| TROLL-2 segment 11 (1038 to | Forward: TGGTCTTAAGCTGTIVTTGCAC (SEQ ID |
| 1108) | NO: 96) |
| | Reverse: ACTGATCITITATITICTGTCAACCT (SEQ |
| | ID NO: 97) |
| TROLL-3 segment 1 (7 to | Forward: GCTTGCGGGAGGGGGGC (SEQ ID NO: 98) |
| 117) | Reverse: CTGCTGGATGTGAAATGGCG (SEQ ID NO: |
| | 99) |
| TROLL-3 segment 2 (96 to | Forward: ACCGCCATTTCACATCCAG (SEQ ID NO: |
| 195) | 100) |
| | Reverse: TGGGCAATATAGTGAGACCTC (SEQ ID |
| | NO: 101) |
| TROLL-3 segment 3 (187 to | Forward: TATTGCCCAAGCTGGTCTC (SEQ ID NO: |
| 292) | 102) |
| | Reverse: ATAATTTCGCAGGGCATGG (SEQ ID NO: |
| | 103) |
| TROLL-3 segment 4 (255 to | Forward: AGATAACAGGTATGAGCCACC (SEQ ID |
| 360) | NO: 104) |
| | Reverse: GCAGTGAGTTGAGATTGGG (SEQ ID NO: |
| | 105) |
| TROLL-3 segment 5 (342 to | Forward: CCCAATCTCAACTCACTGC (SEQ ID NO: |
| 489) | 106) |
| | Reverse: GGGCAATATAGTGAAACCCTG (SEQ ID |
| | NO: 107) |
| TROLL-3 segment 6 (472 to | Forward: GGTTTCACTATATTGCCCAGG (SEQ ID NO: |
| 621) | 108) |
| | Reverse: AAGGAAAGAAAAGGGATGACAC (SEQ ID |
| | NO: 109) |
| TROLL-3 segment 7 (603 to | Forward: TCATCCCTTTTCTTTCCTTCAC (SEQ ID NO: |
| 749) | 110) |
| | Reverse: AATGCCTCCAGATATTTTGCC (SEQ ID NO: |
| | 111) |
| TROLL-3 segment 8 (727 to | Forward: TTGGCAAAATATCTGGAGGC (SEQ ID NO: |
| 837) | 112) |
| | Reverse: TTCAACACCTGCACTATTGAC (SEQ ID NO: |
| | 113) |
| TROLL-3 segment 9 (805 to | Forward: CAGGTCCAAAGTGTCAATAGTG (SEQ ID |
| 943) | NO: 114) |
| | Reverse: AAATGCCTCTGAAAGGGAATG (SEQ ID |
| | NO: 115) |
| TROLL-3 segment 10 (884 to | Forward: AGTCTCTTGCCTTTATGAAACC (SEQ ID |
| 1029) | NO: 116) |
| | Reverse: GCAGAAACCTGCATATAAAGCC (SEQ ID |
| | NO: 117) |
| TROLL-3 segment 11 (972 to | Forward: TGGAGTCAATTGCATCAACC (SEQ ID NO: |
| 1071) | 118) |
| | Reverse: TATGTGCCTACCTTGTACCC (SEQ ID NO: |
| | 119) |
| TROLL-3 segment 12 (1051 to | Forward: TGGGTACAGGTAGGCACAT (SEQ ID NO: |
| 1185) | 120) |
| | Reverse: GGAAATTCATCTCTCACCACATGA (SEQ ID |
| | NO: 121) |
| TROLL-3 segment 13 (1168 to | Forward: GGTGAGAGATGAATTTCCTGAACA (SEQ |
| 1284) | ID NO: 122) |
| | Reverse: TGC7TAACITATGGAACITGGAAGTT (SEQ |
| | ID NO: 123) |
| TROLL-3 segment 14 (1261 to | Forward: ACTTLCACTTCCATAAGTTAGCAG (SEQ |
| 1412) | ID NO: 124) |
| | Reverse: AATGAGGGTCCTGGAAAGCTC (SEQ ID |
| | NO: 125) |
| TROLL-3 segment 15 (1391 to | Forward: TGAGCTTTCCAGGACCCTCATT (SEQ ID |
| 1496) | NO: 126) |
| | Reverse: TCTATGTGTGTTTGGTCTCCTCC (SEQ ID |
| | NO: 127) |
| TROLL-3 segment 16 (1475 to | Forward: GAGGAGACCAAACACACATAGAGT (SEQ |
| 1603) | ID NO: 128) |
| | Reverse: TTTGGGGCCAGGGGAAACTC (SEQ ID |
| | NO: 129) |
| TROLL-3 segment 17 (1592 to | Forward: CCMGCCCCAAACITITATTGC (SEQ ID NO: |
| 1666) | 130) |
| | Reverse: AGCTTTTTAGTGGGAGITITTCTGG (SEQ |
| | ID NO: 131) |
| TROLL-3 segment 18 (1637 to | Forward: ACATTCCAGAAAAACTCCCAC (SEQ ID |
| 1747) | NO: 132) |
| | Reverse: GGGCTCAAGCAATAATCTCAC (SEQ ID |
| | NO: 133) |
| TROLL-3 segment 19 (1742 to | Forward: GAGCCCAGAAGTTTGAGACC (SEQ ID NO: |
| 1841) | 134) |
| | Reverse: GGGACTACAGGCATACATCAC (SEQ ID |
| | NO: 135) |
| TROLL-3 segment 20 (1819 to | Forward: TGGTGATGTATGCCTGTAGTC (SEQ ID NO: |
| 1966) | 136) |
| | Reverse: TGTGTGTTTTTTGAGACAGGG (SEQ ID NO: |
| | 137) |
| TROLL-3 segment 21 (1906 to | Forward: ATGATCACCACTGCACTCC (SEQ ID NO: |
| 2045) | 138) |
| | Reverse: CTTTTCTGGCCTCTGGAATG (SEQ ID NO: |
| | 139) |
| TROLL-3 segment 22 (2025 to | Forward: TCATTCCAGAGGCCAGAAAAG (SEQ ID |
| 2174) | NO: 140) |
| | Reverse: TTCTTTATTGTCCTCCATGTTTTCC (SEQ ID |
| | NO: 141) |

### D. References

Adorno M, et al. A Mutant-p53/Smad complex opposes p63 to empower TGFbeta-induced metastasis. Cell 137, 87-98 (2009).
Alt E U, et al. TRAF3IP2, a novel therapeutic target in glioblastoma multiforme. Oncotarget 9, 29772-29788 (2018).
Avram S, et al. Standardization of A375 human melanoma models on chicken embryo chorioallantoic membrane and Balb/c nude mice. Oncol Rep 38, 89-99 (2017).
Bhardwaj A, et al. Regulation of miRNA-29c and its downstream pathways in preneoplastic progression of triple-negative breast cancer. Oncotarget 8, 19645-19660 (2017).
Boac B M, et al. Expression of the BAD pathway is a marker of triple-negative status and poor outcome. Sci Rep 9, 17496 (2019).
Cabili M N, et al. Integrative annotation of human large intergenic noncoding RNAs reveals global properties and specific subclasses. Genes Dev 25, 1915-1927 (2011).
Cancer Genome Atlas N. Comprehensive molecular characterization of human colon and rectal cancer. Nature 487, 330-337 (2012).
Cancer Genome Atlas N. Comprehensive molecular portraits of human breast tumors. Nature 490, 61-70 (2012).
Cancer Genome Atlas N. Genomic Classification of Cutaneous Melanoma. Cell 161, 1681-1696 (2015).
Cancer Genome Atlas Research N. Comprehensive genomic characterization of squamous cell lung cancers. Nature 489, 519-525 (2012).
Cancer Genome Atlas Research N. Comprehensive molecular profiling of lung adenocarcinoma. Nature 511, 543-550 (2014).
Cancer Genome Atlas Research N. Integrated genomic analyses of ovarian carcinoma. Nature 474, 609-615 (2011).
Chen Y, Peng Y, Fan S, Li Y, Xiao Z X, Li C. A double dealing tale of p63: an oncogene or a tumor suppressor. Cell Mol Life Sci, (2017).
Deng M, Bragelmann J, Kryukov I, Saraiva-Agostinho N, Perner S. FirebrowseR: an R client to the Broad Institute's Firehose Pipeline. Database (Oxford) 2017, (2017).
Foulkes W D, Smith I E, Reis-Filho J S. Triple-negative breast cancer. N Engl J Med 363, 1938-1948 (2010).
Girardini J E, et al. A Pinl/mutant p53 axis promotes aggressiveness in breast cancer. Cancer Cell 20, 79-91 (2011).
Guerrero-Zotano A, Mayer I A, Arteaga C L. PI3K/AKT/mTOR: role in breast cancer progression, drug resistance, and treatment. Cancer Metastasis Rev 35, 515-524 (2016).
Gutschner T, et al. The noncoding RNA MALAT1 is a critical regulator of the metastasis phenotype of lung cancer cells. Cancer Res 73, 1180-1189 (2013).
Harrow J, et al. GENCODE: the reference human genome annotation for The ENCODE Project. Genome Res 22, 1760-1774 (2012).
Hu X, Sood A K, Dang C V, Zhang L. The role of long noncoding RNAs in cancer: the dark matter matters. Curr Opin Genet Dev 48, 8-15 (2017).
Huang N S, et al. Long non-coding RNA metastasis associated in lung adenocarcinoma transcript 1 (MALAT1) interacts with estrogen receptor and predicted poor survival in breast cancer. Oncotarget 7, 37957-37965 (2016).
Janku F, Yap T A, Meric-Bernstam F. Targeting the PI3K pathway in cancer: are we making headway? Nat Rev Clin Oncol 15, 273-291 (2018).
Kandoth C, et al. Mutational landscape and significance across 12 major cancer types. Nature 502, 333-339 (2013).
Kim D, Pertea G, Trapnell C, Pimentel H, Kelley R, Salzberg S L. TopHat2: accurate alignment of transcriptomes in the presence of insertions, deletions and gene fusions. Genome Biol 14, R36 (2013).
Koo H M, VanBrocklin M, McWilliams M J, Leppla S H, Duesbery N S, Vande Woude G F. Apoptosis and melanogenesis in human melanoma cells induced by anthrax lethal factor inactivation of mitogen-activated protein kinase kinase. Proc Natl Acad Sci USA 99, 3052-3057 (2002).
Kosugi S, Hasebe M, Tomita M, Yanagawa H. Systematic identification of cell cycle-dependent yeast nucleocytoplasmic shuttling proteins by prediction of composite motifs. Proc Natl Acad Sci USA 106, 10171-10176 (2009).
Krzywinski M, et al. Circos: an information aesthetic for comparative genomics. Genome Res 19, 1639-1645 (2009).
la Cour T, Kiemer L, Molgaard A, Gupta R, Shiver K, Brunak S. Analysis and prediction of leucine-rich nuclear export signals. Protein Eng Des Sel 17, 527-536 (2004).
Lei R, Xue M, Zhang L, Lin Z. Long noncoding RNA MALAT1-regulated microRNA 506 modulates ovarian cancer growth by targeting iASPP. Onco Targets Ther 10, 35-46 (2017).
Li Y, Xu J, Shao T, Zhang Y, Chen H, Li X. RNA Function Prediction. Methods Mol Biol 1654, 17-28 (2017).
Meier U T, Blobel G. Nopp140 shuttles on tracks between nucleolus and cytoplasm. Cell 70, 127-138 (1992).
Miller F R, et al. Xenograft model of progressive human proliferative breast disease. J Nall Cancer Inst 85, 1725-1732 (1993).
Mudge J M, Harrow J. Creating reference gene annotation for the mouse C57BL6/J genome assembly. Mamm Genome 26, 366-378 (2015).
Muller P A, et al. Mutant p53 drives invasion by promoting integrin recycling. Cell 139, 1327-1341 (2009).
Napoli M, et al. DeltaNp63/DGCR8-Dependent MicroRNAs Mediate Therapeutic Efficacy of HDAC Inhibitors in Cancer. Cancer Cell 29, 874-888 (2016).
Napoli M, Flores E R. The family that eats together stays together: new p53 family transcriptional targets in autophagy. Genes Dev 27, 971-974 (2013).
Napoli M, Flores E R. The p53 family orchestrates the regulation of metabolism: physiological regulation and implications for cancer therapy. Br J Cancer 116, 149-155 (2017).
Onn A, et al. Development of an orthotopic model to study the biology and therapy of primary human lung cancer in nude mice. Clin Cancer Res 9, 5532-5539 (2003).
Rao A, Rajkumar T, Mani S. Perspectives of long non-coding RNAs in cancer. Mol Biol Rep 44, 203-218 (2017).
Salehi S, Taheri M N, Azarpira N, Zare A, Behzad-Behbahani A. State of the art technologies to explore long non-coding RNAs in cancer. J Cell Mol Med 21, 3120-3140 (2017).
Sgarra R, et al. High Mobility Group A (HMGA) proteins: Molecular instigators of breast cancer onset and progression. Biochim Biophys Acta Rev Cancer 1869, 216-229 (2018).
Shalem O, et al. Genome-scale CRISPR-Cas9 knockout screening in human cells. Science 343, 84-87 (2014).
Singla A K, Downey C M, Bebb G D, Jirik F R. Characterization of a murine model of metastatic human non-small cell lung cancer and effect of CXCR4 inhibition on the growth of metastases. Oncoscience 2, 263-271 (2015).
Siprashvili Z, et al. Identification of proteins binding coding and non-coding human RNAs using protein microarrays. BMC Genomics 13, 633 (2012).
Sledge G W, Mamounas E P, Hortobagyi G N, Burstein H J, Goodwin P J, Wolff A C. Past, present, and future challenges in breast cancer treatment. J Clin Oncol 32, 1979-1986 (2014).
Smyth G K. Linear models and empirical bayes methods for assessing differential expression in microarray experiments. Stat Appl Genet Mol Biol 3, Article3 (2004).
Steeg P S. Targeting metastasis. Nat Rev Cancer 16, 201-218 (2016).
Stewart P A, et al. Relative protein quantification and accessible biology in lung tumor proteomes from four LC-MS/MS discovery platforms. Proteomics 17, (2017).
Su X, et al. TAp63 suppresses mammary tumorigenesis through regulation of the Hippo pathway. Oncogene 36, 2377-2393 (2017).
Su X, et al. TAp63 suppresses metastasis through coordinate regulation of Dicer and miRNAs. Nature 467, 986-990 (2010).
Therneau T M G, P. M. Modeling survival data: extending the Cox model. Springer-Verlag New York (2000).
Trapnell C, et al. Differential gene and transcript expression analysis of RNA-seq experiments with TopHat and Cufflinks. Nat Protoc 7, 562-578 (2012).
Ulitsky I, Shkumatava A, Jan C H, Sive H, Bartel D P. Conserved function of lincRNAs in vertebrate embryonic development despite rapid sequence evolution. Cell 147, 1537-1550 (2011).
Ulitsky I. Evolution to the rescue: using comparative genomics to understand long non-coding RNAs. Nat Rev Genet 17, 601-614 (2016).
Venkatanarayan A, et al. LAPP-driven metabolic reprogramming induces regression of p53-deficient tumors in vivo. Nature 517, 626-630 (2015).
Walerych D, Napoli M, Collavin L, Del Sal G. The rebel angel: mutant p53 as the driving oncogene in breast cancer. Carcinogenesis 33, 2007-2017 (2012).
Wang Z, et al. High expression of long non-coding RNA MALAT1 in breast cancer is associated with poor relapse-free survival. Breast Cancer Res Treat 171, 261-271 (2018).
Xu C, Yang M, Tian J, Wang X, Li Z. MALAT-1: a long non-coding RNA and its important 3′ end functional motif in colorectal cancer metastasis. Int J Oncol 39, 169-175 (2011).
Zheng L, et al. Long non-coding RNA MALAT1 regulates BLCAP mRNA expression through binding to miR-339-5p and promotes poor prognosis in breast cancer. Biosci Rep 39, (2019).

1. A method of treating a cancer in a subject comprising knocking down expression of TROLL-1, TROLL-2, TROLL-3, TROLL-5, TROLL-7, and/or TROLL-8 in the subject.

2. The method of treating a cancer in a subject of claim 1, wherein the cancer comprises breast cancer, lung cancer, ovarian cancer, colon cancer, or melanoma.

3-4. (canceled)

5. The method of treating a cancer in a subject of claim 2, wherein the cancer comprises a p53 mutation and/or is Tap63 regulated.

6. (canceled)

7. The method of treating a cancer in a subject of claim 1, wherein expression of TROLL-1, TROLL-2, TROLL-3, TROLL-5, TROLL-7, and/or TROLL-8 is knocked down through the administration of one or more RNA-targeted therapeutics.

8. The method of treating a cancer in a subject of claim 7, wherein the one or more RNA-targeted therapeutics comprises antisense oligonucleotides, siRNA, shRNA, ribozymes, transcription activator-like effector nucleases (TALEN), zinc finger nucleases (ZFNs) and/or clustered regularly interspaced short palindromic repeats/associated (CRISPR/Cas) nucleases.

9. The method of treating a cancer in a subject of claim 7, wherein the expression of TROLL-1 is knocked down by targeting the ENST00000441085.5 and/or ENST00000452465.1 isoform.

10. The method of treating a cancer in a subject of claim 9, wherein the expression of TROLL-1 is knocked down by administering to the subject the siRNA as set forth in SEQ ID NO: 10, SEQ ID NO: 11, and/or SEQ ID NO: 12.

11. The method of treating a cancer in a subject of claim 7, wherein the expression of TROLL-2 is knocked down by targeting the RPSAP52/NR 026825.2 isoform.

12. The method of treating a cancer in a subject of claim 11, wherein the expression of TROLL-2 is knocked down by administering to the subject the siRNA as set forth in SEQ ID NO: 13, SEQ ID NO: 14, and/or SEQ ID NO: 15.

13. The method of treating a cancer in a subject of claim 7, wherein the expression of TROLL-3 is knocked down by targeting the TRAF3IP2-AS1/NR_034108.1, TRAF3IP2-AS1/NR_034109.1, TRAF3IP2-AS1/NR_034110.1, and/or TRAF3IP2-AS1/NR_034111.1 isoform.

14. The method of treating a cancer in a subject of claim 13, wherein the expression of TROLL-3 is knocked down by administering to the subject the siRNA as set forth in SEQ ID NO: 16, SEQ ID NO: 17, and/or SEQ ID NO: 18.

15. The method of treating a cancer in a subject of claim 7, wherein the expression of TROLL-5 is knocked down by targeting the LINC00514/NR_033861.1 isoform.

16. The method of treating a cancer in a subject of claim 15, wherein the expression of TROLL-5 is knocked down by administering to the subject the siRNA as set forth in SEQ ID NO: 22, SEQ ID NO: 23, and/or SEQ ID NO: 24.

17. The method of treating a cancer in a subject of claim 7, wherein the expression of TROLL-7 is knocked down by targeting the MALAT1/NR_002819.4, MALAT1/NR_144567.1, and/or MALAT1/NR_144568.1 isoform.

18. The method of treating a cancer in a subject of claim 17, wherein the expression of TROLL-7 is knocked down by administering to the subject the siRNA as set forth in SEQ ID NO: 28, SEQ ID NO: 29, and/or SEQ ID NO: 30.

19. The method of treating a cancer in a subject of claim 7, wherein the expression of TROLL-8 is knocked down by targeting the ENST00000554568.1 isoform.

20. The method of treating a cancer in a subject of claim 19, wherein the expression of TROLL-8 is knocked down by administering to the subject the siRNA as set forth in SEQ ID NO: 31, SEQ ID NO: 32, and/or SEQ ID NO: 33.

21. The method of treating a cancer in a subject of claim 1 wherein the cancer is already established in the subject prior to the initiation of treatment.

22. The method of treating a cancer in a subject of claim 1 wherein the cancer is metastatic.

23. An siRNA comprising SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, or SEQ ID NO: 36.

24. A pharmaceutical composition comprising a therapeutically effective amount of one or more of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, or SEQ ID NO: 36, or any combination of 2, 3, 4, 5, 6, 7, 8, 9, 10, 11, 12, 13, 4, 15, 16, 17, 18, 19, 20, 21, 22, 23, 24, 25, 26, or all 27 siRNAs.

25. (canceled)
